(19) United States
(12) Patent Application Publication
(10) Pub. No.: US 2016/0295901 A1
(43) Pub. Date: Oct. 13, 2016

(54) PAENIBACILLUS STRAINS AND COMPOSITIONS THEREOF THAT INHIBIT MICROORGANISMS

(71) Applicant: DUPONT NUTRITION BIOSCIENCES APS, Copenhagen K (DK)

(72) Inventors: Matthew Hundt, Waukesha, WI (US); Elizabeth Galbraith, Wauwatosa, WI (US); Rebecca J. Watson, Waukesha, WI (US); Mari Ellen Davis, Waukesha, WI (US); Tina Mygind, Arhus (DE)

(21) Appl. No.: 15/102,074

(22) PCT Filed: Dec. 17, 2014
(86) PCT No.: PCT/IB2014/003172

§ 371 Date: Jun. 6, 2016

Related U.S. Application Data
(60) Provisional application No. 61/917,838, filed on Dec. 18, 2013.

(51) Int. Cl. A23L 3/3571; C12N 1/20; A01N 63/02; A01N 3/02; C12R 1/01; A01N 63/00

(52) U.S. Cl. CPC A23L 3/3571; C12R 1/01; A01N 63/00; A01N 63/02; A01N 3/02; C12N 1/20; A23V 2002/00

(57) ABSTRACT

The disclosure relates to Paenibacillus polymyxa strains, compositions of the strains, anti-contaminant compositions, and methods of using the strains and compositions to inhibit growth of microorganisms, including pathogens causing spoilage of products, such as human food, pet food, and flowers.

### CROSS REFERENCE TO RELATED APPLICATIONS

This application claims priority to and is a non-provisional application of U.S. Provisional Patent Application No. 61/917,838 filed Dec. 18, 2013, which is incorporated herein by reference in its entirety.

### BIBLIOGRAPHY

Complete bibliographic citations of the references referred to herein by the first author's last name in parentheses can be found in the Bibliography section, immediately preceding the claims.

### FIELD

The disclosure relates to Paenibacillus polymyxa strains, compositions of the strains, anti-contaminant compositions, and methods of using the strains and compositions to inhibit growth of microorganisms, including pathogens causing spoilage of products, such as human food, pet food, and flowers.

### BACKGROUND

Microorganisms, including bacteria and fungi, can cause disease in animals and plants. Microorganisms also cause food spoilage, food poisoning, and spoilage of cut flowers. Food spoilage occurs with fresh meat, fruit juices, infant formula, vegetables, and other foods. Food spoilage leads to waste and illness.

In addition, microorganisms can cause food poisoning in two different ways. Some microorganisms infect the intestines, causing inflammation and difficulty absorbing nutrients and water, leading to diarrhea. Other microorganisms produce toxins in foods that are poisonous to humans. When eaten, these toxins can lead to nausea, vomiting, kidney failure, and even death.

Microorganisms can also cause spoilage of cut flowers. Microorganisms grow in water that cut flowers are placed in. Growth of microorganisms clogs stems, thereby significantly shortening vase life.

In view of the foregoing, it would be desirable to provide a strain that is useful against one or more of these harmful microorganisms. It would also be useful to provide methods of making and using the strain.

### BRIEF SUMMARY

In one embodiment, the disclosure relates to isolated Paenibacillus polymyxa strains. In another embodiment, the disclosure relates to biologically pure cultures of Paenibacillus polymyxa strains, including but not limited to Paenibacillus polymyxa strain ABP-166.

In one embodiment, the Paenibacillus polymyxa strains have activity against microorganisms. In yet another embodiment, the Paenibacillus polymyxa strains inhibit the growth of microorganisms. In still another embodiment, the Paenibacillus polymyxa strains increase the longevity or lifespan of a product. In another embodiment, the Paenibacillus polymyxa strains can be used as a direct-fed microbial. In one embodiment, the Paenibacillus polymyxa strain is ABP-166.

Unless stated otherwise, compositions disclosed herein can comprise or consist of or consist essentially of various components.

In another embodiment, the disclosure relates to compositions comprising Paenibacillus polymyxa strains. In one embodiment, the compositions comprise Paenibacillus polymyxa strains and have activity against microorganisms. In yet another embodiment, the compositions comprise Paenibacillus polymyxa strains and can be used to inhibit the growth of microorganisms. In still another embodiment, the compositions comprise Paenibacillus polymyxa strains and can be used to increase the longevity or lifespan of a product. In another embodiment, the compositions comprise Paenibacillus polymyxa strains and can be used as a direct-fed microbial. In one embodiment, the Paenibacillus polymyxa strain in the compositions is ABP-166.

In still another embodiment, the disclosure relates to anti-contaminant compositions comprising Paenibacillus polymyxa strains. In one embodiment, the anti-contaminant compositions comprise Paenibacillus polymyxa strains and have activity against microorganisms. In yet another embodiment, the anti-contaminant compositions comprise Paenibacillus polymyxa strains and can be used to inhibit the growth of microorganisms. In still another embodiment, the anti-contaminant compositions comprise Paenibacillus polymyxa strains and can be used to increase the longevity or lifespan of a product. In another embodiment, the anti-contaminant compositions comprise Paenibacillus polymyxa strains and can be used as a direct-fed microbial. In one embodiment, the Paenibacillus polymyxa strain in the anti-contaminant compositions is ABP-166.

In yet another embodiment, the disclosure relates to a cell-free fermentation product from Paenibacillus polymyxa strains. In one embodiment, the cell-free fermentation product from Paenibacillus polymyxa strains has activity against microorganisms. In yet another embodiment, the cell-free fermentation product from Paenibacillus polymyxa strains inhibits the growth of microorganisms. In still another embodiment, the cell-free fermentation product from Paenibacillus polymyxa strains increases the longevity or lifespan of a product. In another embodiment, the cell-free fermentation product from Paenibacillus polymyxa strains can be used as a direct-fed microbial. In one embodiment, the cell-free fermentation product is from Paenibacillus polymyxa ABP-166.

In one embodiment, the disclosure relates to an isolated Paenibacillus polymyxa strain ABP-166, accession number B-50211. In another embodiment, the disclosure relates to a strain having all of the identifying characteristics of Paenibacillus polymyxa strain ABP-166. In still another embodiment, the disclosure relates to a derivative or variant of Paenibacillus polymyxa strain ABP-166.

In yet another embodiment, the disclosure relates to a cell-free fermentation product from Paenibacillus polymyxa strain ABP-166. In another embodiment, the disclosure relates to a cell-free fermentation product from a strain having all of the identifying characteristics of Paenibacillus polymyxa strain ABP-166. In another embodiment, the disclosure relates to a cell-free fermentation product from a derivative or variant of Paenibacillus polymyxa strain ABP-166.

In still another embodiment, the disclosure relates to a composition comprising Paenibacillus polymyxa strain ABP-166, or a strain having all of the identifying characteristics of Paenibacillus polymyxa strain ABP-166, or a derivative or variant of Paenibacillus polymyxa strain ABP-166 and a product. In one embodiment, the product is a foodstuff. In yet another embodiment, the foodstuff is selected from the group consisting of: human food, pet food, plant food, aquaculture food, animal feed, and feedstuff.

In one embodiment, the disclosure relates to a method of growing Paenibacillus polymyxa strain ABP-166, or a strain having all of the identifying characteristics of ABP-166, or a derivative or variant of ABP-166 comprising: (a) culturing Paenibacillus polymyxa strain ABP-166, or a strain having all of the identifying characteristics of ABP-166, or a derivative or variant of ABP-166 on, or in any one or more substrates; and (b) separating the strain from the substrate.

In yet another embodiment, the disclosure relates to a method of forming a direct-fed microbial comprising: (a) growing in a liquid nutrient broth, a culture including Paenibacillus polymyxa strain ABP-166, or a strain having all of the identifying characteristics of ABP-166, or a derivative or variant of ABP-166; and (b) separating the strain from the liquid nutrient broth.

In yet another embodiment, the disclosure relates to a method of producing an anti-contaminant composition comprising: (a) culturing Paenibacillus polymyxa strain ABP-166, or a strain having all of the identifying characteristics of ABP-166, or a derivative or variant of ABP-166, on, or in any one or more substrates to produce a fermentate; and (b) separating and/or inactivating at least some viable cells.

In one embodiment, the disclosure relates to a method of preventing and/or reducing microbial contaminant of a product comprising: contacting a constituent of the product, the product itself, and/or the packaging of the product with a strain disclosed herein.

In still another embodiment, the disclosure relates to a method of preventing and/or reducing microbial contaminant of a product comprising: contacting a constituent of the product, the product itself, and/or the packaging of the product with a composition disclosed herein.

In yet another embodiment, the disclosure relates to a method of preventing and/or reducing microbial contaminant of a product comprising: contacting a constituent of the product, the product itself, and/or the packaging of the product with an anti-contaminant composition disclosed herein.

In another embodiment, the disclosure relates to a method of inhibiting microorganisms comprising: administering a strain disclosed herein, and/or a composition disclosed herein, and/or the anti-contaminant composition disclosed herein, and/or the cell-free fermentation product disclosed herein to an environment potentially containing microorganisms.

In yet another embodiment, the disclosure relates to a method of inhibiting food spoilage comprising applying a strain disclosed herein, and/or a composition disclosed herein, and/or the anti-contaminant composition disclosed herein, and/or the cell-free fermentation product disclosed herein to a food grade film.

In yet another embodiment, the disclosure relates to a method of extending the longevity or lifespan of a product comprising: contacting a constituent of the product, the product itself, and/or the packaging of the product with a strain disclosed herein, and/or a composition disclosed herein, and/or the anti-contaminant composition disclosed herein, and/or the cell-free fermentation product disclosed herein.

An advantage of the strains, compositions, anti-contaminant compositions, cell-free fermentation products, and methods disclosed herein is that they exhibit a broad spectrum of inhibitory activity against microorganisms, including bacteria and fungi.

The strains, compositions, anti-contaminant compositions, cell-free fermentation products, and methods disclosed herein may be highly desirable in various industries, such as the food industry where consumers are demanding the use of more natural preservatives.

The strains, compositions, anti-contaminant compositions, cell-free fermentation products, and methods disclosed herein are cost-effective.

### BRIEF DESCRIPTION OF THE DRAWINGS

Exemplary embodiments of the invention are illustrated in the accompanying drawings.

FIG. 1 is a representative of photographs of agar plates highlighting the reduction in E. coli O157, dark arrows.

FIG. 2 is a representative photograph of an agar plate demonstrating cell-free fermentation product of ABP-166 inhibitory effect of Aliclycobacillus spp. isolate 1101. Sample was spotted in duplicate.

FIG. 3 is a representative bar graph depicting the percent reductions in plate counts of 9 Alicyclobacillus spp. isolates in the presence of 10% (v/v) cell-free fermentation product of ABP-166.

FIG. 4 is a representative bar graph depicting the results of a growth inhibition assay showing average plate count results for a mixture of 9 Aliclycobacillus isolates exposed to 10% (v/v) cell-free fermentation product of ABP-166.

FIG. 5 is a representative line graph depicting the results of a growth inhibition assay showing average of plate count results of vegetative cell growth for isolates 16-1 and 1101 exposed to 10% (v/v) cell-free fermentation product of ABP-166.

FIG. 6 is a representative line graph depicting the results of a growth inhibition assay showing average plate count results of spore out-growth for isolates 16-1 and 1101 exposed to 10% (v/v) cell-free fermentation product of ABP-166.

FIG. 7 is a representative bar graph depicting the inhibition of aerobic chicken spoilage isolates with cell-free fermentation product of ABP-166.

FIG. 8A-8F are photographs outlining the steps for an experiment with cell-free fermentation product of ABP-166 and chicken thighs. FIG. 8A is a photograph showing the chicken thighs set out flat side by side on flat trays. FIG. 8B is a photograph showing the inoculation of chicken thighs with S. putrefaciens. FIG. 8C is a photograph showing spray treatment of the chicken thighs. FIG. 8D is a photograph showing the vacuum chamber machine. FIG. 8E is a photograph showing chicken sealed in high barrier pouches. FIG. 8F is a photograph showing the vacuum sealed chicken stored at 4° C.

FIG. 9 is a representative chart depicting the odor panel data observed over 6 sampling time points for the trained “consumer” panelists as well as the day 14 data for the untrained “consumer” panelists.

FIG. 10A is a representative chart providing data from the overall reductions of H₂S-producing bacterial counts.

FIG. 10B is a representative line graph depicting data from the overall reductions of H₂S-producing bacterial counts.

FIGS. 11A and 11B are representative photographs of agar plates providing a visual representation of the reduction of H₂S-producing bacterial counts detected using H₂S media (black colonies). FIG. 11A is a representative photograph of an agar plate depicting the water treatment. FIG. 11B is a representative photograph of an agar plate depicting the cell-free fermentation product of ABP-166 treatment.

FIG. 12A is a representative chart and FIG. 12B is a representative line graph showing the reduction of total aerobic plate counts during progression of spoilage as compared to untreated samples (H₂O)

FIG. 13A is a representative chart and FIG. 13B is a representative line graph showing the reduction of total anaerobic plate counts during progression of spoilage as compared to untreated samples (H₂O).

FIG. 14 is a representative bar graph depicting the reduction of growth of numerous bacterial isolates as a result of the addition of cell-free fermentation product of ABP-166.

FIG. 15 is a dendogram that was constructed using the Pearson Correlation (with 80% cut-off relatedness) to determine the diversity among the S. putrefaciens isolates.

FIG. 16 is a representative bar graph depicting the activity of 10% cell-free fermentation product of ABP-166 against the 23 S, putrefaciens isolates. A cell-free fermentation product of ABP-166 had activity against all of the isolates tested.

FIG. 17 shows representative digital images of three human food pathogens, Salmonella (FIG. 17A and FIG. 17B), E. coli O157 (FIG. 17C and FIG. 17D), and L. monocytogenes (FIG. 17E and FIG. 17F) when treated with either a control film, top (A, C, and E), or one of the films that had been treated with cell-free fermentation product of ABP-166 (B, D, and F). The light colored arrows, seen in the panels, are pointing to the outline that is left from the film being in contact with the agars surface. The dark arrows indicate the irregular outline that can be seen where the application of cell-free fermentation product of ABP-166 inhibited the growth of the bacteria in the plate in contrast to the surrounding area where the bacteria were able to grow.

FIG. 18 is a representative bar graph depicting the results from the application of cell-free fermentation product of ABP-166 to food grade film. There was a reduction in the levels of L. monocytogenes at both 24 and 72 hours when compared to untreated control films.

FIG. 19 is a representative bar graph depicting the inhibition results for eleven isolates from flowers when tested against full-strength cell-free fermentation product of ABP-166 and two commercial products.

FIG. 20 is a schematic depicting the BAX® system diagnostic process.

FIG. 21 is a bar graph depicting direct plate counts of control kibble samples and samples treated with ABP 166. There was a reduction of Salmonella Newport (CFU/g of kibble) at 24 hours and 1 week by ABP166.

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range.

The headings provided herein are not limitations of the various aspects or embodiments of this disclosure, which can be had by reference to the specification as a whole.

Other definitions of terms may appear throughout the specification. Before the exemplary embodiments are described in more detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical, or other property, such as molecular weight, temperature, etc., is from 100 to 1,000, it is intended that all individual values, such as 100, 101, 102, etc., and sub ranges, such as 100 to 144, 155 to 170, 197 to 200, etc., are expressly enumerated. For ranges containing values that are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, various temperatures, and other parameter ranges recited in the methods.

As used herein, “administer” is meant to be the action of introducing the strain and/or supernatant to an environment in need of pathogen inhibition.

The terms “anti-contaminant composition” and “anti-contaminant agent,” as used herein, refers to any composition/agent which, in use, can counter (i.e. work in opposition to, hinder, oppose, reduce, prevent, or inhibit) the growth of a microorganism and/or which can, when used, counter (e.g. reduce or prevent or inhibit) the spoilage (preferably microbial spoilage) of a product. Thus, an “anti-contaminant” may be anti-pathogenic and/or anti-spoilage. In some aspects, an “anti-contaminant composition” may be a shelf-life extending composition.

In one embodiment, the anti-contaminant composition is a cell-free fermentation product. For example, the anti-contaminant composition of the present invention may simply be a fermentate, which has been modified to remove and/or to inactivate bacterial cells and/or spores to provide a cell-free fermentate.

The term “cell-free,” as used herein, means that the fermentation product (preferably the fermentate) is substantially free of viable bacterial cells, typically containing less than about 10⁵ viable bacterial cells/mL fermentation product, less than about 10⁴ viable bacterial cells/mL fermentation product, less than about 10³ viable bacterial cells/mL fermentation product, less than about 10² viable bacterial cells/mL fermentation product, or less than about 10 viable bacterial cells/mL fermentation product. In one embodiment, the fermentation product is substantially free of cells, typically containing less than about 10⁵ cells/mL fermentation product, less than about 10⁴ cells/mL fermentation product, less than about 10³ cells/mL fermentation product, less than about 10² cells/mL fermentation product, or less than about 10 cells/mL fermentation product.

The term “cell-free fermentation product,” as used herein, means a composition which results from culturing in a suitable media Paenibacillus polymyxa strain ABP-166 once some or all of the bacterial cells (including preferably spores) have been removed and/or inactivated; or a supernatant, a fraction, or a component thereof. In one embodiment, the cell-free fermentation product comprises at least one or more metabolites selected from the group consisting of a lipopeptide, a polyketide, a bacillibactin, a bacilysin, an anticapsin, a plantazolicin, a LCI, a homologue of a plantazolicin, a homologue of a LCI, polymyxin, fusaricidin, bacillorin, a polyketide, and one or more non-ribosomal peptide synthetases (NRPS).

In one embodiment, the compound(s) is/are a metabolite(s) of the bacteria being cultured (e.g. fermented).

As used herein, the term “contacting” includes both direct and indirect contact.

As used herein, the term “fermentate” refers to the mixture of constituents present during or following the culturing of Paenibacillus polymyxa strain ABP-166. The mixture of constituents may be present at any phase of culturing Paenibacillus polymyxa strain ABP-166, including but not limited to the initial growth phase, logarithmic growth phase or the lag growth phase.

Hence, the term “fermentate” can include one or more anti-contaminant compounds, such as: a lipopeptide (e.g. a surfactin), a bacilomycin (e.g. bacillomycin D), a fengycin (or combinations thereof), a polyketide (e.g. a difficidin, a macrolactin, a bacillaene, or combinations thereof), a bacillibactin, a bacilysin, an anticapsin, a plantazolicin, a LCI, a homologue of a plantazolicin, a homologue of a LCI, polymyxin, fusaricidin, bacillorin, a polyketide, and one or more non-ribosomal peptide synthetases (NRPS), as well as other components such as particulate matter, solids, substrates not utilized during culturing, debris, media, cell waste, etc. In one aspect, bacterial cells (preferably spores) are removed from the fermentate and/or inactivated to provide a cell-free fermentate.

In one embodiment, the fermentation product (preferably the fermentate) may be substantially free of viable bacterial cells, typically containing less than about 10² viable cells/mL fermentation product.

In another embodiment, the fermentation product (preferably the fermentate) may be substantially free of viable bacterial cells, typically containing less than about 10 viable cells/mL fermentation product.

In yet another embodiment, the fermentation product (preferably the fermentate) may be substantially free of viable bacterial cells, typically containing zero (or substantially) viable cells/mL fermentation product.

In some aspects, the term “cell-free” means that the fermentation product is substantially free of viable spores in addition to viable cells, typically containing less than about 10⁵ viable spores/mL fermentation product, less than about 10⁴ viable spores/mL fermentation product, less than about 10³ viable spores/mL fermentation product, less than about 10² viable spores/mL fermentation product, or less than about 10 viable spores/mL fermentation product. Preferably, the fermentation product is substantially free of spores, typically containing less than about 10⁵ spores/mL fermentation product, less than about 10⁴ spores/mL fermentation product, less than about 10³ spores/mL fermentation product, less than about 10² spores/mL fermentation product, or less than about 10 spores/mL fermentation product.

In one embodiment, the fermentation product (preferably the fermentate) may be substantially free of viable spores, typically containing less than about 10² viable spores/mL fermentation product.

In another embodiment, the fermentation product (preferably the fermentate) may be substantially free of viable spores, typically containing less than about 10 viable spores/mL fermentation product.

In yet another embodiment, the fermentation product (preferably the fermentate) may be substantially free of viable spores, typically containing zero (or substantially zero) viable spores/mL fermentation product.

In one aspect, the term “cell-free,” as used herein, means that the fermentation product (preferably the fermentate) is substantially free of viable bacterial cells and viable spores, typically containing less than about 10⁵ viable bacterial cells and viable spores/mL fermentation product, less than about 10⁴ viable bacterial cells and viable spores/mL fermentation product, less than about 10³ viable bacterial cells and viable spores/mL fermentation product, less than about 10² viable bacterial cells and viable spores/mL fermentation product, or less than about 10 viable bacterial cells and viable spores/mL fermentation product. Preferably, the fermentation product is substantially free of cells and/or spores, typically containing less than about 10⁵ cells and/or spores/mL fermentation product, less than about 10⁴ cells and/or spores/mL fermentation product, less than about 10³ cells and/or spores/mL fermentation product, less than about 10² cells and/or spores/mL fermentation product, or less than about 10 cells and/or spores/mL fermentation product.

In one embodiment, the fermentation product (preferably the fermentate) may be substantially free of viable bacterial cells and viable spores, typically containing less than about 10² viable cells and/or viable spores/mL fermentation product.

In another embodiment, the fermentation product (preferably the fermentate) may be substantially free of viable bacterial cells and viable spores, typically containing less than about 10 viable cells and/or viable spores/mL fermentation product.

In yet another embodiment, the fermentation product (preferably the fermentate) may be substantially free of viable bacterial cells and viable spores, typically containing zero (or substantially zero) viable cells and/or viable spores/mL fermentation product.

In some aspects, the fermentation product (preferably the fermentate) of the present invention may be treated (e.g. heat treated or irradiated) so that no cells, spores, or combinations thereof remain viable.

The term “contaminant,” as used herein, means any microorganism, such as a pathogenic microorganism, or spoilage microorganism. In one aspect, the term “contaminant” refers to a pathogenic microorganism and/or a spoilage microorganism.

As used herein, “effective amount” is meant to be a quantity of strain, composition, or anti-contaminant composition sufficient to inhibit growth of a microorganism or to impede the rate of growth. The amount of inhibition can be measured as described herein, or by other methods known in the art.

With regard to a strain, a composition or anti-contaminant composition fed to an animal or in a composition with a foodstuff for an animal, effective amount refers to a quantity of strain, composition or anti-contaminant composition to allow improvement in at least one of the following: the efficiency of animal production, carcass characteristics, growth performance of an animal, growth performance when feeding high levels of DDGS to an animal, nutrient digestibility, breakdown of complex dietary components, animal growth performance, poultry growth performance, pig growth performance, feed efficiency, average daily gain, average daily feed intake, body weight gain:feed or feed:gain intake, and morality.

As used herein, “food grade film” refers to a substrate for use as a food wrap.

As used herein, “food product” refers to a substance that can be used or prepared for use as food. The food product can be a substance ingested by humans, non-humans, mammals, reptiles, cattle, cats, dogs, goats, swine, pigs, monkeys, apes, gorillas, bulls, cows, bears, horses, sheep, poultry, mice, rats, fish, shrimp, dolphins, whales, and sharks. The food product can be pet food, animal food, or food for human consumption.

In one embodiment, the food product can be a product consumed by plants.

In another embodiment, the food product can be a feedstuff for administration to an animal. In yet another embodiment, the food product can be a feed material.

In another embodiment, the food product can be pet food. The pet food can be consumed by any pet, including but not limited to dogs, cats, rabbits, mice, gerbils, snakes, fish, birds, lamas, alpaca, goats, sheep, chickens, and marine animals. Food product includes, but not limited to: fresh meats, processed meats, poultry, fish and other seafood, dairy products, bakery products, eggs in the shell, fresh fruits, fresh vegetables, food that is gleaned, food that is packaged, refrigerated, or frozen, and food that is canned or jarred.

As used herein, “food spoilage” includes any alteration in the condition of food which makes it less palatable, including changes in taste, smell, texture, appearance, change in pH, change in moisture content, change in temperature, change in consistency, change in color, or a change in structure. Spoiled food may or may not be toxic.

As used herein, “food poisoning” refers to an illness caused by the consumption of food or water contaminated with bacteria, viruses, molds, or with other parasites and/or their toxins. The symptoms, varying in degree and combination, include: abdominal pain, vomiting, diarrhea, and headache; more serious cases can result in life-threatening neurologic, hepatic, and renal syndromes leading to permanent disability or death. The term “food poisoning” may be used interchangeably with “food-borne disease” and “food-borne illness.

The term “inhibit,” as used herein, means to destroy, prevent, control, decrease, slow or otherwise interfere with the growth or survival of a contaminant microorganism when compared to the growth or survival of the contaminant microorganism in an untreated control. In one aspect, to “inhibit” is to destroy, prevent, control, decrease, slow, or otherwise interfere with the growth or survival of a contaminant microorganism by at least about 3% to at least about 100%, or any value in between, for example, at least about 10%, 15%, 20%, 25%, 30%, 35%, 40%, 45%, 50%, 55%, 60%, 65%, 70%, 75%, 80%, 85%, 90%, 95%, 99%, or 100% when compared to the growth or survival of the contaminant microorganism in an untreated control.

In another aspect, to “inhibit” is to destroy, prevent, control, decrease, slow, or otherwise interfere with the growth or survival of a contaminant microorganism by at least about 1-fold or more, for example, about 1.5-fold to about 100-fold, or any value in between by at least about 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95-fold when compared to the growth or survival of the contaminant microorganism in an untreated control.

The term “pathogenic microorganism,” as used herein, refers to a microorganism which is capable of causing disease in a human, an animal, or a plant. The “pathogenic microorganism” may be present at any point in the lifetime of a product, for example, originating from one or more of the following: the environment from which the product was obtained and/or the microbiological quality of the product in its raw or unprocessed state (e.g. native to the product), and/or any handling, processing steps, the effectiveness/ineffectiveness of packaging, and/or storage conditions of the product.

In one aspect, the term “preventing,” as used herein, means the microbial contamination of a product which comprises a strain, a composition, or an anti-contaminant composition or a product to which strain, a composition, or an anti-contaminant composition is applied has an extended shelf-life and/or increased time frame before a specified amount of contaminant is present. In one embodiment, shelf-life and/or time frame is extended and/or increased when compared to a control product which does not have a strain, a composition, or an anti-contaminant composition applied.

For example, when the contaminant is a pathogenic microorganism (e.g. a pathogen bacterium), the “specified amount of contaminant” may be the level at which a product is deemed not to be safe for use by, for example, the FDA. In some instances, depending on the pathogenic microorganism, the specified amount of contaminant may be zero. This may be the case when the pathogenic microorganism is Listeria spp., for example. In other instances, the specified amount of contaminant may be less than about 100 CFU/g or ml, or less than about 10 CFU/g or ml, such as when the pathogenic bacteria is e.g., E. coli spp.

When the contaminant is a non-pathogenic spoilage bacteria, the “specified amount of contaminant” may be the level at which the organoleptic conditions are no longer acceptable or the level at which the consumer visualizes the spoilage of the product. The specified amount may be dependent on the microorganism. However, in some instances, it may be the presence of e.g., 10³ or 10⁴ CFU/g or CFU/ml.

The term “reducing,” as used herein, in relation to microbial contaminant, means that the level of microbial growth and/or speed at which a product spoils is reduced when compared to a control product to which no strain, composition, or anti-contaminant has been applied. In one aspect, the terms “reduce” and “reducing” may be used interchangeably with the terms “inhibit” and “inhibiting.”

The term “spoilage microorganism,” as used herein, refers to a microorganism which can cause detrimental changes in appearance, flavor, odor, and other qualities of the product, preferably which results from microbial growth. The “spoilage microorganism” may be present at any point in the lifetime of a product, for example, originating from one or more of the following: the environment from which the product was obtained and/or the microbiological quality of the product in its raw or unprocessed state (e.g. native to the product), and/or any handling, processing steps, the effectiveness/ineffectiveness of packaging, and/or storage conditions of the product.

The term “viable,” as used herein, means a microbial cell or spore that is metabolically active or able to differentiate. Thus, with regard to spores, they are “viable” when they are dormant and capable of germinating.

The inventors have found that Paenibacillus polymyxa strains have activity against microorganisms, including bacteria and fungi. The inventors have also found that compositions comprising Paenibacillus polymyxa strains are active against microorganisms, including bacteria and fungi. The inventors have also found that anti-contaminant compositions that comprise a fermentation product of Paenibacillus polymyxa strains are active against microorganisms, including harmful bacteria and fungi. In one embodiment, the Paenibacillus polymyxa strain is Paenibacillus polymyxa strain ABP-166.

Paenibacillus polymyxa strain ABP-166 was deposited by Danisco USA, Inc. of Waukesha, Wis. at the Agricultural Research Service Culture Collection (NRRL), 1815 North University Street, Peoria, III., 61604 on Dec. 18, 2008 under accession number B-50211. The deposit was made under the provisions of the Budapest Treaty on the International Recognition of the Deposit of Microorganisms for the Purposes of Patent Procedure.

Danisco USA Inc. of W227 N752 Westmound Dr. Waukesha, Wis. 53186, USA authorize DuPont Nutrition Biosciences ApS (formerly Danisco A/S) of Langebrogade 1, PO Box 17, DK-1001, Copenhagen K, Denmark to refer to these deposited biological materials in this patent application and have given unreserved and irrevocable consent to the deposited material being made available to the public.

Any Paenibacillus derivative or variant and their compositions, and anti-contaminant compositions are also included and are useful in the methods described and claimed herein. Strains having all of the identifying characteristics of Paenibacillus polymyxa strain ABP-166 and compositions and anti-contaminant compositions of these strains are also included and are useful in the methods described and claimed herein.

In some embodiments, Paenibacillus polymyxa strain ABP-166, a composition comprising Paenibacillus polymyxa strain ABP-166 or an anti-contaminant composition comprising Paenibacillus polymyxa strain ABP-166 is used for inhibiting microorganisms, including bacteria and fungi. In another embodiment, an anti-contaminant composition comprising a cell-free fermentation product of Paenibacillus polymyxa strain ABP-166 is used for inhibiting microorganisms, including bacteria and fungi.

For example, Paenibacillus polymyxa strain ABP-166, a composition comprising Paenibacillus polymyxa strain ABP-166, an anti-contaminant composition comprising Paenibacillus polymyxa strain ABP-166, or combinations thereof can be used for inhibiting microorganisms, pathogens, and bacteria that cause food spoilage, food poisoning or food-borne illnesses, and/or spoilage of cut flowers.

In another embodiment, Paenibacillus polymyxa strain ABP-166, a composition comprising Paenibacillus polymyxa strain ABP-166, an anti-contaminant composition comprising Paenibacillus polymyxa strain ABP-166, or combinations thereof can be used for inhibiting plant pathogens, urinary tract pathogens, livestock pathogens, and aquaculture pathogens.

In another embodiment, Paenibacillus polymyxa strain ABP-166, a composition comprising Paenibacillus polymyxa strain ABP-166, an anti-contaminant composition comprising Paenibacillus polymyxa strain ABP-166, or combinations thereof can be used to extend the longevity or lifespan of a product. In one embodiment, the product is a foodstuff. In another embodiment, the foodstuff is human food, pet food, or feedstuff. In yet another embodiment, the product is flowers.

I. Paenibacillus polymyxa Strain ABP-166

Strain ABP-166 was initially isolated from a forage sample taken from an active dairy farm in northern Wisconsin. This strain was subsequently found in samples taken from the total mixed ration, corn and haylage, rumen contents, and fecal matter. The strain was originally isolated because it possessed a high degree of inhibition against E. coli O157.

In certain embodiments, any derivative or variant of Paenibacillus polymyxa strain ABP-166, compositions thereof, and anti-contaminant compositions of these strains are also included and are useful in the methods described and claimed herein.

As used herein, a “variant” has at least 80% identity of genetic sequences with the disclosed strains using random amplified polymorphic DNA polymerase chain reaction (RAPD-PCR) analysis. The degree of identity of genetic sequences can vary. In some embodiments, the variant has at least 85%, 90%, 95%, 96%, 97%, 98%, or 99% identity of genetic sequences with the disclosed strains using RAPD-PCR analysis. RAPD analysis can be performed using Ready-to-Go™ RAPD Analysis Beads (Amersham Biosciences, Sweden), which are designed as pre-mixed, pre-dispensed reactions for performing RAPD analysis. The Ready-to-go RAPD™ Analysis Beads primers and instruction manual are incorporated herein by reference.

The strain can be grown in various media, including TSB, IM1, IM2, and a milk-based media. Paenibacillus polymyxa strain ABP-166 is produced by fermentation. Activity can vary depending upon the media used.

Safety of strain ABP-166 was tested using a PCR based assay. Strain ABP-166 tested negative for four toxin genes that can be associated with Bacillus spp. These toxin genes were diarrheal toxin (nheB), emetic toxin (bceT), three-component hemolysin (hbl), and enterotoxin FM (entFM).

In at least some embodiments, Paenibacillus polymyxa strain ABP-166, compositions comprising Paenibacillus polymyxa strain ABP-166, and anti-contaminant compositions comprising Paenibacillus polymyxa strain ABP-166 may be used in combination.

A. Culturing Paenibacillus polymyxa Strains

The medium used to cultivate the cells may be any conventional medium suitable for growing Paenibacillus polymyxa strains. In one embodiment, Paenibacillus polymyxa strain ABP-166 may be grown under any conditions suitable for obtaining a fermentation product comprising a compound of interest.

The culturing can take place with, on, or in the presence of one or more substrates (e.g. a fermentable substrate). A fermentable substrate is a material that contains an organic compound such as a carbohydrate that can be transformed (i.e., converted into another compound) by the enzymatic action of a bacterium as disclosed herein.

Examples of substrates include, but are not limited to: non-fat dry milk, vegetables (e.g., corn potatoes, cabbage), starch, grains (e.g., rice, wheat, barley, hops), fruit (e.g., grapes, apples, oranges), sugar, sugarcane, meat (e.g., beef, poultry, pork, sausage), heart infusion, cultured dextrose, combinations thereof, and the like and suitable media containing proteins, carbohydrates, and minerals necessary for optimal growth. A non-limiting exemplary medium is TSB or CASO broth.

In one embodiment, the substrate may include one or more of: starch, soy, yeast extracts and salts. In yet another embodiment, the growth medium may be CASO broth. In still another embodiment, the growth medium may be TSB broth.

The culturing of a Paenibacillus polymyxa strain ABP-166 can take place for any suitable time. In one embodiment, culturing ABP-166 take place for a period of time conducive to produce a compound of interest. For example, the culturing can take place from about 1 to about 72 hours (h), from about 5 to about 60 h, from about 10 to about 54 h, or from 24 to 48 h. In one aspect the culturing can suitably take place for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 42, 48, 54, or 60 h, where any of the stated values can form an upper or lower endpoint when appropriate. In another aspect, the time for culturing can be greater than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 h. In yet another aspect, the time for culturing can be less than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 h. In still another aspect, suitably, the culturing occurs for approximately 24 to 48 hours.

In yet another embodiment, the culturing occurs for approximately 20 to 30 hours. In another embodiment, the culturing can be carried out until nutrient depletion (preferably complete nutrient) occurs.

In one embodiment, the culturing is for a time effective to reach the stationary phase of growth of the bacteria. The temperature during the culturing can be from about 20 to about 55° C., from about 25 to about 40° C., or from about 30 to about 35° C. In one aspect, the temperature during the culturing can be from about 20 to about 30° C., from about 30 to about 40° C., or from about 40 to about 50° C. In another aspect, the culturing can take place at a temperature of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C., where any of the stated values can form an upper or lower endpoint when appropriate. In still another aspect, the culturing can take place at a temperature greater than or equal to about, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C. In yet another aspect, the culturing can take place at a temperature less than or equal to about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C.

In one embodiment, the culturing can occur from about 30 to about 35° C. In a further aspect, the culturing can occur at about 32° C.

In yet another embodiment, the culturing preferably may take place under aeration. Suitably, the level of the aeration is controlled. Aeration levels may be expressed as dissolved oxygen tension (DOT), wherein DOT is a percentage of oxygen saturation in the culture, (e.g. 100% DOT means a culture is fully saturated with oxygen). DOT may be measured, as taught in Suresh et al “Techniques for oxygen transfer measurement in bioreactors: a review” J Chem Technol Biotechnol 2009; 84: 1091-1103 (and references therein), which is incorporated herein by reference, or as taught in Bailey J, Bailey J, Ollis D, “Biochemical Engineering Fundamentals”, 2^{nd} edition, McGraw-Hill, ISBN 0070032122 (and references therein) which is incorporated herein by reference.

In another embodiment, culturing occurs under conditions where oxygen content is not limiting. In one embodiment, the level of the aeration is such that the oxygen content in the culture is more than about 20% DOT, more than about 30% DOT, more than about 40% DOT, more than about 50% DOT, more than about 60% DOT, more than about 70% DOT, more than about 80% DOT or more than about 90% DOT. In some aspects, the level of aeration is such that the level of the aeration in the culture is about 100% DOT.

In one embodiment, the level of aeration is such that the oxygen content in the culture may be between about 25% and 50% DOT.

The aeration may be provided by any suitable method.

In some embodiments, the aeration may be provided by any means that mixes air with the culture. Thus, the aeration may be provided by agitation (e.g. shaking, oscillation, stirring etc.) or by passing air (e.g. oxygen) through the culture media, for example, or combination thereof.

The rate of aeration expressed as vvm (the volume of gas per liquid volume per minute) may be measured, as taught in Bailey J, Bailey J, Ollis D, “Biochemical Engineering Fundamentals”, 2^{nd} edition, McGraw-Hill, ISBN 0070032122 (and references therein), which is incorporated herein by reference, for example.

In one embodiment, the aeration rate may be in the range of about 0.1 to about 6 vvm. Where the aeration is provided by agitation (e.g. in a stirred fermentor), then the aeration rate may be in the range of about 0.1 to about 3 vvm. Where the aeration is provided by passing air through the culture media (e.g. in an airlift fermentor), then the aeration rate may be in the range of about 3 to about 6 vvm.

In still another embodiment, a culture container which is designed or shaped to support or provide aeration may be used. Suitably, the culture container may comprise one or more baffles. The aim of the baffles may be to encourage exposure of the media to oxygen (e.g. air). For example, a culture container with baffles may be used in combination with shaking or oscillation of the culture container. By way of example, only the culture container may be the container described in U.S. Pat. No. 7,381,559 (the subject matter of which is incorporated herein by reference).

Suitably, the culture medium may be agitated. This may be affected by any conventional means. Without wishing to be bound by theory, agitation of a culture medium may have a number of beneficial effects when compared to a non-agitated culture medium, including but not limited to: increased growth, and/or decreased cell clumping, and/or increased nutrient (e.g. carbohydrate) mixing, and/or better nutrient distribution, and/or increased protein production, and/or increased primary metabolite production, and/or increased secondary metabolite production, etc. In one aspect, the beneficial effects derived from agitating a culture medium may result from the creation of turbulence within the culture medium (e.g. by stirring). In one embodiment the agitation may be stirring. In another embodiment, the agitation may be shaking or oscillation.

In one aspect, the culture media is agitated by oscillation (e.g. by rotatory shaking). Suitably, the speed of rotation may be at about 50 to about 250 rpm, about 60 rpm to about 240 rpm, about 70 rpm to about 230 rpm, about 80 rpm to about 220 rpm, about 80 rpm to about 210 rpm, or about 90 rpm to about 200 rpm.

Suitably, the speed of rotation may be at about 100 rpm to about 150 rpm. Suitably, the speed of rotation may be at about 130 rpm.

In one embodiment, the culture medium is agitated in order to increase the level of aeration in the culture media and/or increase nutrient mixing in the culture media. It has been found that aeration and/or agitation of the culture mixture may result in significant improvements in the fermentate produced. Without wishing to be bound by theory, this improvement may be caused by ensuring the cell density or cell mass in the culture container is such that the protein yield and/or primary metabolite production by the bacteria is enhance in the fermentate.

In one aspect, the culture media may be agitated by stirring. The speed of stirring may be suitably greater than about 50 rpm, for example, between about 50 rpm to about 1200 rpm. The rate at which the culture media may be stirred, may be dependent upon the container in which it is held for culturing purposes. If the container comprising the culture media is a small fermentor (e.g. less than 500 L, such as about 100 to about 500 L or even less than 20 L), then the speed of stirring may be at least about 100 rpm to about 1200 rpm, for example. In some aspects, the speed of stirring may be greater than about 1200 rpm. If the container comprising the culture media is an industrial scale fermentor (e.g. great than 500 L, such as about 500 to about 20,000 L), then the speed of stirring may be at least about 50 rpm to about 150 rpm or may be greater than about 150 rpm, for example.

In another aspect, agitation of a culture media during culturing may be represented as power input by agitation for example. Power input by agitation is a representation of the amount of energy provided per liter of liquid volume. The power input by agitation can be calculated by first determining the power in Newton using the following formula:

P₀=N₀ρN³D³

where: N₀ is a dimensionless number (Newton number); ρ is the density of the liquid (kg/m³); N (s⁻¹) is the rotational frequency; and D is the impeller diameter (m). P₀ is the power drawn by an agitator when the culture is not aerated. Calculation of power input by agitation in the presence of aeration is taught in Olmos et al. “Effects of bioreactor hydrodynamics on the physiology of Streptomyces”, Bioprocess Biosyst Eng, 2012 Aug. 25 and references therein, which is incorporated herein by reference.

In one aspect, during culturing, the power input by agitation per volume may be at least about 0.25 kW/m³. In another embodiment, power input by agitation per volume may be in the range of about 0.25 kW/m³ to about 6 kW/m³. In another aspect, the power input by agitation per volume may be in the range of about 0.25 kW/m³ to about 3 kW/m³.

In another aspect, the culture volume to the container volume may be less than about 1:1 v/v, e.g. 1:2, 1:3, etc. In some aspects, the ratio of the culture volume to the container volume may be less than about 1:1 v/v, 1:2 v/v, 1:3 v/v, 1:4 v/v, 1:5 v/v, 1:6 v/v, 1:7 v/v, 1:8 v/v, 1:9 v/v, or 1:10 v/v.

In some aspects, the ratio of the culture volume to the container volume may be in the range of about 1:1 v/v to about 1:10 v/v, suitably, in the range of 1:3 v/v to about 1:7 v/v.

In some aspects, the ratio of the culture volume to the container volume may be about 1:1 v/v, 1:2 v/v, 1:3 v/v, 1:4 v/v, 1:5 v/v, 1:6 v/v, 1:7 v/v, 1:8 v/v, 1:9 v/v or 1:10 v/v.

Suitably, the ratio of the culture volume to the container volume may be about 1:5 v/v.

In one embodiment, the volume of culture may be less than about 100%, less than about 90%, less than about 80%, less than about 70%, less than about 60%, less than about 50%, less than about 40%, or less than about 30% that of the container volume, for example.

In another embodiment, the volume of the culture may be in the range of about 60% to about 90% that of the container volume, for example.

In still another embodiment, the volume of the culture may be in the range of about 70% to about 85% that of the container volume, for example.

The pH during the culturing can be at a pH from about 5 to about 9, from about 5 to about 6, from about 6 to about 7, from about 7 to about 8, or from about 8 to about 9. In another aspect, the culturing can take place at a pH of about 5, 6, 7, 8, 9, where any of the stated values can form an upper or lower endpoint when appropriate. In one aspect, the pH is at a pH between about 7 and about 8, from about 7 to about 7.5, or from about 7.1 to about 7.3 during the culturing. In one aspect, the culturing is at about pH 7.3.

Alternatively, or in addition, the pH may be adjusted after culturing to a pH from about 6 to about 10, from about 8 to about 10, or from about 9 to 10. Suitably, the pH may be adjusted from about pH 8 to about pH 9. Suitably, the pH may be adjusted to about pH 9.

In one embodiment, an alkali may be used to increase the pH. Suitably, potassium hydroxide (KOH) may be used.

In yet another embodiment, the pH is adjusted after separation of the bacterial cells and culture media (e.g. by centrifugation). Suitably, it is the pH of the supernatant which is adjusted.

In one embodiment, the culturing step comprises one or more adjustments of the culture conditions (such as an adjustment of pH, temperature, and/or substrate) during the culturing phase. Without wishing to be bound by theory, adjusting the culture conditions (e.g. pH, temperature, and/or substrate) during the culturing may increase the number of compounds of interest produced during the culturing process. For example, the initial culture conditions may be conducive to produce one compound of interest and the adjustment of the culture conditions may provide favorable conditions to produce a further compound of interest.

Thus, for example, during the culturing process, an initial pH of about pH 5 may produce one compound of interest. Subsequent adjustment of the pH to pH 7 during the same culturing process may result in the production of a further compound of interest.

Batch and continuous culturing are known to a person of ordinary skill in the art. The fermentation product of the present invention or a portion thereof comprising compound(s) of interest may be prepared using batch or continuous culturing. Suitably, the fermentation product, or a portion thereof, may be harvested during or at the end of the culturing process.

In still another embodiment, the fermentation product of the present invention is harvested during or at the end of the exponential phase. In one aspect, the fermentation product of the present invention is harvested at or during the stationary phase.

In another embodiment, the fermentation product may be produced in a vat under commercial conditions.

In a non-limiting example, Paenibacillus polymyxa ABP-166 is cultured to between about a 1×10⁹ CFU/ml to about a 1×10¹⁰ CFU/ml. This Paenibacillus strain can be grown in Tryptic Soy Broth (TSB), Milk Based Media, or Optimized Industrial Media de Man, Rogosa, and Sharpe (MRS) broth at 32° C. for 24 hours. The bacteria can be harvested by centrifugation and the supernatant removed.

B. Separating One or More Cells and/or Spores from the Fermentation Product

In one embodiment, one or more cells and/or one or more spores may be separated from the fermentation product (e.g., fermentate). Such separation may be achieved by any means known in the art, including by centrifuging and/or filtering. For example, the fermentation product can be filtered (one or several times in a multistep process) to remove such components as particulate matter, cells, spores, and the like. Alternatively, or in addition, one or more cells and/or one of more spores may be separated from the fermentation product (e.g. fermentate) by centrifugation, thus producing a supernatant. Depending on the speed and duration of the centrifugation, the supernatant can be cell free (i.e., a cell-free supernatant) or the supernatant can contain cells, which can be filtered or further centrifuged to provide a cell-free supernatant.

In one embodiment, the method of separation is or includes centrifugation. Centrifugation is well known in the art. Centrifugation may be carried out at, for example, about 5,000 rpm, 10,000 rpm, 15,000 rpm, 20,000 rpm, 25,000 rpm, or 30,000 rpm. In one aspect, the speed of the centrifugation can be at least about 5,000 rpm.

In one embodiment, centrifugation may be carried out between about 5,000 rpm to between about 15,000 rpm.

In another embodiment, centrifugation may be carried out at about 5,000×g to about 15,000×g, or at about 10,000×g to about 20,000×g.

In still another embodiment, centrifugation may be carried out at about 9,000×g to about 12,000×g. Suitably, at about 11,000×g to about 14,000×g.

The time of centrifugation can be from about 5 minutes to 1 h, from about 10 minutes to about 45 minutes, or about 30 minutes. In one aspect, the time of the centrifugation is at least about 10 minutes, or at least about 15 minutes.

In one embodiment, the time of centrifugation can be from about 20 to about 40 minutes. In another embodiment, the time of centrifugation can be from about 5 to about 15 minutes.

In yet another embodiment, centrifugation is performed two or more times, using either the same or different centrifugation conditions.

In still another embodiment, one or more cells and/or one or more spores can be separated from the fermentate or supernatant (e.g., after centrifugation) by filtration. Various filters can be used to filter the fermentate or a supernatant containing cells and/or spores. For example, a micro filter with a pore size of from about 0.01 to about 1 μm, from about 0.05 to about 0.5 μm, or from about 0.1 to about 0.2 μm.

In another embodiment, the filter can have a pore size of about 0.01, 0.02, 0.03, 0.04, 0.05, 0.06, 0.07, 0.08, 0.09, 0.1, 0.2, 0.3, 0.4, 0.5, 0.6, 0.8, 0.9, or 1 urn, where any of the stated values can form an upper or lower endpoint when appropriate. In yet another embodiment, the filter can have a pore size of greater than or equal to about 0.01, 0.02, 0.03, 0.04, 0.05, 0.06, 0.07, 0.08, 0.09, 0.1, 0.2, 0.3, 0.4, 0.5, 0.6, 0.8, 0.9, or 1 μm. In still another embodiment, the filter can have a pore size of less than or equal to about 0.01, 0.02, 0.03, 0.04, 0.05, 0.06, 0.07, 0.08, 0.09, 0.1, 0.2, 0.3, 0.4, 0.5, 0.6, 0.8, 0.9, or 1 μm. In a further embodiment, the filter can have a pore size of about 0.2 μm, such as is available from Millipore (Billerica, Mass.). The fermentate can, in one aspect, be filtered with a sterilizing filter.

In another embodiment, the fermentate or supernatant may be filtered, e.g. with a sterilizing filter. In one embodiment, the filter (e.g. the sterilizing filter) may have a pore size of about 0.1 μm to about 0.3 μm. In another embodiment, the filter may have a pore size of about 0.2 μm. The resultant product may be considered a cell-free fermentation product in accordance with the present invention.

In still another embodiment, the anti-contaminant composition or cell-free fermentate in accordance with the present disclosure may be freeze-dried. Freeze-drying can be carried out by any suitable freeze-drying procedure. Freeze-drying may be carried out for between about 1 hour to about 10 days, between about 1 day to about 8 days, suitably, between about 1 day to about 5 days.

C. Inactivating One or More Cells and/or Spores

Methods for the inactivation of viable cells are well known in the art and include heat-treatment and irradiation. Any known means for inactivating viable cells may be employed, provided that they would not also inactivate the compound or compounds of interest in accordance with the present invention.

In one embodiment, inactivation of viable cells can be achieved using heat-treatment. Suitable methods of heat treatment are known in the art and include the following conditions:
a. LTLT pasteurization (e.g. 63° C. for 30 minutes);
b. HTST pasteurization (e.g. 72-75° C. for 15-20 seconds or >80° C. for 1-5 seconds);
c. Ultra pasteurization (e.g. 125-138° C. for 2-4 seconds);
d. UHT flow sterilization (e.g. 135-140° C. for 1-2 seconds); and
e. Sterilization in a container (e.g. 115-120° C. for 20-30 minutes).

Such methods of heat treatment may be combined with vacuum or reduced pressure.

In one embodiment, inactivation of spores may be achieved using heat treatment such as using the UHT flow sterilization or sterilization in a container, conditions provided above.

Separation and/or inactivation of spores may be by filter sterilization of the culture supernatant after centrifugal ion and discharge of the pellet containing the cells and spores.

Alternatively or additionally, double pasteurization could be used. For example, this could comprise a first pasteurization step (e.g. using the UHT flow sterilization or sterilization in a container, conditions provided above), incubation of a product at a temperature and for a time which induces spore germination, and a second pasteurization to heat inactivate the new vegetative forms of cells.

### II. DFMs and Methods of Preparing a DFM

In one embodiment, the disclosure relates to a composition comprising Paenibacillus polymyxa strains. In another embodiment, the disclosure relates to a composition comprising Paenibacillus polymyxa strain ABP-166, a variant thereof or a derivative thereof. The composition can be fed to an animal as a direct-fed microbial (DFM). One or more carrier(s) or other ingredients can be added to the DFM. The DFM may be presented in various physical forms, for example, as a top dress, as a water soluble concentrate for use as a liquid drench or to be added to a milk replacer, gelatin capsule, or gels. In one embodiment of the top dress form, freeze-dried bacteria fermentation product is added to a carrier, such as whey, maltodextrin, sucrose, dextrose, limestone (calcium carbonate), rice hulls, yeast culture, dried starch, and/or sodium silico aluminate. In one embodiment of the water soluble concentrate for a liquid drench or milk replacer supplement, freeze-dried bacteria fermentation product is added to a water soluble carrier, such as whey, maltodextrin, sucrose, dextrose, dried starch, sodium silico aluminate, and a liquid is added to form the drench or the supplement is added to milk or a milk replacer. In one embodiment of the gelatin capsule form, freeze-dried bacteria fermentation product is added to a carrier, such as whey, maltodextrin, sugar, limestone (calcium carbonate), rice hulls, yeast culture dried starch, and/or sodium silico aluminate. In one embodiment, the bacteria and carrier are enclosed in a degradable gelatin capsule. In one embodiment of the gels form, freeze-dried bacteria fermentation product is added to a carrier, such as vegetable oil, sucrose, silicon dioxide, polysorbate 80, propylene glycol, butylated hydroxyanisole, citric acid, ethoxyquin, and/or artificial coloring to form the gel.

The strain(s) may optionally be admixed with a dry formulation of additives, including but not limited to: growth substrates, enzymes, sugars, carbohydrates, extracts and growth promoting micro-ingredients. The sugars could include the following: lactose; maltose; dextrose; malto-dextrin; glucose; fructose; man nose; tagatose; sorbose; raffinose; and galactose. The sugars range from 50-95%, either individually or in combination. The extracts could include yeast or dried yeast fermentation solubles ranging from 5-50%. The growth substrates could include: trypticase, ranging from 5-25%; sodium lactate, ranging from 5-30%; and, Tween 80, ranging from 1-5%. The carbohydrates could include: mannitol, sorbitol, adonitol, and arabitol. The carbohydrates range from 5-50% individually or in combination. The micro-ingredients could include the following: calcium carbonate, ranging from 0.5-5.0%; calcium chloride, ranging from 0.5-5.0%; dipotassium phosphate, ranging from 0.5-5.0%; calcium phosphate, ranging from 0.5-5.0%; manganese proteinate, ranging from 0.25-1.00%; and, manganese, ranging from 0.25-1.0%.

To prepare DFMs described herein, the culture(s) and carrier(s) (where used) can be added to a ribbon or paddle mixer and mixed for about 15 minutes, although the timing can be increased or decreased. The components are blended such that a uniform mixture of the cultures and carriers result. The final product is preferably a dry, flowable powder. The strain(s) can then be added to animal feed or a feed premix, added to an animal's water, or administered in other ways known in the art. A feed for an animal can be supplemented with one or more strain(s) described herein or with a composition described herein.

In one embodiment, the disclosure relates to a method of forming a direct fed microbial comprising growing in a liquid nutrient broth a culture including a strain of Paenibacillus polymyxa and separating the strain from the liquid nutrient broth. In one embodiment, Paenibacillus polymyxa is strain ABP-166, a variant thereof or a derivative thereof. In another embodiment, the bacterial strain is grown to a level of about 1×10⁵ CFU/ml to about 5×10⁹ CFU/ml.

The DFM provided herein can be administered, for example, as the strain-containing culture solution, the strain-producing anti-contaminant composition, or the bacterial product of a culture solution.

Administration of a DFM provided herein to an animal can increase the performance of the animal. In one embodiment, administration of a DFM provided herein to an animal can increase the average daily feed intake (ADFI), average daily gain (ADG), or feed efficiency (gain:feed; G:F or feed:gain; F:G) (collectively, “performance metrics”). One or more than one of these performance metrics may be improved.

The DFM may be administered to the animal in one of many ways. For example, the strain(s) can be administered in a solid form as a veterinary pharmaceutical, may be distributed in an excipient, preferably water, and directly fed to the animal, may be physically mixed with feed material in a dry form, or the strain(s) may be formed into a solution and thereafter sprayed onto feed material. The method of administration of the strain(s) to the animal is considered to be within the skill of the artisan.

A. Methods of Administering to an Animal

In one embodiment, the strains can be administered in an effective amount to animals. In at least some embodiments, the disclosure relates to a method comprising administering to an animal an effective amount of a Paenibacillus polymyxa strain, Paenibacillus polymyxa strain ABP-166, a variant thereof or a derivative thereof, one or more combination(s) of the strain(s), one or more supernatant(s) from a culture of the strain(s), a cell-free fermentation product from the strain(s), or feed including one or more strain(s) or mixtures thereof. In one embodiment, the animal is a pig. In another embodiment, the animal is poultry. In yet another embodiment, the animal is a ruminant.

Administration of one or more Paenibacillus polymyxa strains, Paenibacillus polymyxa strain ABP-166, a variant thereof or a derivative thereof to animals is accomplished by any convenient method, including adding the strains to the animals' drinking water, to their feed, or to the bedding, or by direct oral insertion, such as by an aerosol or by injection.

In another embodiment, administration of one or more Paenibacillus polymyxa strains, Paenibacillus polymyxa strain ABP-166, a variant thereof or a derivative thereof is by spraying the animal with the strains. The animal can clean or preen and ingest the enzyme producing strains.

In one embodiment, the strains are administered as spores.

As used herein, the term “animal” includes, but is not limited to: human, mammal, amphibian, bird, reptile, swine, pigs, cows, cattle, goats, horses, sheep, poultry, and other animals kept or raised on a farm or ranch, sheep, big-horn sheep, buffalo, antelope, oxen, donkey, mule, deer, elk, caribou, water buffalo, camel, llama, alpaca, rabbit, mouse, rat, guinea pig, hamster, ferret, dog, cat, and other pets, primate, monkey, ape, and gorilla.

In some embodiments, the animals are birds of different ages, such as starters, growers and finishers. In certain embodiments, the animals are poultry and exotic fowl, including, but not limited to chicks, turkey poults, goslings, ducklings, guinea keets, pullets, hens, roosters (also known as cocks), cockerels, and capons.

In some embodiments, the animals are pigs, including, but not limited to: nursery pigs, breeding stock, sows, gilts, boars, lactation-phase piglets, and finishing pigs. The strain(s) can be fed to a sow during the lactation period, although the strain(s) can be fed for different durations and at different times. In certain embodiments, the strain(s) is(are) administered to piglets by feeding the strain(s) to a gilt or sow. It is believed that the transfer to the piglets from the sow is accomplished via the fecal-oral route and/or via other routes.

The Paenibacillus polymyxa strains, Paenibacillus polymyxa strain ABP-166, a variant thereof or a derivative thereof can be administered to an animal to improve at least one of nutrient digestibility, swine growth performances, poultry growth performance responses, feed efficiency (gain:feed or feed:gain), body weight, feed intake, average daily gain, average daily feed intake, the breakdown of complex dietary components, the efficiency of poultry production, the efficiency of swine production, and mortality. These benefits can be particularly useful when diets containing high levels of DDGS are fed. Initially, DDGS was from 0% to 10% of the animal's diet. Currently, DGGS is from 30% to 60%.

The amount of improvement can be measured as described herein or by other methods known in the art. These effective amounts can be administered to the animal by providing ad libitum access to feed containing the DFM. The DFM can also be administered in one or more doses.

In certain embodiments, the improvement is by at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 96%, 97%, 98%, 99%, or greater than 99% as compared to an untreated control.

In at least some embodiments, the improvement in these measurements in an animal to which the strain(s) is/are administered is at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98, 99%, and greater than 99% compared to a control animal.

In other embodiments, the improvement in these measurements in an animal to which the strain(s) is/are administered is 2-8% compared to a control animal. In certain other embodiments, the improvement in these measurements in an animal to which the strain(s) is/are administered is at least 8% compared to a control animal.

In some embodiments, a control animal is an animal that has not been administered the enzyme producing strains.

This effective amount can be administered to the animal in one or more doses. In some embodiments, the one or more Paenibacillus polymyxa strains, Paenibacillus polymyxa strain ABP-166, a variant thereof or a derivative thereof is(are) added to an animal's feed at a rate of at least 1×10⁴ CFU/animal/day.

In one embodiment, the administration improves at least one of nutrient digestibility, growth performance responses, e.g., feed efficiency, the breakdown of complex dietary components, the efficiency of production, body weight gain, feed intake, and mortality.

In certain embodiments of the method, the strain(s) is/are administered at about 1×10⁵ CFU/animal/day to about 1×10¹¹ CFU/animal/day. In some embodiments, the animal is a swine. In another embodiment, the animal is poultiy.

In at least some embodiments, the method is used when the animal is fed high levels of dried distillers grains with solubles (DDGS). The high levels of DDGS can be a rate of over 10% of the animal's diet. The high levels of DDGS can also be a rate of over 30% of the animal's diet.

In at least some embodiments, the effective amount of at least one strain of bacterium is administered to an animal by supplementing a feed intended for the animal with the effective amount of at least one strain of bacterium. As used herein, “supplementing” means the action of incorporating the effective amount of bacteria provided herein directly into the feed intended for the animal. Thus, the animal, when feeding, ingests the bacteria provided herein.

The Paenibacillus polymyxa strains, Paenibacillus polymyxa strain ABP-166, a variant thereof or a derivative thereof can be administered as a single strain or as multiple strains. Cell-free fermentation product of one or more Paenibacillus polymyxa strains, Paenibacillus polymyxa strain ABP-166, a variant thereof or a derivative thereof can be administered to an animal.

In certain embodiments, one or more Paenibacillus polymyxa strain(s), Paenibacillus polymyxa strain ABP-166, a variant thereof or a derivative thereof is (are) fed to swine. The one or more Paenibacillus polymyxa strain(s), Paenibacillus polymyxa strain ABP-166, a variant thereof or a derivative thereof address(es) the challenging components in dried distillers grains with solubles (DDGS).

In one embodiment, the Paenibacillus polymyxa strain(s), Paenibacillus polymyxa strain ABP-166, a variant thereof or a derivative thereof (s) is(are) added to animal feed at a rate of 1×10³, 1×10⁴, 1×10⁵, 1×10⁶, 1×10⁷, 1×10⁸, 1×10⁹, 1×10¹⁰, 1×10¹¹, 1×10¹², 1×10¹³ and greater than 1×10¹³ CFU per gram of animal feed.

In another embodiment, the Paenibacillus polymyxa strain(s), Paenibacillus polymyxa strain ABP-166, a variant thereof or a derivative thereof is(are) added to animal feed at a rate of 1×10³, 1×10⁴, 1×10⁵, 1×10⁶, 1×10⁷, 1×10⁸, 1×10⁹, 1×10¹⁰, 1×10¹¹, 1×10¹², 1×10¹³ and greater than 1×10¹³ CFU per animal per day.

In one embodiment, the one or more Paenibacillus polymyxa strain(s), Paenibacillus polymyxa strain ABP-166, a variant thereof or a derivative thereof is(are) added to pigs' feed at a rate of about 3.75×10⁵ CFU per gram of feed. It(they) can also be fed at about 1×10⁴ to about 1×10¹¹ CFU/animal/day. In some embodiments, the one or more Paenibacillus polymyxa strain(s), Paenibacillus polymyxa strain ABP-166, a variant thereof or a derivative thereof is(are) fed at about 1×10⁸ CFU/animal/day.

For ruminants, the one or more Paenibacillus polymyxa strain(s), Paenibacillus polymyxa strain ABP-166, a variant thereof or a derivative thereof is(are) fed at about 5×10⁹ CFU/hd/day.

For poultry, the one or more Paenibacillus polymyxa strain(s), Paenibacillus polymyxa strain ABP-166, a variant thereof or a derivative thereof is(are) added to feed at about 1×10⁴ CFU/g feed to about 1×10¹⁰ CFU/g feed. In at least some embodiments, the one or more Paenibacillus polymyxa strain(s), Paenibacillus polymyxa strain ABP-166, a variant thereof or a derivative thereof is fed at about 1×10⁵ CFU/bird/day to about 1×10⁸ CFU/bird/day.

B. Feed Material

In another embodiment, a feed for an animal comprises at least one strain of bacterium described herein. In at least some embodiments, feed is supplemented with an effective amount of at least one strain of bacterium. As used herein, “supplementing” means the action of incorporating the effective amount of bacteria provided herein directly into the feed intended for the animal. Thus, the animal, when feeding, ingests the bacteria provided herein.

When used in combination with a feed material, for monogastric diets, the feed material can include: corn, soybean meal, byproducts like distillers dried grains with solubles (DDGS), and vitamin/mineral supplement. The feed material for ruminants can be grain or hay or silage or grass, or combinations thereof. Included amongst such feed materials are: corn, dried grain, alfalfa, any feed ingredients and food or feed industry by-products as well as bio fuel industry by-products and corn meal and mixtures thereof. Other feed materials can also be used.

The time of administration can vary so long as an improvement is shown in one or more of the following: (1) breakdown of complex dietary components, (2) nutrient digestibility, (3) manure waste problems, (4) the efficiency of production, (5) carcass characteristics, (6) growth performance, (7) growth performance when feeding high levels of DDGS, (8) poultry growth performance responses, (9) swine growth performance responses, (10) the efficiency of poultry production, (11) the efficiency of swine production, (12) body weight gain, (13) feed intake, (14) feed efficiency, and (15) mortality. Administration is possible at any time with or without feed. However, the bacterium is preferably administered with or immediately before feed.

C. Methods for Improving Growth Performance of an Animal

In one embodiment, the disclosure relates to a method for improving growth performance of an animal comprising using one or more Paenibacillus polymyxa strains, Paenibacillus polymyxa strain ABP-166, a variant thereof or a derivative thereof or cell-free fermentation products therefrom to improve the growth performance of the animal relative to an animal that has not been administered the enzyme producing strains. In one embodiment, the animal is a pig. In another embodiment, the animal is poultry. In another embodiment, the animal is a ruminant.

In one embodiment, growth performance includes, but is not limited to: nutrient digestibility, poultry growth performance responses, pig growth performance responses, feed efficiency, the breakdown of complex dietary components, average daily gain, averaging daily feed intake, body weight gain, feed intake, carcass characteristics and mortality. In yet another embodiment, the methods disclosed herein are used to improve the growth performance of an animal fed an animal feed comprising DDGS.

In certain embodiments, the improvement in growth performance is by at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 96%, 97%, 98%, 99%, or greater than 99% as compared to an untreated control.

In at least some embodiments, the improvement in growth performance of an animal to which the strain(s) is/are administered is at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98, 99%, and greater than 99% compared to a control animal.

In one embodiment, the Paenibacillus polymyxa strain for improving growth performance of an animal comprise Paenibacillus polymyxa strain ABP-166, a variant thereof or a derivative thereof.

III. Anti-Contaminant Composition Comprising a Fermentation Product of Paenibacillus polymyxa

An anti-contaminant composition comprising a fermentation product of Paenibacillus polymyxa strains has inhibitory activity against microorganisms, including harmful bacteria. In one embodiment, the anti-contaminant composition comprises a fermentation product of Paenibacillus polymyxa ABP-166.

In at least some embodiments, the anti-contaminant composition is a cell-free fermentation product of Paenibacillus polymyxa. In other embodiments, the anti-contaminant composition is not cell-free.

In at least some embodiments, the anti-contaminant composition is a cell-free fermentation product of Paenibacillus polymyxa ABP-166. In other embodiments, the anti-contaminant composition is not cell-free.

In certain embodiments, a cell-free fermentation product of a Paenibacillus polymyxa strain, Paenibacillus polymyxa strain ABP-166, a variant thereof or a derivative thereof is used in a w/v percent, including but not limited to at least 1%, 5%, 10%, 15%, 20%, 25%, 30%, 35%, 40%, 45%, 50%, 55%, 60%, 65%, 70%, 75%, 80%, 85%, 90%, or 95%.

In one embodiment, an anti-contaminant composition comprising a cell-free fermentation product of a Paenibacillus polymyxa strain, Paenibacillus polymyxa strain ABP-166, a variant thereof or a derivative thereof is active against gram negative bacteria, including but not limited to Bordetella spp., Enterobacter spp., Erwinia spp., E. coli O157, Hafnia spp., Klebsiella spp., Proteus spp., Pseudomonas spp., Salmonella spp., Shewanella spp., Vibrio spp., Pantoea agglomerans, Buttiauxella agrestis, Carnobacterium spp., Yersinia aldovae, Bacillus subtilis, Shewanella putrefaciens, Aeromonas salmonicida, Carnobacterium divergens, Yersinia frederiksenii, Aeromonas veronii bv. Sobria, Enterobacter kobei, Staphylococcus pasteuri, Citrobacter freundii, Stenotrophomonas maltophilia, Yersinia spp., Pseudoalternomonas spp., Hafnia alvei, Aeromonas spp., and Yersinia enterocolitica.

In one embodiment, an anti-contaminant composition comprising a cell-free fermentation product of a Paenibacillus polymyxa strain, Paenibacillus polymyxa strain ABP-166, a variant thereof or a derivative thereof is active against gram positive bacteria including but not limited to Alicyclobacillus spp., Clostridium spp., Lactococcous spp., Leoconostoc spp., Staphylococcus spp., and Listeria monocytogenes.

Table 1 provides a summary of microorganisms that are inhibited by Paenibacillus polymyxa strain ABP166, or its fermentation derivative, and the applications in which the strain was applied. As shown in Table 1, numerous microbial isolates were tested, demonstrating wide applicability of the inhibitory activity. Paenibacillus polymyxa strain ABP166, or its fermentation derivative showed inhibitory activity against a wide range of bacteria as well as isolates for a particular species. For example, over 40 different isolates of E. coli were inhibited by Paenibacillus polymyxa strain ABP166, or its fermentation derivative, which demonstrates a broad spectrum inhibitory activity.

TABLE 1
Summary of microorganisms with demonstrated inhibition by
Paenibacillus polymyxa strain ABP166 or its fermentation
product derivative.

| | | Inhibition by | Application |
|---|---|---|---|
| Microbial Isolate | Identity | ABP166, % | Field |
| Escherichia coli | Gram negative bacteria | 99.14 | Fresh meat |
| Staphylococcus spp. | Gram positive bacteria | 93.71 | Fresh meat |
| Pseudomonas spp. | Gram negative bacteria | 98.99 | Fresh meat |
| Pantoea agglomerans | Gram negative bacteria | 94.76 | Fresh meat |
| Buttiauxella agrestis | Gram negative bacteria | 99.16 | Fresh meat |
| Carnobacterium spp. | Gram negative bacteria | 95.81 | Fresh meat |
| Shewanella spp. | Gram negative bacteria | 99.82 | Fresh meat |
| Yersinia aldovae | Gram negative bacteria | 99.68 | Fresh meat |
| Bacillus subtilis | Gram positive bacteria | 67.68 | Fresh meat |
| Shewanella putrefaciens | Gram negative bacteria | 95.77 | Fresh meat |
| Aeromonas salmonicida | Gram negative bacteria | 98.89 | Fresh meat |
| Yersinia enterocolitica | Gram negative bacteria | 97.72 | Fresh meat |
| Enterobacter spp. | Gram negative bacteria | 99.22 | Fresh meat |
| Carnobacterium divergens | Gram negative bacteria | 98.10 | Fresh meat |
| Yersinia frederiksenil | Gram negative bacteria | 98.03 | Fresh meat |
| Aeromonas veronii bv. | Gram negative bacteria | 98.97 | Fresh meat |
| Sobria | | | |
| Pantoea agglomerans | Gram negative bacteria | 99.31 | Fresh meat |
| Enterobacter kobei | Gram negative bacteria | 99.09 | Fresh meat |
| Staphylococcus pasteuri | Gram positive bacteria | 98.66 | Fresh meat |
| Citrobacter freundii | Gram negative bacteria | 73.20 | Fresh meat |
| Pseudomonas spp. | Gram negative bacteria | 99.42 | Fresh meat |
| Carnobacterium spp. | Gram negative bacteria | 97.17 | Fresh meat |
| Stenotrophomonas | Gram negative bacteria | 99.40 | Fresh meat |
| maltophilia | | | |
| Yersinia spp. | Gram negative bacteria | 97.35 | Fresh meat |
| Pseudoalteromonas spp. | Gram negative bacteria | 99.52 | Fresh meat |
| Shewanella putrefaciens | Gram negative bacteria | 91.82 | Fresh meat |
| Shewanella putrefaciens | Gram negative bacteria | 99.76 | Fresh meat |
| Yersinia spp. | Gram negative bacteria | 98.63 | Fresh meat |
| Hafnia alvei | Gram negative bacteria | 92.80 | Fresh meat |
| Carnobacterium divergens | Gram negative bacteria | 92.10 | Fresh meat |
| Aeromonas spp. | Gram negative bacteria | 99.47 | Fresh meat |
| Yersinia frederiksenil | Gram negative bacteria | 97.92 | Fresh meat |
| Buttiauxella agrestis | Gram negative bacteria | 99.60 | Fresh meat |
| Alicyclobacillus spp. | Gram positive bacteria | 100 | Fruit juice |
| Alicyclobacillus | Gram positive bacteria | 100 | Fruit juice |
| acidoterrestris | | | |
| Alicyclobacillus | Gram positive bacteria | 100 | Food |
| acidoterrestris | | | |
| Alicyclobacillus spp. | Gram positive bacteria | 100 | Fruit juice |
| Alicyclobacillus | Gram positive bacteria | 99.3 | Fruit juice |
| acidoterrestris | | | |
| Alicyclobacillus spp. | Gram positive bacteria | 98.2 | Fruit juice |
| Alicyclobacillus spp. | Gram positive bacteria | 100 | Fruit juice |
| Alicyclobacillus spp. | Gram positive bacteria | 97.6 | Fruit juice |
| Alicyclobacillus spp. | Gram positive bacteria | 98.8 | Fruit juice |
| Bacillus amyloliquefaciens | Gram positive bacteria | 100 | Fresh meat |
| Enterococcus hirae | Gram positive bacteria | 100 | Fresh meat |
| Staphylococcus cohnii | Gram positive bacteria | 30 | Fresh meat |
| Bacillus pumilus | Gram positive bacteria | 100 | Fresh meat |
| Corynebacterium jeikeium | Bacteria | 7 | Fresh meat |
| Micrococcus caseolyticus | Bacteria | 100 | Fresh meat |
| Bacillus amyloliquefaciens | Gram positive bacteria | 98 | Fresh meat |
| Staphylococcus lentus | Gram positive bacteria | 98 | Fresh meat |
| Bacillus licheniformis | Gram positive bacteria | 50 | Fresh meat |
| Bacillus licheniformis | Gram positive bacteria | 57 | Fresh meat |
| Bacillus thuringiensis | Gram positive bacteria | 99 | Fresh meat |
| Yersinia enterocolitica | Gram negative bacteria | 97 | Fresh meat |
| Bacillus amyloliquefaciens | Gram positive bacteria | 100 | Fresh meat |
| Enterobacter spp. | Gram negative bacteria | 90 | Fresh meat |
| Enterobacter spp. | Gram negative bacteria | 69 | Fresh meat |
| Rahnella aquatilis | Gram negative bacteria | 96 | Fresh meat |
| Listeria monocytogenes | Gram positive bacteria | 70 | Fresh meat |
| Enterobacter sakazakii | Gram negative bacteria | 89.1 | Infant formula |
| Leuconostoc garlicum | Gram positive bacteria | 100 | Cut flowers |
| Lactococcous lactis | Gram positive bacteria | 99 | Cut flowers |
| Erwinia crysanthemi | Gram negative bacteria | 95 | Cut flowers |
| Leuconostoc lactis | Gram positive bacteria | 97 | Cut flowers |
| Erwinia herbicola | Gram negative bacteria | 93 | Cut flowers |
| Leuconostoc garlicum | Gram positive bacteria | 100 | Cut flowers |
| Bacillus amyloliquifaciens | Gram positive bacteria | 99.8 | Cut flowers |
| Erwinia herbicola | Gram negative bacteria | 100 | Cut flowers |
| Erwinia herbicola | Gram negative bacteria | 100 | Cut flowers |
| Erwinia herbicola | Gram negative bacteria | 100 | Cut flowers |
| Pantoea sp. | Gram negative bacteria | 44.39 | Cut flowers |
| Raoutella terrigena | Gram negative bacteria | 83.93 | Cut flowers |
| Pseudomonas | Gram negative bacteria | 91.26 | Cut flowers |
| plecoglossicida | | | |
| Bacillus sp. | Gram positive bacteria | 54.81 | Cut flowers |
| Serratia sp. | Gram negative bacteria | 90.47 | Cut flowers |
| Weissella soli | Gram positive bacteria | 7.40 | Cut flowers |
| Pantoea septic | Gram negative bacteria | 87.56 | Cut flowers |
| Pseudomonas | Gram negative bacteria | 59.21 | Cut flowers |
| extremaustralis | | | |
| Pseudomonas sp. | Gram negative bacteria | 0.64 | Cut flowers |
| Pantoea sp. | Gram negative bacteria | 85.94 | Cut flowers |
| Erwinia Billingae | Gram negative bacteria | 85.53 | Cut flowers |
| Enterobacter sp. | Gram negative bacteria | 92.37 | Cut flowers |
| Yersinia sp. | Gram negative bacteria | 92.39 | Cut flowers |
| Kluyvera intermedia | Gram negative bacteria | 27.19 | Cut flowers |
| Enterobacter ludwigii | Gram negative bacteria | 31.79 | Cut flowers |
| Pseudomonas koreensis | Gram negative bacteria | 52.99 | Cut flowers |
| Serratia sp. | Gram negative bacteria | 65.49 | Cut flowers |
| Pantoea sp. | Gram negative bacteria | 70.48 | Cut flowers |
| Erwinia sp. | Gram negative bacteria | 90.38 | Cut flowers |
| Pantoea sp. | Gram negative bacteria | 68.46 | Cut flowers |
| Pantoea sp. | Gram negative bacteria | 78.98 | Cut flowers |
| Pseudomonas sp. | Gram negative bacteria | 0.00 | Cut flowers |
| Pseudomonas montelii | Gram negative bacteria | 0.00 | Cut flowers |
| Pseudomonas veronii | Gram negative bacteria | 19.75 | Cut flowers |
| Pseudomonas veronii | Gram negative bacteria | 30.54 | Cut flowers |
| Pantoea ananatis | Gram negative bacteria | 93.40 | Cut flowers |
| Pseudomonas sp. | Gram negative bacteria | 90.61 | Cut flowers |
| Pseudomonas lurida | Gram negative bacteria | 67.68 | Cut flowers |
| Pseudomonas abietaniphila | Gram negative bacteria | 86.64 | Cut flowers |
| Pantoea sp. | Gram negative bacteria | 76.09 | Cut flowers |
| Pseudomonas koreensis | Gram negative bacteria | 93.51 | Cut flowers |
| Pseudomonas sp. | Gram negative bacteria | 86.82 | Cut flowers |
| Pseudomonas lurida | Gram negative bacteria | 89.75 | Cut flowers |
| Pseudomonas graminis | Gram negative bacteria | 0.00 | Cut flowers |
| Pseudomonas libaniensis | Gram negative bacteria | 35.54 | Cut flowers |
| Rahnella aquatilis | Gram negative bacteria | 86.86 | Cut flowers |
| Pseudomonas sp. | Gram negative bacteria | 76.50 | Cut flowers |
| Pseudomonas veronii | Gram negative bacteria | 51.09 | Cut flowers |
| Pseudomonas abietaniphila | Gram negative bacteria | 87.51 | Cut flowers |
| Pseadomonas fragi | Gram negative bacteria | 85.13 | Cut flowers |
| Pseudomonas sp. | Gram negative bacteria | 14.91 | Cut flowers |
| Enterobacter amnigenus | Gram negative bacteria | 81.95 | Cut flowers |
| Pantoea sp. | Gram negative bacteria | 85.39 | Cut flowers |
| Pseudomonas koreensis | Gram negative bacteria | 92.16 | Cut flowers |
| Pseudomonas koreensis | Gram negative bacteria | 87.48 | Cut flowers |
| Curtobacterium sp. | Gram positive bacteria | 82.86 | Cut flowers |
| Curtobacterium sp. | Gram positive bacteria | 88.65 | Cut flowers |
| Stenotrophomonas | Gram negative bacteria | 86.55 | Cut flowers |
| rhizosphaerae | | | |
| Pseudomonas sp. | Gram negative bacteria | 94.28 | Cut flowers |
| Pseudomonas flectens | Gram negative bacteria | 67.68 | Cut flowers |
| Pseudomonas azotoformans | Gram negative bacteria | 92.39 | Cut flowers |
| Pseudomonas constantinii | Gram negative bacteria | 35.03 | Cut flowers |
| Pseudomonas constantinii | Gram negative bacteria | 0.00 | Cut flowers |
| Bacillus methylotrophicus | Gram positive bacteria | 54.63 | Cut flowers |
| Pseudomonas | Gram negative bacteria | 69.80 | Cut flowers |
| psychrotolerans | | | |
| Enterobacter amnigenus | Gram negative bacteria | 90.23 | Cut flowers |
| Pseudomonas sp. | Gram negative bacteria | 0.00 | Cut flowers |
| Pseudomonas | Gram negative bacteria | 0.00 | Cut flowers |
| plecoglossicida | | | |
| Pantoea sp. | Gram negative bacteria | 94.38 | Cut flowers |
| Raoutella terrigena | Gram negative bacteria | 89.55 | Cut flowers |
| Pseudomonas | Gram negative bacteria | 88.41 | Cut flowers |
| extremorientalis | | | |
| Vibrio parahaemolyticus | Gram negative bacteria | + | Aquaculture |
| Vibrio harveyi | Gram negative bacteria | + | Aquaculture |
| Vibrio vulnificus | Gram negative bacteria | + | Aquaculture |
| Vibrio ponticus | Gram negative bacteria | + | Aquaculture |
| Escherichia coli | Gram negative bacteria | 99.6 | Poultry |
| Escherichia coli | Gram negative bacteria | 99.5 | Poultry |
| Escherichia coli | Gram negative bacteria | 99.7 | Poultry |
| Escherichia coli | Gram negative bacteria | 99.5 | Poultry |
| Escherichia coli | Gram negative bacteria | 99.7 | Poultry |
| Escherichia coli | Gram negative bacteria | 99.8 | Poultry |
| Escherichia coli | Gram negative bacteria | 99.6 | Poultry |
| Escherichia coli | Gram negative bacteria | 86.6 | Poultry |
| Escherichia coli | Gram negative bacteria | 99.7 | Poultry |
| Escherichia coli | Gram negative bacteria | 99.8 | Poultry |
| Escherichia coli | Gram negative bacteria | 55.6 | Poultry |
| Escherichia coli | Gram negative bacteria | 99.3 | Poultry |
| Escherichia coli | Gram negative bacteria | 99.7 | Poultry |
| Escherichia coli | Gram negative bacteria | 99.7 | Poultry |
| Escherichia coli | Gram negative bacteria | 85.9 | Poultry |
| Escherichia coli | Gram negative bacteria | 97.1 | Poultry |
| Escherichia coli | Gram negative bacteria | 98.0 | Poultry |
| Escherichia coli | Gram negative bacteria | 64.2 | Poultry |
| Escherichia coli | Gram negative bacteria | 94.3 | Poultry |
| Escherichia coli | Gram negative bacteria | 92.2 | Poultry |
| Escherichia coli | Gram negative bacteria | 94.4 | Poultry |
| Escherichia coli | Gram negative bacteria | 93.6 | Poultry |
| Escherichia coli | Gram negative bacteria | 99.6 | Poultry |
| Escherichia coli | Gram negative bacteria | 99.7 | Poultry |
| Escherichia coli | Gram negative bacteria | 99.1 | Poultry |
| Escherichia coli | Gram negative bacteria | 99.6 | Poultry |
| Escherichia coli | Gram negative bacteria | 100.0 | Poultry |
| Escherichia coli | Gram negative bacteria | 99.6 | Poultry |
| Escherichia coli | Gram negative bacteria | 100.0 | Poultry |
| Escherichia coli | Gram negative bacteria | 100.0 | Poultry |
| Escherichia coli | Gram negative bacteria | 95.1 | Poultry |
| Escherichia coli | Gram negative bacteria | 99.8 | Poultry |
| Escherichia coli | Gram negative bacteria | 99.5 | Poultry |
| Escherichia coli | Gram negative bacteria | 99.7 | Poultry |
| Escherichia coli | Gram negative bacteria | 99.9 | Poultry |
| Escherichia coli | Gram negative bacteria | 98.5 | Poultry |
| Escherichia coli | Gram negative bacteria | 99.7 | Poultry |
| Escherichia coli | Gram negative bacteria | 99.8 | Poultry |
| Escherichia coli | Gram negative bacteria | 99.5 | Poultry |
| Escherichia coli | Gram negative bacteria | 94.8 | Poultry |
| Escherichia coli | Gram negative bacteria | 99.6 | Poultry |
| Escherichia coli | Gram negative bacteria | 95.5 | Poultry |
| Escherichia coli | Gram negative bacteria | 99.8 | Poultry |
| Clostridium perfringens | Gram positive bacteria | 99.31 | Poultry |
| Clostridium perfringens | Gram positive bacteria | 99.19 | Poultry |
| Clostridium perfringens | Gram positive bacteria | 100.00 | Poultry |
| Clostridium perfringens | Gram positive bacteria | 99.63 | Poultry |
| Clostridium perfringens | Gram positive bacteria | 100.00 | Poultry |
| Clostridium perfringens | Gram positive bacteria | 94.93 | Poultry |
| Clostridium perfringens | Gram positive bacteria | 95.74 | Poultry |
| Clostridium perfringens | Gram positive bacteria | 99.63 | Poultry |
| Clostridium perfringens | Gram positive bacteria | 98.88 | Poultry |
| Clostridium perfringens | Gram positive bacteria | 99.91 | Poultry |
| Clostridium perfringens | Gram positive bacteria | 98.79 | Poultry |
| Clostridium perfringens | Gram positive bacteria | 93.70 | Poultry |
| Clostridium perfringens | Gram positive bacteria | 97.46 | Poultry |
| Clostridium perfringens | Gram positive bacteria | 98.30 | Poultry |
| Clostridium perfringens | Gram positive bacteria | 99.28 | Poultry |
| Clostridium perfringens | Gram positive bacteria | 99.84 | Poultry |
| Clostridium perfringens | Gram positive bacteria | 99.43 | Poultry |
| Clostridium perfringens | Gram positive bacteria | 99.48 | Poultry |
| Clostridium perfringens | Gram positive bacteria | 100.00 | Poultry |
| Clostridium perfringens | Gram positive bacteria | 98.48 | Poultry |
| Clostridium perfringens | Gram positive bacteria | 99.96 | Poultry |
| Clostridium perfringens | Gram positive bacteria | 98.32 | Poultry |
| Clostridium perfringens | Gram positive bacteria | 97.68 | Poultry |
| Clostridium perfringens | Gram positive bacteria | 98.15 | Poultry |
| Clostridium perfringens | Gram positive bacteria | 17.78 | Poultry |
| Clostridium perfringens | Gram positive bacteria | 40.52 | Poultry |
| Clostridium perfringens | Gram positive bacteria | 99.45 | Poultry |
| Clostridium perfringens | Gram positive bacteria | 98.91 | Poultry |
| Clostridium perfringens | Gram positive bacteria | 99.88 | Poultry |
| Clostridium perfringens | Gram positive bacteria | 97.58 | Poultry |
| Clostridium perfringens | Gram positive bacteria | 99.25 | Poultry |
| Clostridium perfringens | Gram positive bacteria | 99.58 | Poultry |
| Salmonella spp. | Gram negative bacteria | 87.09 | Poultry |
| Salmonella spp. | Gram negative bacteria | 89.17 | Poultry |
| Salmonella spp. | Gram negative bacteria | 91.16 | Poultry |
| Salmonella spp. | Gram negative bacteria | 91.38 | Poultry |
| Salmonella spp. | Gram negative bacteria | 91.49 | Poultry |
| Salmonella spp. | Gram negative bacteria | 91.55 | Poultry |
| Salmonella spp. | Gram negative bacteria | 91.55 | Poultry |
| Salmonella spp. | Gram negative bacteria | 91.57 | Poultry |
| Salmonella spp. | Gram negative bacteria | 91.59 | Poultry |
| Salmonella spp. | Gram negative bacteria | 91.87 | Poultry |
| Salmonella spp. | Gram negative bacteria | 91.96 | Poultry |
| Salmonella spp. | Gram negative bacteria | 92.00 | Poultry |
| Salmonella spp. | Gram negative bacteria | 92.25 | Poultry |
| Salmonella spp. | Gram negative bacteria | 92.73 | Poultry |
| Salmonella spp. | Gram negative bacteria | 92.79 | Poultry |
| Salmonella spp. | Gram negative bacteria | 92.99 | Poultry |
| Salmonella spp. | Gram negative bacteria | 93.10 | Poultry |
| Salmonella spp. | Gram negative bacteria | 93.26 | Poultry |
| Salmonella spp. | Gram negative bacteria | 93.26 | Poultry |
| Salmonella spp. | Gram negative bacteria | 93.46 | Poultry |
| Salmonella spp. | Gram negative bacteria | 93.91 | Poultry |
| Salmonella spp. | Gram negative bacteria | 93.96 | Poultry |
| Salmonella spp. | Gram negative bacteria | 94.16 | Poultry |
| E. coli O157 | Gram negative bacteria | 91.92 | Cattle |
| E. coli O157 | Gram negative bacteria | 91.00 | Cattle |
| E. coli O157 | Gram negative bacteria | 91.26 | Cattle |
| E. coli O157 | Gram negative bacteria | 93.93 | Cattle |
| E. coli O157 | Gram negative bacteria | 91.18 | Cattle |
| E. coli O157 | Gram negative bacteria | 92.29 | Cattle |
| E. coli O157 | Gram negative bacteria | 92.61 | Cattle |
| E. coli O157 | Gram negative bacteria | 92.09 | Cattle |
| E. coli O157 | Gram negative bacteria | 92.37 | Cattle |
| E. coli O157 | Gram negative bacteria | 92.50 | Cattle |
| E. coli O157 | Gram negative bacteria | 93.44 | Cattle |
| E. coli O157 | Gram negative bacteria | 93.65 | Cattle |
| E. coli O157 | Gram negative bacteria | 92.86 | Cattle |
| E. coli O157 | Gram negative bacteria | 92.94 | Cattle |
| E. coli O157 | Gram negative bacteria | 93.50 | Cattle |
| E. coli O157 | Gram negative bacteria | 93.45 | Cattle |
| E. coli O157 | Gram negative bacteria | 93.14 | Cattle |
| E. coli O157 | Gram negative bacteria | 93.78 | Cattle |
| E. coli O157 | Gram negative bacteria | 94.18 | Cattle |
| E. coli O157 | Gram negative bacteria | 92.44 | Cattle |
| E. coli O157 | Gram negative bacteria | 93.22 | Cattle |
| E. coli O157 | Gram negative bacteria | 92.70 | Cattle |
| E. coli O157 | Gram negative bacteria | 93.20 | Cattle |
| E. coli O157 | Gram negative bacteria | 93.48 | Cattle |
| E. coli O157 | Gram negative bacteria | 93.36 | Cattle |
| E. coli O157 | Gram negative bacteria | 92.90 | Cattle |
| E. coli O157 | Gram negative bacteria | 92.84 | Cattle |
| E. coli O157 | Gram negative bacteria | 93.06 | Cattle |
| E. coli O157 | Gram negative bacteria | 92.01 | Cattle |
| E. coli O157 | Gram negative bacteria | 92.95 | Cattle |
| E. coli O157 | Gram negative bacteria | 93.61 | Cattle |
| Salmonella Spp. | Gram negative bacteria | 92.89 | Cattle |
| Salmonella Spp. | Gram negative bacteria | 93.05 | Cattle |
| Salmonella Spp. | Gram negative bacteria | 91.61 | Cattle |
| Salmonella Spp. | Gram negative bacteria | 90.65 | Cattle |
| Salmonella. derby | Gram negative bacteria | 94.21 | Cattle |
| Salmonella. agona | Gram negative bacteria | 93.14 | Cattle |
| Salmonella. montevideo | Gram negative bacteria | 94.25 | Cattle |
| E. coli | Gram negative bacteria | 50.85 | Feline |
| E. coli | Gram negative bacteria | 67.70 | Feline |
| E. coli | Gram negative bacteria | 48.85 | Feline |
| E. coli | Gram negative bacteria | 20.11 | Feline |
| E. coli | Gram negative bacteria | 48.85 | Feline |
| E. coli | Gram negative bacteria | 58.09 | Feline |
| Escherichia spp. | Gram negative bacteria | 42.07 | Feline |
| E. coli | Gram negative bacteria | 57.92 | Feline |
| E. coli | Gram negative bacteria | 82.14 | Feline |
| Enterococcus faecium | Gram positive bacteria | 88.28 | Feline |
| Enterococcus faecium | Gram positive bacteria | 88.53 | Feline |
| Enterococcus faecium | Gram positive bacteria | 87.96 | Feline |
| Enterococcus hirae | Gram positive bacteria | 67.80 | Feline |
| Staphylococcus aureus | Gram positive bacteria | 84.64 | Feline |
| Staphylococcus aureus | Gram positive bacteria | 85.04 | Feline |
| Staphylococcus aureus | Gram positive bacteria | 87.06 | Feline |
| Escherichia spp. | Gram negative bacteria | 45.03 | Feline |
| Staphylococcus spp. | Gram positive bacteria | 62.64 | Feline |
| Micrococcus luteus | Bacteria | 74.40 | Feline |
| E. coli | Gram negative bacteria | 45.44 | Feline |
| E. coli | Gram negative bacteria | 60.61 | Feline |
| E. coli | Gram negative bacteria | 60.84 | Feline |
| E. coli | Gram negative bacteria | 58.24 | Feline |
| E. coli | Gram negative bacteria | 72.51 | Feline |
| E. coli | Gram negative bacteria | 46.71 | Feline |
| E. coli | Gram negative bacteria | 44.55 | Feline |
| E. coli | Gram negative bacteria | 40.15 | Feline |
| E. coli | Gram negative bacteria | 33.68 | Feline |
| Bacillus cereus | Gram positive bacteria | + | Food |
| Bacillus cereus (spores) | Gram positive bacteria | + | Food |
| Bacillus licheniformis | Gram positive bacteria | + | Food |
| Bacillus licheniformis | Gram positive bacteria | + | Food |
| (spores) | | | |
| Bacillus | Gram positive bacteria | + | Food |
| weihenstephanensis | | | |
| Bacillus | Gram positive bacteria | + | Food |
| weihenstephanensis | | | |
| Listeria monocytogenes | Gram positive bacteria | + | Food |
| Listeria monocytogenes | Gram positive bacteria | + | Food |
| Listeria innocua | Gram positive bacteria | + | Food |
| Lactobacillus fermentum | Gram positive bacteria | + | Food |
| Lactobacilllus curvatus | Gram positive bacteria | + | Food |
| Lactobacilllus sakei | Gram positive bacteria | + | Food |
| Lactobacillus plantarum | Gram positive bacteria | + | Food |
| Leuconostoc mesenteroides | Gram positive bacteria | + | Food |
| ss | | | |
| Clostridium sporogenes | Gram positive bacteria | + | Food |
| Clostridium sporogenes | Gram positive bacteria | + | Food |
| Clostridium sporogenes | Gram positive bacteria | + | Food |
| Clostridium sporogenes | Gram positive bacteria | + | Food |
| (spores) | | | |
| Clostridium sporogenes | Gram positive bacteria | + | Food |
| (spores) | | | |
| Clostridium sporogenes | Gram positive bacteria | + | Food |
| (spores) | | | |
| Escherichia coli | Gram negative bacteria | + | Food |
| Escherichia coli | Gram negative bacteria | + | Food |
| Escherichia coli O157:H7 | Gram negative bacteria | + | Food |
| Klebsiella oxytoca | Gram negative bacteria | + | Food |
| Citrobacter freundii | Gram negative bacteria | + | Food |
| Pseudomonas putida | Gram negative bacteria | + | Food |
| Salmonella typhimurium | Gram negative bacteria | + | Food |
| Salmonella typhimurium | Gram negative bacteria | + | Food |
| Hafnia alvei | Gram negative bacteria | + | Food |
| Saccharomyces cerevisiae | Yeast | + | Food |
| Zygosaccharomyces bailii | Yeast | + | Food |
| Pichia anomala | Yeast | + | Food |
| Klyveromyces marxianus | Yeast | + | Food |
| Candida parapsilosis | Yeast | + | Food |
| Candida tropicalis | Yeast | + | Food |
| Penicillium commune | Mold | + | Food |
| Aspergillus versicolor | Mold | + | Food |
| Aspergillus parasiticus | Mold | + | Food |
| Salmonella enterica | Gram negative bacteria | 86.79 | Pet food |
| Salmonella enterica | Gram negative bacteria | 88.69 | Pet food |
| Salmonella enterica | Gram negative bacteria | 90.08 | Pet food |
| Salmonella enterica | Gram negative bacteria | 88.15 | Pet food |
| Salmonella enterica | Gram negative bacteria | 92.23 | Pet food |
| Salmonella enterica | Gram negative bacteria | 92.29 | Pet food |
| Salmonella enterica | Gram negative bacteria | 93.86 | Pet food |
| Salmonella enterica | Gram negative bacteria | 92.40 | Pet food |
| Salmonella enterica | Gram negative bacteria | 90.24 | Pet food |
| Salmonella enterica | Gram negative bacteria | 92.82 | Pet food |
| Salmonella enterica | Gram negative bacteria | 92.06 | Pet food |
| Salmonella enterica | Gram negative bacteria | 92.40 | Pet food |
| Salmonella enterica | Gram negative bacteria | 93.13 | Pet food |
| Salmonella enterica | Gram negative bacteria | 93.09 | Pet food |
| Salmonella enterica | Gram negative bacteria | 90.58 | Pet food |
| Salmonella enterica | Gram negative bacteria | 93.07 | Pet food |
| Salmonella species | Gram negative bacteria | 92.85 | Pet food |
| Salmonella enterica | Gram negative bacteria | 87.94 | Pet food |
| Salmonella enterica | Gram negative bacteria | 93.31 | Pet food |
| Salmonella enterica | Gram negative bacteria | 91.50 | Pet food |
| Salmonella enterica | Gram negative bacteria | 93.06 | Pet food |
| Salmonella enterica | Gram negative bacteria | 92.81 | Pet food |
| Salmonella enterica | Gram negative bacteria | 89.82 | Pet food |
| Salmonella enterica | Gram negative bacteria | 90.10 | Pet food |
| Salmonella enterica | Gram negative bacteria | 93.40 | Pet food |
| Salmonella enterica | Gram negative bacteria | 92.08 | Pet food |
| Salmonella enterica | Gram negative bacteria | 94.18 | Pet food |
| Salmonella enterica | Gram negative bacteria | 93.72 | Pet food |
| Salmonella enterica | Gram negative bacteria | 84.36 | Pet food |

The cell-free fermentation product of ABP-166 had good pH tolerance, with activity against both E. coli O51 and Listeria, at pHs between 1.0 and 9.0. In addition, the cell-free fermentation product was stable following desiccation. This provides the ability to spray dry the cell-free fermentation product for desired uses. The Listeria genus includes seven species including Listeria monocytogenes, which causes listeriosis, a serious infection caused by eating food contaminated by the bacteria. The overt form of the disease has a mortality rate of 20%.

The activity of the cell-free fermentation product of ABP-166 was stable at −20° C. with activity remaining through nine months of testing. A freeze-thaw assay at −20° C. was also run for the cell-free fermentation product of ABP-166 with indicator organisms E. coli O157 and Listeria. Activity remained through nine months of testing.

The activity of the cell-free fermentation product of ABP-166 was analyzed in a commercial optimization efficacy assay. The activity of cell-free fermentation product of ABP-166 was also tested for inhibitory activity against E. coli O157 with increasing concentrations of the cell-free fermentation product. In about 30 minutes, a 10% solution of the cell-free fermentation product produced a 0.5 log reduction in E. coli O157, a 50% the cell-free fermentation product produced a 5 log reduction, and a 100% solution of the cell-free fermentation product produced a 7 log reduction.

In addition, cell-free fermentation product was tested to determine whether E. coli O157 isolates acquired resistance. For this, two E. coli O157 isolates were treated with 10% solution of the cell-free fermentation product. A control lacking the cell-free fermentation product showed increasing growth of E. coli, whereas the sample treated with the fermentation product reduced the level of E. coli down to the detection limit. After 24 hours of growth, the cell-free fermentation product sample was clear and showed no growth, whereas the control sample not treated with the cell-free fermentation product was turbid with growth of E. coli.

E. coli O157 treated with a 10% solution of cell-free fermentation product found over 95% inhibition in only five minutes. Additional pathogens tested with the cell-free fermentation product included planktonic Salmonella enterica sv. Newport. A 10% solution of the cell-free fermentation product produced a greater than 4 log reduction after 200 minutes of treatment.

A similar assay was run using planktonic L. monocytogenes using a 10% solution of the cell-free fermentation product. After about 200 minutes, there was over a 4 log reduction in the planktonic L. monocytogenes culture.

### IV. Contaminant Microorganisms

In one aspect, the contaminant microorganisms may be a Gram-negative bacterium, a Gram-positive bacterium, or a fungus. In some aspects, the contaminant microorganisms may be a plurality of microorganisms, e.g., microorganisms selected from the group consisting of: Gram-negative bacteria, Gram-positive bacteria, and fungi.

In another aspect, the contaminant microorganisms may be one or more Gram-negative bacteria from a genus selected from the group consisting of: Salmonella; Escherichia; Hafnia; Klebsiella; Pseudomonas; Shigella; and Yersinia.

In one aspect, the contaminant microorganisms may be one or more of: Salmonella enterica; Escherichia coli; Hafnia alvei; Klebsiella oxytoca; Pseudomonas fluorescens; Pseudomonas putida; Salmonella typhimurium; Shigella flexneri; Shigella sonnei; and Yersinia enterocolitica.

In one aspect, a composition disclosed herein is effective against a Salmonella enterica strain.

In one embodiment, the contaminant microorganisms may be selected from one or more of: Salmonella enterica ser. Anatum; Salmonella enterica ser. Braenderup; Salmonella enterica ser. Derby; Salmonella enterica ser. Enteritidis; Salmonella enterica ser. Hadar, Salmonella enterica ser. Infantis; Salmonella enterica ser. Kedougou; Salmonella enterica ser. Mbandaka; Salmonella enterica ser. Montevideo; Salmonella enterica ser. Neumuenster; Salmonella enterica ser. Newport; Salmonella enterica ser. Ohio; Salmonella enterica ser. Schwarzengrund; Salmonella enterica ser. Senftenberg; Salmonella enterica ser. Tennessee; Salmonella enterica ser. Thompson; and Salmonella enterica ser. Typhimurium.

In one embodiment, the contaminant microorganism may be Escherichia.

In another embodiment, the contaminant microorganism may be Escherichia coli.

In yet another embodiment, the contaminant microorganisms may be selected from one or more of: E. coli DCS 15 (e.g. E. coli O157:H7), E. coli DCS 492, E. coli DCS 493, E. coli DCS 494, E. coli DCS 495, E. coli DCS 496, E. coli DCS 497, E. coli DCS 546, E. coli DCS 558, E. coli DCS 1336, and E. coli DCS 1396.

In still another embodiment, the contaminant microorganisms may be one or more Gram-positive bacteria from a genus selected from the group consisting of: Listeria; Bacillus; Brochothrix; Clostridium; Enterococcus; Lactobacillus; Leuconostoc; and Staphylococcus.

In another embodiment, the contaminant microorganisms may be one or more of: Listeria monocytogenes; Bacillus coagulans spores; Bacillus licheniformis; Bacillus licheniformis spores; Bacillus subtilis spores; Brochothrix thermosphacta; Clostridium perfringens; Clostridium sporogenes spores; Enterococcus faecalis; Enterococcus gallinarum; Lactobacillus farciminis; Lactobacillus fermentum; Lactobacillus plantarum; Lactobacillus sakei; Leuconostoc mesenteroides; Listeria innocua; Staphylococcus aureus; and Staphylococcus epidermidis.

In yet another embodiment, the contaminant microorganisms may be one or more fungi from a genus selected from the group consisting of: Aspergillus; Candida; Debaryomyces; Kluyveromyces; Penicillium; Pichia; Rhodotorula; Saccharomyces; and Zygosaccharomyces.

In one aspect, the contaminant microorganisms may be one or more of: Aspergillus parasiticus; Aspergillus versicolor; Candida parapsilosis; Candida tropicalis; Citrobacter freundii; Debaryomyces hansenii; Kluyveromyces marxianus; Penicillium commune; Pichia anomala; Rhodotorula glutinis; Rhodotorula mucilaginosa; Saccharomyces cerevisiae; and Zygosaccharomyces bailii.

Examples of Gram-positive contaminant microorganisms include bacteria from the genera: Listeria; Bacillus; Brochothrix; Clostridium; Enterococcus; Lactobacillus; Leuconostoc; and Staphylococcus. Such as Listeria monocytogenes; Bacillus coagulans spores; Bacillus licheniformis; Bacillus licheniformis spores; Bacillus subtilis spores; Brochothrix thermosphacta; Clostridium perfringens; Clostridium sporogenes spores; Enterococcus faecalis; Enterococcus gallinarum; Lactobacillus farciminis; Lactobacillus fermentum; Lactobacillus plantarum; Lactobacillus sakei; Leuconostoc mesenteroides; Listeria innocua; Staphylococcus aureus; and Staphylococcus epidermidis.

Examples of fungal contaminant microorganisms include bacteria from the genera: Aspergillus; Candida; Debaryomyces; Kluyveromyces; Penicillium; Pichia; Rhodotorula; Saccharomyces; and Zygosaccharomyces. Such as Aspergillus parasiticus; Aspergillus versicolor; Candida parapsilosis; Candida tropicalis; Citrobacter freundii; Debaryomyces hansenii; Kluyveromyces marxianus; Penicillium commune; Pichia anomala; Rhodotorula glutinis; Rhodotorula mucilaginosa; Saccharomyces cerevisiae; and Zygosaccharomyces bailii.

In one embodiment, the contaminant microorganism is selected from one or more the following genera: Salmonella and Escherichia. For example, the contaminant microorganism may be selected from one or more of the following species: Salmonella enterica or Escherichia coli.

In another embodiment, the contaminant microorganism may be selected from: Salmonella enterica subsp. enterica strains, e.g. Salmonella enterica ser. Anatum; Salmonella enterica ser. Braenderup; Salmonella enterica ser. Derby; Salmonella enterica ser. Enteritidis; Salmonella enterica ser. Hadar; Salmonella enterica ser. Infantis; Salmonella enterica ser. Kedougou; Salmonella enterica ser. Mbandaka; Salmonella enterica ser. Montevideo; Salmonella enterica ser. Neumuenster; Salmonella enterica ser. Newport; Salmonella enterica ser. Ohio; Salmonella enterica ser. Schwarzengrund; Salmonella enterica ser. Senfienberg; Salmonella enterica ser. Tennessee; Salmonella enterica ser. Thompson; and Salmonella enterica ser. Typhimurium.

Depending on the product that the anti-contaminant composition is being used with, the contaminant microorganism(s) may vary. By way of example, if the product is pet food (e.g. semi-moist pet food, e.g. kibble form or other forms of pet food or pet treats), then the contaminant microorganism may be from the genus Salmonella, e.g. from the species Salmonella enterica.

For example, if the product is pet food (e.g. kibble), then the contaminant microorganism may be Salmonella enterica ser.: Infantis or Tennessee, Salmonella enterica ser.: Senftenberg or Montevideo.

If the product is pet food, then the contaminant microorganism may be Salmonella enterica ser.: Senftenberg or Montevideo, for example.

If the product is a pet treat, then the contaminant microorganism may be Salmonella enterica ser.: Typhimurium, Newport, Anatum, Ohio, Senftenberg, Thompson, or Neumuenster, for example.

If the product is raw pet food, then the contaminant microorganism may be Salmonella enterica ser.: Hadar, Braenderup, or Schwarzengrund, for example.

If the product is frozen pet food, then the contaminant microorganism may be Salmonella enterica ser. Mbandaka, for example.

If the product is pig ear treats, then the contaminant microorganism may be Salmonella enterica ser. Infantis, for example.

If the contaminant microorganism originates from a pet food plant, then the contaminant microorganism may be Salmonella enterica ser. Derby, for example.

If the product is a foodstuff (e.g. a human foodstuff), then the contaminant microorganism(s) may vary, for example.

If the product is a human food product (e.g. a dairy product, e.g. a milk based product), then the contaminant microorganism may be selected from one or more of the following genera: Escherichia and Salmonella, for example.

In one embodiment, when the product is a foodstuff (e.g. a human foodstuff), then the contaminant microorganism may be Salmonella, for example.

In one embodiment, when the product is a foodstuff, the contaminant microorganism may be a Salmonella enterica, for example.

In one embodiment, when the product is a foodstuff, the contaminant may be selected from one or more Salmonella enterica subsp. enterica strains: Salmonella enterica ser. Anatum; Salmonella enterica ser. Braenderup; Salmonella enterica ser. Derby; Salmonella enterica ser. Enteritidis; Salmonella enterica ser. Hadar; Salmonella enterica ser. Infantis; Salmonella enterica ser. Kedougou; Salmonella enterica ser. Mbandaka; Salmonella enterica ser. Montevideo; Salmonella enterica ser. Neumuenster; Salmonella enterica ser. Newport; Salmonella enterica ser. Ohio; Salmonella enterica ser. Schwarzengrund; Salmonella enterica ser. Senftenberg; Salmonella enterica ser. Tennessee; Salmonella enterica ser. Thompson; and Salmonella enterica ser. Typhimurium, for example.

In yet another embodiment, when the product is a foodstuff (e.g. a human foodstuff), the contaminant microorganism may be Escherichia. Suitably, the contaminant microorganism may be Escherichia coli.

In another embodiment, when the product is a foodstuff (e.g. a human foodstuff), the contaminant microorganism may be one or more Escherichia coli strain selected from the group consisting of: E. coli DCS 15 (e.g. E. coli O157:H7), E. coli DCS 492, E. coli DCS 493, E. coli DCS 494, E. coli DCS 495, E. coli DCS 496, E. coli DCS 497, E. coli DCS 546, E. coli DCS 558, E. coli DCS 1336, and E. coli DCS 1396.

If the product is a dairy product (e.g. a milk based product), then the contaminant microorganism may be selected from one or more of the following genera species: Escherichia coli and Salmonella enterica, e.g. Salmonella enterica ser.: Typhimurium, Senftenberg, or Enteritidis.

### V. Additional Components

In one aspect, the compositions disclosed herein may comprise one or more additional component(s). Preferably, any additional component(s) do not materially affect the anti-contaminant properties of the composition of the present invention.

In one embodiment, the additional component(s) may be a carrier, an adjuvant, a solubilizing agent, a suspending agent, a diluent, an oxygen scavenger, an antioxidant, a food material, an anti-contaminant agent, or combinations thereof.

In another embodiment, the additional component(s) may be required for the application to which the antimicrobial is to be utilized. For example, if the anti-contaminant composition is to be utilized to on, or in, an agricultural product, the additional component(s) may be an agriculturally acceptable carrier, excipient, or diluent. Likewise, if the anti-contaminant composition is to be utilized to on, or in, a foodstuff, the additional component(s) may be an edible carrier, excipient, or diluent.

In one aspect, the one or more additional component(s) is a carrier, excipient, diluent, oxygen, scavenger, antioxidant, and/or a food material.

“Carriers” or “vehicles” mean materials suitable for compound administration and include any such material known in the art, such as, for example, any liquid, gel, solvent, liquid diluent, solubilizer, or the like, which is non-toxic and which does not interact with any components of the composition in a deleterious manner.

Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Examples of excipients include one or more of: microcrystalline cellulose and other celluloses, lactose, sodium citrate, calcium carbonate, dibasic calcium phosphate, glycine, starch, milk, sugar, and high molecular weight polyethylene glycols.

Examples of diluents include one or more of: water, ethanol, propylene glycol and glycerin, and combinations thereof.

The other components may be used simultaneously (e.g. when they are in admixture together or even when they are delivered by different routes) or sequentially (e.g. they may be delivered by different routes).

The composition or its diluent may also contain chelating agents such as EDTA, citric acid, tartaric acid, etc. Moreover, the composition or its diluent may contain active agents selected from fatty acids esters such as mono- and diglycerides, non-ionic surfactants such as polysorbates, phospholipids, etc. Emulsifiers may enhance the stability of the composition, especially after dilution.

### VI. Anti-Contaminant Agents

In one aspect, the compositions, anti-contaminant compositions, and cell-free fermentation products disclosed herein may comprise one or more additional anti-contaminant agent.

The term “additional anti-contaminant agent” refers to an anti-contaminant agent that is not produced by culturing a strain of Paenibacillus polymyxa, Paenibacillus polymyxa ABP-166, a variant of Paenibacillus polymyxa ABP-166, a derivative of Paenibacillus polymyxa ABP-166, or combinations thereof.

Such “additional anti-contaminant agents” may include: antimicrobial agents, anti-bacterial agents; anti-fungal agents; and/or anti-viral agents.

In one embodiment, the additional anti-contaminant agent is a food grade anti-contaminant.

In another embodiment, the additional anti-contaminant agent (or food grade anti-contaminant agent) is one or more of the group consisting of: food grade organic acids; a plant antimicrobial, for example, a ca tech in (e.g. from Green tea), an allylisothiocyanate (e.g. from mustard oil); a phenol (e.g. from rosemary), a plant essential oil; a bacteriocin, an anti-microbial emulsifler, fatty acid, or their esters.

A. Oxygen Scavenger

In one embodiment, the compositions, anti-contaminant compositions, or cell-free fermentation products disclosed herein may comprise an oxygen scavenger. Without wishing to be bound by theory, an oxygen scavenger may serve to preserve an anti-contaminant activity of the anti-contaminant composition or cell-free fermentation product disclosed herein. Preservation of the anti-contaminant activity may be achieved by inhibition of oxidation of components within the anti-contaminant composition or cell-free fermentation product.

Regulating the exposure of the fermentation product (or composition comprising the fermentation product) to oxygen (such as through the use of an oxygen scavenger or antioxidant) advantageously helps to maintain the anti-contaminant activity. Thus, the “shelf-life” of the product to which an anti-contaminant composition is applied may advantageously be extended. For example, by limiting the exposure of oxygen sensitive food products to oxygen in a packaging system, the quality or freshness of food may be maintained, contaminant reduced, and/or the food shelf-life extended.

In the food packaging industry, several means for regulating oxygen exposure are known, including modified atmosphere packaging (MAP) and oxygen barrier film packaging.

Regulation of oxygen exposure may be achieved by “active packaging”, whereby the package containing the food product is modified in some manner to regulate the food's exposure to oxygen. One form of active packaging uses oxygen-scavenging sachets which contain a composition which scavenges the oxygen through oxidation reactions. One type of sachet contains iron-based compositions which oxidize to their ferric states. Another type of sachet contains unsaturated fatty acid salts on a particulate adsorbent. Yet another sachet contains metal/poly amide complex.

Another type of active packaging involves incorporating an oxygen scavenger into the packaging structure itself. A more uniform scavenging effect through the package is achieved by incorporating the scavenging material in the package instead of adding a separate scavenger structure (e.g., a sachet) to the package. This may be especially important where there is restricted airflow inside the package. In addition, incorporating the oxygen scavenger into the package structure provides a means of intercepting and scavenging oxygen as it permeates the walls of the package (herein referred to as an “active oxygen barrier”), thereby maintaining the lowest possible oxygen level in the package.

Any known oxygen scavenger may be used in accordance with the present invention. A person of ordinary skill in the art can select an oxygen scavenger appropriate to the intended use of the anti-contaminant composition. For example, for food applications a person of ordinary skill in the art may use an oxygen scavenger which has GRAS approval.

Compounds which can be present or incorporated in the packaging material which scavenge oxygen include:
iron powder oxidation (such as commercially available products Ageless®, ATCO® O₂-absorber, Freshilizer®, Vitalon®, and Freshpax®);
ascorbic acid oxidation;
enzymatic oxidation (e.g. glucose oxidase and alcohol oxidase) including commercially available products such as Bioka O₂-absorber;
unsaturated fatty acids (e.g. oleic acid or linolenic acid); and
immobilized yeast on a solid material.

In one embodiment, such compounds can be used in conjunction with modified atmosphere packaging.

In one aspect at least one oxygen scavenger may be added after culturing of the one or more Bacillus subtilis strains in accordance with the present invention.

In another embodiment, at least one oxygen scavenger may be added to the cell-free fermentation product, a supernatant, a fraction, or a component thereof.

B. Antioxidant

In one embodiment, the compositions, anti-contaminant compositions, and cell-free fermentation products disclosed herein may comprise an antioxidant, and/or the containing (e.g. packaging) of the products, and/or compositions of the present invention may comprise a compound which is an antioxidant.

In one embodiment, an antioxidant may be used in the compositions and product of the present invention.

In another embodiment, an antioxidant may be used in the methods disclosed herein. For example, an antioxidant may be added prior to, during or after culturing. Without wishing to be bound by theory, an antioxidant may serve to preserve an anti-contaminant activity of the anti-contaminant composition or cell-free fermentation product of the present invention. Preservation of the anti-contaminant activity may be achieved by inhibition of oxidation of components within the anti-contaminant composition or cell-free fermentation product.

The term “antioxidant,” as used herein, refers to a molecule capable of inhibiting the oxidation of other molecules.

In one embodiment, at least one antioxidant may be added after culturing of the one or more Bacillus subtilis strains, in accordance with the present invention.

In another embodiment, at least one antioxidant may be added to the cell-free fermentation product, a supernatant, a fraction, or a component thereof.

Antioxidants are widely known and commercially available. A person of ordinary skill in the art is able to select an antioxidant appropriate for the desired end use. For example, where the anti-contaminant composition is to be used in foodstuffs, natural antioxidants such as ascorbic acid, tocopherols, butylated hydroxyanisole, and butylated hydroxytoluene may be used.

In one embodiment, a suitable antioxidant may be selected from the group consisting of: ascorbic acid, polyphenols, vitamin E, beta-carotene, rosemary extract, mannitol, and BHA.

In yet another embodiment, between about 0 ppm to about 900 ppm of an antioxidant may be added to the anti-contaminant composition of the present invention, about 0 ppm to about 100 ppm, about 100 ppm to about 200 ppm, about 200 ppm to about 300 ppm, about 300 ppm to about 400 ppm, about 400 ppm to about 500 ppm, about 500 ppm to about 600 ppm, about 600 ppm to about 700 ppm, about 700 ppm to about 800 ppm, and about 800 ppm to about 900 ppm. In other aspects more than about 900 ppm of an antioxidant may be added.

In one embodiment, between about 600 ppm to about 900 ppm of an antioxidant may be added to the anti-contaminant composition of the present invention.

In another embodiment, between about 600 ppm to about 900 ppm of ascorbic acid may be added to the anti-contaminant composition of the present invention.

### VII. Products

Products which comprise strains, compositions, anti-contaminant compositions, and cell-free fermentation products disclosed herein are provided.

Any product which is susceptible to contaminant (preferably microbial contaminant) is encompassed herein. Such products include: foodstuffs, surface coating materials, flowers, and house plants.

A. Foodstuff

A Paenibacillus polymyxa strain, strain ABP-166, compositions, anti-contaminant compositions, and cell-free fermentation products disclosed herein may be used as—or in the preparation of—a food. Herein, the term “foodstuff” is used in a broad sense—and covers food for humans as well as food for animals (i.e. a feedstuff).

In one embodiment, the term “foodstuff,” as used herein, may mean a foodstuff in a form which is ready for consumption. Alternatively or in addition, however, the term “foodstuff”, as used herein, may mean one or more food materials which are used in the preparation of a foodstuff.

The terms “foodstuff” and “food product,” as used herein, are interchangeable. The food may be in the form of a solution or as a solid—depending on the use and/or the mode of application and/or the mode of administration.

When used in the preparation of a foodstuff, compositions, anti-contaminant compositions, and cell-free fermentation products disclosed herein may be used in conjunction with one or more of: a nutritionally acceptable carrier, a nutritionally acceptable diluent, a nutritionally acceptable excipient, a nutritionally acceptable adjuvant, or a nutritionally active ingredient.

A Paenibacillus polymyxa strain, strain ABP-166, compositions, anti-contaminant compositions, and cell-free fermentation products disclosed herein may be used to reduce or prevent microbial contaminant of various foodstuffs. In one embodiment, a foodstuff or food product in accordance with the present disclosure, may be or may include: raw meat, cooked meat, raw poultry products, cooked poultry products, raw seafood products, cooked seafood products, ready-to-eat food, ready-made meals, pasta sauces, pasteurized soups, mayonnaise, salad dressings, oil-in-water emulsions, margarines, low fat spreads, water-in-oil emulsions, eggs, egg-based products, dairy products, cheese spreads, processed cheese, dairy desserts, flavored milks, cream, fermented milk products, cheese, butter, condensed milk products, ice cream mixes, soya products, pasteurized liquid egg, purees, juices, bakery products, confectionery products, fruit, fruit products, canned foods, and foods with fat-based or water-containing fillings.

In one embodiment, the foodstuff or food product is a fruit, including but not limited to apples, apricots, bananas, oranges, pears, nectarines, figs, dates, raisins, plums, peaches, apricots, blueberries, strawberries, cranberries, berries, cherries, kiwis, limes, lemons, melons, pineapples, plantains, guavas, prunes, passion fruit, tangerines, grapefruit, grapes, watermelon, cantaloupe, honeydew melons, pomegranates, and persimmons.

In another embodiment, the foodstuff or food product is a vegetable, including but not limited to artichokes, bean sprouts, beets, cardoon, chayote, endive, leeks, okra, green onions, seal lions, shallots, parsnips, sweet potatoes, yarns, asparagus, avocados, kohlrabi, rutabaga, eggplant, squash, turnips, pumpkins, tomatoes, potatoes, cucumbers, carrots, cabbage, celery, broccoli, cauliflower, radishes, peppers, spinach, mushrooms, zucchini, onions, peas, beans, and other legumes.

In yet another embodiment, the foodstuff or food product is a nut, including but not limited to almonds, cashews, peanuts, pecans, and walnuts.

In one embodiment, the foodstuff is a ready-to-eat food. The term “ready-to-eat food” as used in herein means a foodstuff that is edible without further preparation to achieve food safety. Such products include: chopped vegetables, pre-washed salads, prepared and pre-washed fruits, and processed meats.

In one embodiment, the foodstuff is a ready-made meal. The term “ready-made meal” refers to a food which has undergone one or more preparation steps prior to being sold. Ready-made meals include refrigerated and frozen ready meals that may simply be heated prior to consumption.

In one embodiment, the foodstuff may be a packaged foodstuff such as a packaged salad, ready-meal, a packaged meat product, and the like. In this aspect, the anti-contaminant composition of the present invention may be applied, in or on, the food product. In addition, or in the alternative, the anti-contaminant composition may be used in, or on, the packaging. For example, the anti-contaminant composition may be applied to the packaging.

In another embodiment, the food stuff is or includes a ready-made meal.

In one embodiment, the foodstuff may be an egg, a liquid egg, an egg-substitute, an egg-white, or an egg-based product. Egg-based products may include, but are not limited to: cake, mayonnaise, salad dressings, sauces, ice creams, and the like.

The term “constituent” refers to the use of one or more materials used to prepare the product. Thus, in the context of a foodstuff, the “constituent” will be one or more food materials used in the preparation of the foodstuff. Suitably, the anti-contaminant composition of the present invention can be used in, or on, a constituent of the foodstuff.

The term “human foodstuff,” as used herein, refers to a foodstuff which is for consumption (or primarily for consumption) by humans. In one embodiment, the term “human foodstuff”, as used herein, excludes feedstuffs for animal consumption as defined herein.

In other embodiments, the disclosure relates to a method comprising applying a Paenibacillus polymyxa strain, strain ABP-166, or a strain having all the identifying characteristics of strain ABP-166, to a food product. In still another embodiment, the invention relates to a method comprising applying a cell-free fermentation product of a Paenibacillus polymyxa strain or strain ABP-166 or a derivative, or variant from ABP-166, to a food product. A Paenibacillus polymyxa strain, strain ABP-166, compositions, anti-contaminant compositions, and cell-free fermentation products, or combinations thereof, can be applied in a single application or multiple applications.

In certain embodiments, the disclosure relates to a method for inhibiting or impeding growth of microorganisms comprising applying an effective amount of a Paenibacillus polymyxa strain, strain ABP-166, compositions, anti-contaminant compositions, cell-free fermentation products disclosed herein, or combinations thereof, to a food product. In at least some embodiments, the inhibition is by at least 2% compared to an untreated control. In certain embodiments, the inhibition is by at least 5%, 10%, 15%, 20%, 25%, 30%, 35%, 40%, 45%, 50%, 55%, 60%, 65%, 70%, 75%, 80%, 85%, 90%, or 95% as compared to an untreated control. The effective amount can be administered in one or more doses.

Pathogen-contaminated food does not necessarily show any organoleptic sign of spoilage. Bacterial food poisoning may be caused by either infection of the host by the bacterial organism or by action of the toxin produced by the bacteria either in the food or the host.

Bacterial pathogens causing food poisoning or foodborne illnesses include, but are not limited to: Salmonella, Campylobacteria, Campylobacter jejuni, E. coli, Enteropathogenic E. coli, e.g., E. coli O157, Listeria monocytogenes, Clostridium perfringens, Clostridium botulinum, Vibrio parahaemolyticus, Bacillus cereus, and Yersinia enterocolitica.

Foodstuff can be treated with strain ABP-166, compositions, anti-contaminant compositions, cell-free fermentation products disclosed herein or combinations thereof to suppress growth of any pathogenic microorganism already present and to reduce the possibility of subsequent viable infection by such microorganisms. The foodstuff can be sprayed, dipped, or brushed with a suitable dilution of strain ABP-166, compositions, anti-contaminant compositions, cell-free fermentation products disclosed herein, or combinations thereof, before packaging. If so desired, the products could subsequently be washed with water before further preparation.

B. Culinary Product

In one embodiment, the foodstuff (e.g. human foodstuff) may be or may include a culinary product. In another embodiment, the culinary product may be a sauce, salad dressing, spices, seasonings, and/or soup.

In one embodiment, the foodstuff (e.g. human foodstuff) may be or may include a sauce such as a table sauce (including sauces that are used as table sauces and sauces that are multi-purpose and can be used as table sauces), a marinade, and/or a cooking sauce (e.g. during stir-frying, steaming, etc.).

In yet another embodiment, the sauce may be or may include a fermented sauce. Various types of fermented sauces exist in different regions and different variants are included for each country. Examples include: brown sauce, chili, Worcester, plum, mint sauce for meat, tartar sauce, apple sauce for meat, horse radish, cranberry sauce for meat, oyster, hoisin, etc.

In still another embodiment, the sauce may be or may include a soy based sauce or a soy-based fermented sauce. Examples include: dark soy sauce, light soy sauce, blended soy-based sauces, e.g. —teriyaki (soy sauce blended with added sugar and mirin)—sukiyaki (with added sugar, mirin and stock)—yakitori (with added mirin, sake, sugar).

In another embodiment, the sauce may be or may include a pasta sauce. Pasta sauces include sauces either added directly to cooked pasta, heated up for a few minutes beforehand, or alternatively added to fresh ingredients, e.g. meat or vegetables, and heated up to make a sauce which will then be added to cooked pasta. Examples include: Bolognese, carbonara, mushroom, tomato, vegetable, pesto, etc.

In still another embodiment, the foodstuff (e.g. human foodstuff) is or includes a wet/cooking sauce such as Liquid (i.e. non-dehydrated) recipe cooking sauces/pastes that are added to ingredients (meat and/or vegetables) to produce a meal. This also includes recipe sauces/pastes that could be added before the cooking process (marinades) and/or during the cooking process (e.g. steaming, grilling, stir-frying, stewing, etc.).

In one embodiment, the foodstuff (e.g. human foodstuff) may be or may include dry sauces/powder mixes. Such sauces include dry sauces to which boiling water or milk is added before consumption; dry recipe powder mixes and dry powder marinades. Some dry sauces may require heating over the stove for the sauce to thicken after water/milk is added. Examples include: Hollandaise sauce, white sauce, pepper sauce, sweet and sour sauce, spaghetti bolognaise, etc.

In another embodiment, the foodstuff (e.g. human foodstuff) may be or may include a salad dressing. Suitably, the dressing may include regular salad dressings (standard ready-made) and/or dried salad dressings (i.e. powders packaged in sachets that are mixed with oil/vinegar). Examples include: oil-based products, thousand island, blue cheese, Caesar, salad cream, etc. In one embodiment, the dressing may include low fat salad dressings (examples include: oil-based products, thousand island, blue cheese, Caesar, salad cream, etc.) and vinegar-based salad dressings such as vinaigrette.

In another embodiment, the foodstuff may include: other sauces, dressings, and condiments. Examples include: (1) Non-fermented table sauces; (2) Wasabi; (3) Non-recipe purees/pastes (e.g. garlic purees/pastes); (4) Dry marinades; (5) Dry recipe powder mixes (e.g. fajita spice mix); and (6) Dehydrated recipe batter/coating (used for cooking e.g. deep frying, grilling, baking).

In one embodiment, the foodstuff (e.g. human foodstuff) may be or may include: a soup such as canned soup, ready-to-eat soup, dehydrated soup, instant soup, chilled soup, UHT soup, and frozen soup.

Canned soup include, but is not limited to all varieties of canned soup in ready-to-eat or condensed (with water to be added) form. Ready-to-eat or condensed soup in “bricks” or retort pouches are also categorized as UHT soup. Examples include: mixed vegetables, pea, leek, fish, mushrooms, tomato, chicken soup, meat soup, beef soup, chicken & mushrooms, EintSpfe, etc.

Dehydrated soup refers to a powdered soup to which water is added and then cooked for a number of minutes before consumption.

Instant soup refers to a powdered soup to which boiling water is added just before consumption.

Chilled soup refers to a soup made from fresh ingredients and stored in chilled cabinets. These products usually have a limited shelf-life.

UHT soup includes all varieties of soup in ready-to-eat or condensed (with water to be added) form sold ambient (i.e. not stored in chilled cabinets). Product types include: mixed vegetables, pea, leek, fish, mushrooms, tomato, chicken soup, meat soup, beef soup, and chicken & mushrooms.

Frozen soup includes all varieties of soup sold in frozen form. Product types include: mixed vegetables, pea, leek, fish, mushrooms, tomato, chicken soup, meat soup, beef soup, chicken & mushrooms, Eintopfe, etc.

C. Meat Based Product

A meat based foodstuff (e.g. human foodstuff), according to the present invention, is any product based on meat. The meat based foodstuff is suitable for human and/or animal consumption as a food and/or a feed.

In one embodiment, the meat based food product is a feed product for feeding animals such as a pet food product, for example.

In another embodiment, the meat based food product is a food product for humans.

A meat based food product may comprise non-meat ingredients such as water, salt, flour, milk protein, vegetable protein, starch, hydrolyzed protein, phosphate, acid, spices, coloring agents, and/or texturizing agents, for example.

A meat based food product, in accordance with the present invention, preferably comprises between 5-90% (weight/weight) meat. In some embodiments, the meat based food product may comprise at least 30% (weight/weight) meat, such as at least 50%, at least 60%, or at least 70% meat.

In some embodiments, the meat based food product is a cooked meat, such as ham, loin, picnic shoulder, bacon, and/or pork belly, for example.

The meat based food product may be one or more of the following:
(a) dry or semi-dry cured meats—such as fermented products, dry-cured and fermented with starter cultures, for example, dry sausages, salami, pepperoni, and dry ham;
(b) emulsified meat products (e.g. for cold or hot consumption), such as mortadella, frankfurter, luncheon meat, and pate;
(c) fish and seafood, such as shrimps, salmon, reformulated fish products, and frozen cold-packed fish;
(d) fresh meat muscle, such as whole injected meat muscle, for example, loin, shoulder ham, and marinated meat;
(e) ground and/or restructured fresh meat—or reformulated meat, such as upgraded cut-away meat by cold setting gel or binding, for example, raw, uncooked loin chops, steaks, roasts, fresh sausages, beefburgers, meat balls, and pelmeni;
(f) poultry products—such as chicken or turkey breasts or reformulated poultry, for example, chicken nuggets and/or chicken sausages; and
(g) retorted products—autoclaved meat products such as picnic ham, luncheon meat, and emulsified products.

In another embodiment, the meat based food product is a processed meat product, such as, for example, a sausage, bologna, meat loaf, comminuted meat product, ground meat, bacon, polony, salami, or pate.

A processed meat product may be, for example, an emulsified meat product, manufactured from a meat based emulsion, such as mortadella, bologna, pepperoni, liver sausage, chicken sausage, wiener, frankfurter, luncheon meat, or meat pate.

The meat based emulsion may be cooked, sterilized, or baked, e.g. in a baking form or after being filled into a casing of, for example, plastic, collagen, cellulose, or a natural casing. A processed meat product may also be a restructured meat product such as restructured ham, for example. A meat product of the invention may undergo processing steps such as salting, e.g. dry salting; curing, e.g. brine curing; drying; smoking; fermentation; cooking; canning; retorting; slicing; and/or shredding, for example.

In one embodiment, the meat to be contacted with the anti-contaminant compositing may be minced meat.

In another embodiment the foodstuff may be an emulsified meat product.

D. Meat

The term “meat,” as used herein, means any kind of tissue derived from any kind of animal. The term meat may be tissue comprising muscle fibers derived from an animal. The meat may be an animal muscle, for example, a whole animal muscle or pieces cut from an animal muscle. The term meat encompasses meat which is ground, minced, or cut into smaller pieces by any other appropriate method known in the art.

In another embodiment, the meat may comprise inner organs of an animal, such as heart, liver, kidney, spleen, thymus, and brain, for example.

The meat may be derived from any kind of animal, such as from cow, pig, lamb, sheep, goat, chicken, turkey, ostrich, pheasant, deer, elk, reindeer, buffalo, bison, antelope, camel, kangaroo, horse, rodent, chinchilla, any kind of fish, e.g. sprat, cod, haddock, tuna, sea eel, salmon, herring, sardine, mackerel, horse mackerel, saury, round herring, Pollack, flatfish, anchovy, pilchard, blue whiting, pacific whiting, trout, catfish, bass, capelin, marlin, red snapper, Norway pout and/or hake; and any kind of shellfish, e.g. clam, mussel, scallop, cockle, periwinkle, snail, oyster, shrimp, lobster, langoustine, crab, crayfish, cuttlefish, squid, and/or octopus.

In one embodiment, the meat is beef, pork, chicken, lamb, and/or turkey.

E. Feedstuff

In one embodiment, the “product” or the “foodstuff” may be a feedstuff.

The term “feedstuff”, as used herein, means food suitable for animal consumption, such as for cows, pigs, lamb, sheep, goats, chickens, turkeys, ostriches, pheasants, deer, elk, reindeer, buffalo, bison, antelope, camels, kangaroos, horses, fish, cats, dogs, guinea pigs, and rodents, e.g. rats, mice, gerbils, and chinchillas.

The strains, compositions, anti-contaminant compositions, or the cell-free fermentation products disclosed herein may be added to the feedstuff or a component in a manner known per se.

In one embodiment, the feed may be a fodder, or a premix thereof, a compound feed, or a premix thereof. In one embodiment, anti-contaminant composition according to the present invention, may be admixed with, and/or applied onto, a compound feed, a compound feed component or to a premix of a compound feed or to a fodder, a fodder component, or a premix of a fodder.

The term “fodder,” as used herein, means any food which is provided to an animal (rather than the animal having to forage for it themselves). Fodder encompasses plants that have been cut.

The term fodder includes: hay, straw, silage, compressed and pelleted feeds, oils and mixed rations, and also sprouted grains and legumes.

Fodder may be obtained from one or more of the plants selected from: alfalfa (lucerne), barley, birdsfoot trefoil, brassicas, Chau moellier, kale, rapeseed (canola), rutabaga (swede), turnip, clover, alsike clover, red clover, subterranean clover, white clover, grass, false oat grass, fescue, Bermuda grass, brome, heath grass, meadow grasses (from naturally mixed grassland swards, orchard grass, rye grass, Timothy-grass, corn (maize), millet, oats, sorghum, soybeans, trees (pollard tree shoots for tree-hay), wheat, and legumes.

The term “compound feed” refers to a commercial feed in the form of a meal, a pellet, nuts, cake, or a crumble. Compound feeds may be blended from various raw materials and additives. These blends are formulated according to the specific requirements of the target animal.

Compound feeds can be complete feeds that provide all the daily required nutrients, concentrates that provide a part of the ration (protein, energy) or supplements that only provide additional micronutrients, such as minerals and vitamins.

The main ingredients used in compound feed are the feed grains, which include: corn, soybeans, sorghum, oats, and barley.

In one embodiment, a premix as referred to herein, may be a composition composed of micro-ingredients such as vitamins, minerals, chemical preservatives, inhibitoiy substances, fermentation products, and other essential ingredients. Premixes are usually compositions suitable for blending into commercial rations.

The feedstuff may comprise one or more feed materials selected from the group comprising: a) cereals, such as small grains (e.g., wheat, barley, rye, oats, and combinations thereof) and/or large grains such as maize or sorghum; b) by products from cereals, such as corn gluten meal, Distillers Dried Grain Solubles (DDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, and sesame; d) oils and fats obtained from vegetable and animal sources; and e) minerals and vitamins.

In one embodiment, a feedstuff may contain at least 30%, at least 40%, at least 50%, or at least 60% by weight corn and soybean meal or corn and full fat soy, or wheat meal or sunflower meal.

In addition or in the alternative, a feedstuff may comprise at least one high fiber feed material and/or at least one by-product of the at least one high fibre feed material to provide a high fiber feedstuff. Examples of high fibre feed materials include: wheat, barley, rye, oats, by products from cereals, such as corn gluten meal, Distillers Dried Grain Solubles (DDGS), wheat bran, wheat middlings, wheat shorts, rice bran, rice hulls, oat hulls, palm kernel, and citrus pulp. Some protein sources may also be regarded as high fiber protein obtained from sources such as sunflower, lupin, fava beans, and cotton.

In another embodiment, the feed may be one or more of the following: a compound feed and premix, including pellets, nuts or (cattle) cake; a crop or crop residue: corn, soybeans, sorghum, oats, barley, corn stover, copra, straw, chaff, sugar beet waste; fish meal; freshly cut grass and other forage plants; meat and bone meal; molasses; oil cake and press cake; oligosaccharides; conserved forage plants: hay and silage; seaweed; seeds and grains, either whole or prepared by crushing, milling etc.; sprouted grains and legumes; yeast extract.

As used herein, the term “applied” refers to the indirect or direct application of the compositions, anti-contaminant compositions, or cell-free fermentation products disclosed herein to the product (e.g. the feed). Examples of the application methods which may be used, include, but are not limited to treating the product in a material comprising the anti-contaminant composition, direct application by admixing the anti-contaminant composition with the product, spraying the anti-contaminant composition onto the product surface or dipping the product into a preparation of the anti-contaminant composition or coating the product with the anti-contaminant composition.

In one embodiment the anti-contaminant composition of the present invention is preferably admixed with, or applied onto, the product (e.g. feedstuff). Alternatively, the anti-contaminant composition may be included in the emulsion or raw ingredients of a feedstuff.

F. Pet Food

Microbial contamination is an increasing concern in the pet food industry due to an increased incidence of recalls. In one embodiment, the product may preferably be a pet food. The term “pet food,” as used herein, means a food suitable for consumption by a domesticated animal such as a dog, cat, horse, pig, fish, bird, hamster, gerbil, guinea pig, rodent e.g. rat, mouse, rabbit, and chinchilla.

In one aspect, the term “pet food,” as used herein, refers to a food suitable for consumption by a domesticated dog or cat. Pet foods are subject to contaminant by microorganisms such as Salmonella, Listeria, E. coli, and Clostridium. For example, dried pet food may be particularly susceptible to microbial contaminant in the post processing phase.

The present disclosure has advantageously provided Paenibacillus polymyxa strains, strain ABP-166, compositions, anti-contaminant compositions, and cell-free fermentation products disclosed herein for use in pet food, which has one or more of the following advantages: safe, palatable, cost-effective and stable, as well as effective.

The Paenibacillus polymyxa strains, strain ABP-166, compositions, anti-contaminant compositions, and cell-free fermentation products disclosed herein may be applied on, or in, the pet food itself and/or constituent(s) (e.g. ingredients) of the pet food. For example, the anti-contaminant composition may be applied on, or in, a palatant.

Examples of typical constituents found in dog and cat food include: palatants, Whole Grain Corn, Soybean Mill Run, Chicken By-Product Meal, Powdered Cellulose, Corn Gluten Meal, Soybean Meal, Chicken Liver Flavor, Soybean Oil, Flaxseed, Caramel Color, Iodized Salt, L-Lysine, Choline Chloride, Potassium Chloride, vitamins (L-Ascorbyl-2-Polyphosphate (source of vitamin C), Vitamin E Supplement, Niacin, Thiamine Mononitrate, Vitamin A Supplement, Calcium Pantothenate, Biotin, Vitamin B12 Supplement, Pyridoxine Hydrochloride, Riboflavin, Folic Acid, Vitamin D3 Supplement), Vitamin E Supplement, minerals (e.g., Ferrous Sulfate, Zinc Oxide, Copper Sulfate, Manganous Oxide, Calcium Iodate, Sodium Selenite), Taurine, L-Carnitine, Glucosamine, Mixed Tocopherols, Beta-Carotene, and Rosemary Extract.

In one embodiment, the pet food may be a wet or dry pet food, which may be in the form of a moist pet food (e.g. comprising 18-35% moisture), semi-moist pet food (e.g. 14 to 18% moisture), dry pet food, pet food supplement, or a pet treat. Some pet food forms (e.g. moist and semi-moist pet food) are particularly susceptible to contamination due to the fact that the processing conditions for preparing the pet food are not sufficient to kill all microorganisms on, or in, the pet food.

In one embodiment, the pet food may be in kibble form. In another embodiment, the pet food may be suitable for a dog or a cat. In one embodiment, the pet food may be fish food. A fish food normally contains macro nutrients, trace elements, and vitamins necessary to keep captive fish in good health. Fish food may be in the form of a flake, pellet, or tablet. Pelleted forms, some of which sink rapidly, are often used for larger fish or bottom feeding species. Some fish foods also contain additives, such as beta carotene or sex hormones, to artificially enhance the color of ornamental fish.

In yet another embodiment, the pet food may be a bird food. Bird food includes food that is used both in birdfeeders and to feed pet birds. Typically bird food comprises a variety of seeds but may also encompass suet (beef or mutton fat).

In still another embodiment, compositions, anti-contaminant compositions, and cell-free fermentation products may be incorporated within the pet food or on the surface of the pet food, such as by spraying or precipitation thereon.

In another embodiment, compositions, anti-contaminant compositions, and cell-free fermentation products are formulated for use in pet food. In this aspect, the compositions, anti-contaminant compositions, and cell-free fermentation products may comprise additional anti-contaminant agents such as phosphoric acid, propionic acid and propionates, sulfites, benzoic acid and benzoates, nitrites, nitrates, and parabens. Alternatively, the anti-contaminant agent may not comprise any chemicals.

In one embodiment, Paenibacillus polymyxa strains, strain ABP-166, compositions, anti-contaminant compositions, and cell-free fermentation products disclosed herein may be added to a pet food or constituent thereof, such that the anti-contaminant composition is present at about 0.1% to about 10%, about 0.1 to about 5%, or about 0.1 to about 3% by weight of the pet food. In one aspect the anti-contaminant composition is present at about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, or 5.0% by weight of the pet food, where any of the stated values can form an upper or lower endpoint when appropriate.

In yet another embodiment, the pet food may be a kibble (e.g. dog kibble). An illustrative method of preparing a kibble comprises the following steps:
a. preconditioning by mixing wet and dry ingredients at elevated temperature to form a kibble dough;
b. extruding the kibble dough at a high temperature and pressure;
c. drying the extruded kibble; and
d. enrobing or coating the dried kibble with topical liquid and/or dry ingredients.

In one embodiment, Paenibacillus polymyxa strains, strain ABP-166, compositions, anti-contaminant compositions, and cell-free fermentation products disclosed herein can be applied to the kibble at any stage in the process, such as at step a and/or d.

In another embodiment, the term “pet food,” as used herein, does not encompass feed for livestock animals. The term “livestock,” as used herein, refers to any farmed animal. Preferably, livestock is one or more of ruminants such as cattle (e.g. cows or bulls (including calves)), mono-gastric animals such as poultry (including broilers, chickens and turkeys), pigs (including piglets), birds, or sheep (including lambs).

G. Food Grade Films

In some embodiments, Paenibacillus polymyxa strains, strain ABP-166, compositions, anti-contaminant compositions, and cell-free fermentation products disclosed herein can be applied to surfaces, including, but not limited to food grade films and food itself. The food grade film can be made of any flexible polymer as long as it satisfies the Food and Drug Administration (FDA), direct food contact regulations, or similar regulations issued in other countries (i.e., it is a “food grade substrate”). The food grade film can consist of one or more layers.

In some embodiments, the food grade film comprises linear low density polyethylene (LLDPE) or mixtures of low density polyethylene (LDPE) and LLDPE. In other embodiments, the food grade film can be made of modified polyolefins. High modulus materials such as polypropylene, high density polyethylene (HDPE), polyvinylidene vinyl chloride (PVDC or “Saran”), and polyvinyl chloride can comprise one of the layers of the food grade film. A high modulus material reduces the tendency for the film to tangle and tends to correlate with easy tearing of the film, making it easy to cut and dispense. Toughening materials such as low density polyethylene (LDPE), linear low density polyethylene (LLDPE), blends of LDPE and LLDPE, and ethylene vinyl acetate (EVA) can comprise another layer of the food grade film. A toughening material prevents the film from tearing or splitting when trying to handle the material and, for example, unwrap the film from a container or object. In other embodiments, a layer of EVA, ethylene acrylic acid (EAA), or ethylene methacrylic acid (EMA) also helps film stick to food or containers. In a suitable embodiment, the film substrate comprises co-extruded HDPE and LDPE, or co-extruded HDPE, LDPE, and polypropylene.

In other embodiments, additives such as antioxidants (e.g., Irgafos 168™ (a phosphite) and Irganox 1010™ (a hindered phenolic) both made by Ciba-Geigy Corporation), cling additives (e.g., polyisobutylene (PIB), ethylene vinyl acetate (EVA), amorphous polypropylene, polyterpene, sorbitan monooleate, glycerol monooleate, and microcrystalline wax), anti-block additives, pigments, and the like can also be included in the film substrate.

H. Flowers

The U.S. floral industry includes: fresh cut flowers, cut cultivated greens, potted flowering plants, foliage plants, and bedding/garden plants. A major concern in the floral industry is increasing the longevity of cut flowers.

The term cut flower refers to flowers or flower buds (often with some stem and leaf) that have been cut from the plant bearing it. In one embodiment, the cut flower is removed from the plant for indoor decorative use. In yet another embodiment, cut flowers may be used in vase displays, wreaths, and garlands.

The present disclosure has advantageously provided Paenibacillus polymyxa strains, strain ABP-166, compositions, anti-contaminant compositions, and cell-free fermentation products disclosed herein that can increase the longevity of flowers. In one embodiment, flowers include, but are not limited to: Aster, Astilbe, Black-eyed Susans, Cannas, carnations, Coneflowers, Cosmos, Crocuses, Daffodils, Dahlias, Delphiniums, Gladiolus, Hyacinths, Impatiens, Irises, Lilies, Marigolds, Morning Glories, Nasturtium, Pansies, Peonies, Petunias, Phlox, Roses, Sedum, Shasta Daisies, Sunflowers, Sweet Peas, Tulips, Veronica, Yarrow, and Zinnias.

In at least some embodiments, the Paenibacillus polymyxa strains, strain ABP-166, compositions, anti-contaminant compositions, and cell-free fermentation products disclosed herein disclosed herein can be used for inhibiting microorganism that cause spoilage of cut flowers. Without being bound to any particular theory, it is thought that the compositions, anti-contaminant compositions, and cell-free fermentation products inhibit bacterial growth that clog stems and shorten vase life of cut flowers. Use of compositions, anti-contaminant compositions, and cell-free fermentation products maintain the freshness and appearance of cut flowers, floral products, decorative foliage, and other plant cuttings. For example, the vase life of cut flowers can be extended using the compositions, anti-contaminant compositions, and cell-free fermentation products.

The Paenibacillus polymyxa strains, strain ABP-166, compositions, anti-contaminant compositions, and cell-free fermentation products disclosed herein may be applied on the stems of the cut flower. In another embodiment, compositions, anti-contaminant compositions, and cell-free fermentation products can be added to water to which the flowers will be kept.

In another embodiment, Paenibacillus polymyxa strains, strain ABP-166, compositions, anti-contaminant compositions, and cell-free fermentation products disclosed herein can be applied to the flowers prior to cutting. In yet another embodiment, Paenibacillus polymyxa strains, strain ABP-166, compositions, anti-contaminant compositions, and cell-free fermentation products disclosed herein can be a constituents) (e.g. ingredients) of a flower food or flower stabilizer.

In yet another embodiment, Paenibacillus polymyxa strains, strain ABP-166, compositions, anti-contaminant compositions, and cell-free fermentation products disclosed herein disclosed herein can be used to maintain health of a house plant. The term “house plant”, as used herein, includes: but is not limited to African violets, aloe vera, Christmas cactus, geranium, jade, jasmine, peace lily, ponytail palm, spider plants, and wandering jew.

In another embodiment, Paenibacillus polymyxa strains, strain ABP-166, compositions, anti-contaminant compositions, and cell-free fermentation products disclosed herein can be added to the soil of a house plant. In still another embodiment, Paenibacillus polymyxa strains, strain ABP-166, compositions, anti-contaminant compositions, and cell-free fermentation products disclosed herein can be applied directly or indirectly to the plant.

### VIII. Forms

The Paenibacillus polymyxa strains, strain ABP-166, compositions, anti-contaminant compositions, and cell-free fermentation products may be used in any suitable form—whether when alone or when present in a composition. The Paenibacillus polymyxa strains, strain ABP-166, compositions, anti-contaminant compositions, and cell-free fermentation products may be formulated in any suitable way to ensure that the composition comprises a cell-free fermentation product comprising active compound(s) of interest.

Paenibacillus polymyxa strains, strain ABP-166 or derivatives thereof may be in the form of a dry powder that can be sprinkled on or mixed in with a product. The composition in the form of a dry powder may include: an additive such as microcrystalline cellulose, gum tragacanth, gelatin, starch, lactose, alginic acid, Primogel, or corn starch (which can be used as a disintegrating agent).

In yet another embodiment, Paenibacillus polymyxa strains, strain ABP-166, compositions, anti-contaminant compositions, and cell-free fermentation products disclosed herein can be a spray-dried fermentate re-suspended in H₂O to a percentage selected from the following: 0.05-1, 1-3, 3-5, 5-7, 7-10, 10-15, 15-20, and greater than 20%. In another embodiment, one or more than one clarification step(s) can be performed prior to spray-drying.

In one embodiment, Paenibacillus polymyxa strains, strain ABP-166, compositions, anti-contaminant compositions, and cell-free fermentation products disclosed herein can be applied in wet or partially or completely desiccated form or in a slurry, gel, or other form.

In at least some embodiments, Paenibacillus polymyxa strains, strain ABP-166, compositions, anti-contaminant compositions, and cell-free fermentation products can be freeze-dried. In at least some embodiments, Paenibacillus polymyxa strains, strain ABP-166, compositions, anti-contaminant compositions, and cell-free fermentation products can be mixed with a carrier. The carrier includes, but is not limited to: whey, maltodextrin, sucrose, dextrose, limestone (calcium carbonate), rice hulls, yeast culture, dried starch, and sodium silico aluminate. However, it is not necessary to freeze-dry the Paenibacillus before using them. The strains can also be used with or without preservatives and in concentrated, un-concentrated, or diluted form.

The strains described herein can be added to one or more carrier. Where used, the carriers) and the strain can be added to a ribbon or paddle mixer and mixed for about 15 minutes, although the timing can be increased or decreased. The components are blended such that a uniform mixture of the culture and carriers) result. The final product is preferably a dry, flowable powder.

In one embodiment, the Paenibacillus polymyxa strains, strain ABP-166, compositions, anti-contaminant compositions, and cell-free fermentation products may be formulated as a liquid, a dry powder or a granule. The dry powder or granules may be prepared by means known to those skilled in the art, such as, in top-spray fluid bed coater, in a bottom spray Wurster or by drum granulation (e.g. High sheer granulation), extrusion, pan coating, or in a micro ingredients mixer.

In another embodiment, Paenibacillus polymyxa strains, strain ABP-166, compositions, anti-contaminant compositions, and cell-free fermentation products may be provided as a spray-dried or freeze-dried powder.

In yet another embodiment, Paenibacillus polymyxa strains, strain ABP-166, compositions, anti-contaminant compositions, and cell-free fermentation products are in a liquid formulation. Such liquid consumption may contain one or more of the following: a buffer, salt, sorbitol and/or glycerol.

In one embodiment, Paenibacillus polymyxa strains, strain ABP-166, compositions, anti-contaminant compositions, and cell-free fermentation products disclosed herein may be formulated with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate, and mixtures thereof.

Paenibacillus polymyxa strains, strain ABP-166, compositions, anti-contaminant compositions, and cell-free fermentation products disclosed herein are further described by the following paragraphs.

1. An isolated Paenibacillus polymyxa strain ABP-166, accession number B-50211.
2. An isolated strain having all of the identifying characteristics of the strain of paragraph 1.
3. An isolated strain comprising a derivative or variant of paragraph 1.
4. A composition comprising a cell-free fermentation product of any one of the strains of paragraphs 1-3.
5. A composition comprising a cell-free fermentation product of any one of the strains of paragraphs 1-3 in an effective amount to inhibit microorganisms.
6. A composition comprising a cell-free fermentation product of any one of the strains of paragraphs 1-3 in an effective amount to control plant disease.
7. A composition comprising a cell-free fermentation product of any one of the strains of paragraphs 1-3 in an effective amount to extend the longevity of a foodstuff.
8. A composition comprising a cell-free fermentation product of any one of the strains of paragraphs 1-3 in an effective amount to extend the longevity of flowers.
9. A composition comprising a cell-free fermentation product of any one of the strains of paragraphs 1-3 and a foodstuff.
10. The composition of paragraph 7 or 9, wherein the foodstuff is selected from the group consisting of: human food, pet food, plant food, and feedstuff.
11. The composition of any one of paragraphs 4-10, wherein the cell-free fermentation product is at least partially desiccated.
12. The composition of any one of paragraphs 4-10, wherein the cell-free fermentation product is fully desiccated.
13. A composition comprising the strain of any one of paragraphs 1-3 and a carrier.
14. The composition of any one of paragraphs 4-12 and a carrier.
15. A composition according to any one of paragraphs 4-14, further comprising an additional component selected from the group consisting of: carrier, adjuvant, solubilizing agent, suspending agent, diluent, oxygen scavenger, antioxidant, and a food material.
16. A composition according to any one of paragraphs 4-15, wherein the composition further comprises an oxygen scavenger and/or an antioxidant.
17. A composition according to any one of paragraphs 4-16, wherein the composition further comprises one or more additional anti-contaminant agents.
18. A composition according to any one of paragraphs 4-17, wherein the composition is effective against one or more of a Gram-negative bacterium, a Gram-positive bacterium, and a fungus.
19. A composition according to any one of paragraphs 4-18, wherein the composition is effective against a plurality of microorganisms selected from the group consisting of: a Gram-negative bacterium, a Gram-positive bacterium, and a fungus.
20. A composition according to any one of paragraphs 4-19, wherein the composition is effective against one or more Gram-negative bacteria from a genus selected from the group consisting of: Escherichia; Hafnia; Klebsiella; Pseudomonas; Salmonella; Shigella; and Yersinia.
21. A composition according to any one of paragraphs 4-20, wherein the composition is effective against one or more of: Salmonella enterica; Escherichia coli; Hafnia alvei; Klebsiella oxytoca; Pseudomonas fluorescens; Pseudomonas putida; Salmonella typhimurium; Shigella flexneri; Shigella sonnei; and Yersinia enterocolitica.
22. A composition according to any one of paragraphs 4-21, wherein the composition is effective against Salmonella.
23. A composition according to paragraphs 22, wherein the Salmonella is Salmonella enterica.
24. A composition according to paragraph 21 or paragraph 23, wherein the Salmonella enterica spp. is one or more of: Salmonella enterica ser. Anatum; Salmonella enterica ser. Braenderup; Salmonella enterica ser. Derby; Salmonella enterica ser. Enteritidis; Salmonella enterica ser. Hadar; Salmonella enterica ser. Infantis; Salmonella enterica ser. Kedougou; Salmonella enterica ser. Mbandak; Salmonella enterica ser. Montevideo; Salmonella enterica ser. Neumuenster; Salmonella enterica ser. Newport; Salmonella enterica ser. Ohio; Salmonella enterica ser. Schwarzengrund; Salmonella enterica ser. Senftenberg; Salmonella enterica ser. Tennessee; Salmonella enterica ser. Thompson; and Salmonella enterica ser. Typhimurium.
25. A composition according to any one of paragraphs 4-24, wherein the composition is effective against one or more Gram-positive bacteria from a genus selected from the group consisting of: Listeria; Bacillus; Brochothrix; Clostridium; Enterococcus; Lactobacillus; Leuconostoc; and Staphylococcus.
26. A composition according to any one of paragraphs 4-25, wherein the composition is effective against one or more of: Listeria monocytogenes; Bacillus coagulans spores; Bacillus licheniformis; Bacillus licheniformis spores; Bacillus subtilis spores; Brochothrix thermosphacta; Clostridium perfringens; Clostridium sporogenes spores; Enterococcus faecalis; Enterococcus gallinarum; Lactobacillus farciminis; Lactobacillus fermentum; Lactobacillus plantarum; Lactobacillus sakei; Leuconostoc mesenteroides; Listeria innocua; Staphylococcus aureus; and Staphylococcus epidermidis.
27. A composition according to any one of paragraphs 4-26, wherein the composition is effective against one or more fungi from a genus selected from the group consisting of: Aspergillus; Candida; Debaryomyces; Kluyveromyces; Penicillium; Pichia; Rhodotorula; Saccharomyces; and Zygosaccharomyces.
28. A composition according to any one of paragraphs 4-27, wherein the composition is effective against one or more of: Aspergillus parasiticus; Aspergillus versicolor; Candida parapsilosis; Candida tropicalis; Citrobacter freundii; Debaryomyces hansenii; Kluyveromyces marxianus; Penicillium commune; Pichia anomala; Rhodotorula glutinis; Rhodotorula mucilaginosa; Saccharomyces cerevisiae; and Zygosaccharomyces bailii.
29. The composition of paragraph 26, wherein the composition is effective against Listeria monocytogenes.
30. The composition of paragraph 26, wherein the composition is effective against Clostridium perfringens.
31. A composition according to any one of paragraphs 4-30, wherein the composition is in a solid, semi-solid, liquid, or gel forms, such as, for example, tablets, pills, capsules, powders, liquids, suspensions, dispersions, or emulsions.
32. A composition according to any one of paragraphs 4-31, wherein the composition is effective against one or more of: Bordetella spp., Enterobacter spp., Erwinia spp., E. coli O157, Hafnia spp., Klebsiella spp., Proteus spp., Pseudomonas spp., Salmonella spp., Shewanella spp., Vibrio spp., Pantoea agglomerans, Buttiauxella agrestis, Carnobacterium spp., Yersinia aldovae, Bacillus subtilis, Shewanella putrefaciens, Aeromonas salmonicida, Carnobacterium diver gens, Yersinia frederiksenii, Aeromonas veronii bv. Sobria, Enterobacter kobei, Staphylococcus pasteuri, Citrobacter freundii, Chryseobacterium scophthalmum, Brevundimonas spp., Stenotrophomonas maltophilia, Yersinia spp., Pseudoalternomonas spp., Hafnia alvei, Aeromonas spp., Serratia proteamaculans, and Yersinia enterocolitica, and gram positive: Alicyclobacillus spp., Clostridium spp., Lactococcous spp., Leoconostoc spp., Staphylococcus spp., and Listeria monocytogenes, or mixtures thereof.
33. A composition according to any one of paragraphs 4-32, wherein the composition is sealed.
34. A composition according to paragraph 33, wherein the composition is hermetically sealed.
35. A direct-fed microbial composition comprising an effective amount of a strain described in any one of paragraphs 1-3 and an animal feed material, wherein said animal feed material is selected from the group consisting of: corn, dried grain, alfalfa, corn meal, and mixtures thereof.
36. A method of growing Paenibacillus polymyxa strain ABP-166 comprising:
(a) culturing Paenibacillus polymyxa strain ABP-166 on, or in any one or more substrates; and
(b) separating the strain from the substrate.
   37. A method of forming a direct-fed microbial comprising:
(a) growing in a liquid nutrient broth, a culture including any one of the strains in Claims 1-3; and
(b) separating the strain from the liquid nutrient broth.
   38. A method of producing an anti-contaminant composition comprising:
(a) culturing Paenibacillus polymyxa strain ABP-166, on, or in any one or more substrates to produce a fermentate; and
(b) separating and/or inactivating at least some viable cells.
   39. A method according to paragraph 38, wherein bacterial spores are separated from the fermentate and/or inactivated.
   40. A method according to paragraph 38 or paragraph 39, wherein the culturing in step (a) is at a pH in the pH range of 5-9.
   41. A method according to any one of paragraphs 38-40, wherein the method comprises one or more separation and/or isolation steps to produce a supernatant of the fermentate or a fraction or component thereof, prior to step (b).
   42. A method according to any one of paragraphs 38-41, wherein the method comprises one or more separation and/or isolation steps to produce a supernatant of the fermentate or a fraction or component thereof, after step (b).
   43. A method according to any one of paragraphs 38-42, wherein the culturing is step (a) is at a temperature in the range of about 10 to about 55° C.
   44. A method according to any one of paragraphs 38-43, wherein the substrate in step (a) comprises any one of the following: a carbohydrate and/or a peptone and/or a phosphate and/or a salt and/or a buffering salt.
   45. A method according to paragraph 44, wherein the substrate comprises or is any one of the following: CASO broth or TSB.
   46. A method according to any one of paragraphs 38-45, wherein the method comprises the addition of an oxygen scavenger.
   47. A method according to any one of paragraphs 38-46, wherein the method comprises the addition of an antioxidant.
   48. A method according to paragraph 47, wherein the antioxidant is one or more of the group consisting of: ascorbic acid, polyphenols, vitamin E, beta-carotene, rosemary extract, mannitol, and BHA.
   49. A method according to any one of paragraphs 38-48, wherein the method comprises the additional step of sealing the fermentate or supernatant, fraction, or component thereof, in a container.
   50 A method according to paragraph 49, wherein the additional step of sealing is hermetically sealing.
   51. An anti-contaminant composition produced by the method of any one of paragraphs 38-50.
   52. A method of preventing and/or reducing microbial contaminant of a product comprising: contacting a constituent of the product, the product itself, and/or the packaging of the product with a strain according to any one of paragraphs 1-3.
   53. A method according to paragraph 52, wherein the product is a foodstuff selected from the group consisting of: human foodstuff, a meat product, a fruit, a vegetable, a pet food, and an animal feed.
   54. A method according to paragraph 53, wherein the foodstuff is a human foodstuff.
   55. A method according to paragraph 53, wherein the foodstuff is a pet food.
   56. A method according to paragraph 52, wherein the product is a flower.
   57. A method according to any one of paragraphs 52-56, wherein the step of contacting comprises admixing a constituent of the product with the strain.
   58. A method of preventing and/or reducing microbial contaminant of a product comprising: contacting a constituent of the product, the product itself, and/or the packaging of the product with a composition of any one of paragraphs 4-35.
   59. A method according to paragraph 58, wherein the product is a foodstuff selected from the group consisting of: a human foodstuff, a meat product, a fruit, a vegetable, a pet food, and an animal feed.
   60. A method according to paragraph 59, wherein the foodstuff is a human foodstuff.
   61. A method according to paragraph 59, wherein the foodstuff is a pet food.
   62. A method according to paragraph 58, wherein the product is a flower.
   63. A method according to any one of paragraph 58-62, wherein the step of contacting comprises admixing a constituent of the product with the composition.
   64. A method of preventing and/or reducing microbial contaminant of a product comprising: the step of contacting a constituent of the product, the product itself, and/or the packaging of the product with an anti-contaminant composition prepared in accordance with any one of paragraphs 38-51.
   65. A method according to paragraph 64, wherein the product is a foodstuff selected from the group consisting of: human foodstuff, a meat product, a fruit, a vegetable, a pet food, and an animal feed.
   66. A method according to paragraph 65, wherein the foodstuff is a human foodstuff.
   67. A method according to paragraph 65, wherein the foodstuff is a pet food.
   68. The method of paragraph 64, wherein the product is a flower.
   69. A method according to any one of paragraphs 64-68, wherein the step of contacting comprises admixing a constituent of the product with the anti-contaminant composition.
   70. A method of inhibiting microorganisms comprising: administering the isolated strain of any one of paragraphs 1-3, and/or the composition according to any one of paragraphs 4-35, and/or the anti-contaminant composition prepared in accordance with any one of paragraphs 38-51 to an environment potentially containing microorganisms.
   71. The method of paragraphs 56, 62, and 68, wherein the flowers are fresh-cut flowers.
   72. A method of inhibiting food spoilage comprising applying the isolated strain of any one of paragraphs 1-3, and/or the composition according to any one of paragraphs 4-35, and/or the anti-contaminant composition prepared in accordance with any one of paragraphs 38-51 to a food grade film.
   73. A method according to any one of paragraphs 52-72, wherein the method prevents and/or reduces microbial contaminant by one or more of a Gram-positive bacterium, a Gram-negative bacteria, or a fungus.
   74. A method according to any one of paragraphs 52-73, wherein the method prevents and/or reduces microbial contaminant by at least one Gram-positive bacterium, at least one Gram-negative bacteria, and at least one fungus.
   75. A method according to any one of paragraphs 52-74, wherein the method prevents and/or reduces microbial contaminant by one or more Gram-negative bacteria from a genus selected from the group consisting of: Escherichia; Hafnia; Klebsiella; Pseudomonas; Salmonella; Shigella; and Yersinia.
   76. A method according to any one of paragraphs 52-75, wherein the method prevents and/or reduces microbial contaminant by one or more of: Salmonella enterica; Escherichia coli; Hafnia alvei; Klebsiella oxytoca; Pseudomonas fluorescens; Pseudomonas putida; Salmonella typhimurium; Shigella flexneri; Shigella sonnei; and Yersinia enterocolitica.
   77. A method according to any one of paragraphs 52-75, wherein the method prevents and/or reduces microbial contaminant by Salmonella.
   78. A method according to paragraph 77, wherein the Salmonella is Salmonella enterica.
   79. A method according to paragraph 78, wherein the Salmonella enterica spp is one or more of the following: Salmonella enterica ser. Anatum, Salmonella enterica ser. Braenderup; Salmonella enterica ser. Derby; Salmonella enterica ser. Enteritidis; Salmonella enterica ser. Hadar; Salmonella enterica ser. Infantis; Salmonella enterica ser. Kedougou; Salmonella enterica ser. Mbandak; Salmonella enterica ser. Montevideo Salmonella enterica ser. Neumuenster, Salmonella enterica ser. Newport, Salmonella enterica ser. Ohio; Salmonella enterica ser. Schwarzengrund; Salmonella enterica ser. Senftenberg; Salmonella enterica ser. Tennessee; Salmonella enterica ser. Thompson; and Salmonella enterica ser. Typhimurium.
   80. The method according to any one of paragraph 52-79, wherein the bacteria is selected from at least one of Bordetella spp., Enterobacter spp., Erwinia spp., E. coli O157, Hafnia spp., Klebsiella spp., Proteus spp., Pseudomonas spp., Salmonella spp., Shewanella spp., Vibrio spp., Pantoea agglomerans, Buttiauxella agrestis, Carnobacterium spp., Yersinia aldovae. Bacillus subtilis, Shewanella putrefaciens, Aeromonas salmonicida, Carnobacterium divergens, Yersinia frederiksenii, Aeromonas veronii bv. Sobria, Enterobacter kobei, Staphylococcus pasteuri, Citrobacter freundii, Chryseobacterium scophthalmum, Brevundimonas spp., Stenotrophomonas maltophilia, Yersinia spp., Pseudoalternomonas spp., Hafnia alvei. Aeromonas spp., Serratia proteamaculans, and Yersinia enterocolitica, and gram positive: Alicyclobacillus spp., Clostridium spp., Lactococcous spp., Leoconostoc spp., Staphylococcus spp., and Listeria monocytogenes, or mixtures thereof.
   81. The method of any one of the preceding paragraphs, wherein the bacteria is E. coli O157.
   82. The method of any one of the preceding paragraphs, wherein the bacteria is Listeria monocytogenes.
   83. The method of any one of the preceding paragraphs, wherein the bacteria causes at least one of food spoilage, food poisoning, foodborne illness, and spoilage of cut flowers.
   84. The method of any one of the preceding paragraphs, wherein the bacteria causes spoilage of at least one of fresh meat, fruit juices, infant formula, vegetables, and mixtures thereof.
   85. The method of any one of the preceding paragraphs, wherein the bacteria causes aerobic chicken spoilage.
   86. The method of any one of the preceding paragraphs, wherein at least 1% w/v of the supernatant is used.
   87. The method of any one of the preceding paragraphs, wherein supernatant is administered to food grade film.
   88. The method of any one of the preceding paragraphs, wherein administration of the strain and/or supernatant reduces odors from the bacteria.
   89. A food grade film comprising a strain according to paragraphs 1-3.
   90. A food grade film comprising the composition according to any one of paragraphs 4-35.
   91. A food grade film comprising an anti-contaminant composition produced in accordance with any one of paragraphs 38-51.
   92. The food grade film of any one of paragraphs 89-91, wherein the film is a high barrier film.
   93. The food grade film of any one of paragraphs 89-92, wherein the film is substantially unpermeable.
   94. A feed composition comprising an effective amount of a strain described in any one of preceding paragraphs and a carrier.
   95. Use of isolated strain of Paenibacillus described herein in preparation of a medicament to prove inhibitory activity against one or more harmful microorganisms.
   96. Use of isolated strain of Paenibacillus described herein in preparation of a medicament to inhibit one or more harmful microorganisms.

### EXAMPLES

The invention will be further understood by reference to the following non-limiting examples. The following Examples are provided for illustrative purposes only. The Examples are included herein solely to aid in a more complete understanding of the presently described invention. The Examples do not limit the scope of the invention described or claimed herein in any fashion.

### Example 1

### Use of a Cell-Free Fermentation Product of ABP-166 to Inhibit E. coli O157

This experiment was aimed at investigating the potential use of a cell-free fermentation product to reduce the native microflora associated with fresh spinach, as well as determine if this treatment can reduce the prevalence of E. coli O157, an important human food pathogen that has been the cause of gastroenteritis disease associated with the consumption of contaminated spinach.

### Experimental Design

Commercially available bags of pre-washed spinach were purchased from a local grocery store and 22 g samples were aseptically transferred onto Styrofoam plates. The individual spinach leaves were arranged on the plates using a sterile stick to produce a single layer of leaves, 100 μl from an overnight culture of E. coli O157 was spotted onto the leaves in 10 spots with each spot containing 10 μl. This isolate has previously been shown to contain four virulence genes: eae, fliC stx1 and stx2. It was also positively identified as E. coli O157 using the Oxoid Agglutination Diagnostic testing kit.

The culture was allowed to dry onto the spinach surface for 1 hour at room temperature. The spinach was then aseptically transferred to sterile bags. Either 50 ml of sterile peptone or 50 ml of a crude, cell-free fermentation product of ABP-166 (produced in Tryptic-Soy Broth) was added to the appropriate bag and placed onto an orbital shaker for 15 minutes. After 15 minutes, 150 ml of sterile peptone was added to each bag and mixed thoroughly using mechanical mastication. This dilution had been shown previously to produce a cell-free fermentation product of ABP-166 with no impact on the growth of E. coli O157. Samples from these bags were serially diluted and plated onto E. coli O157 Chromogenic Agar using a spiral plater. The plates were incubated for 24 hr at 37° C. The plates were enumerated using an optical scanner.

### Results

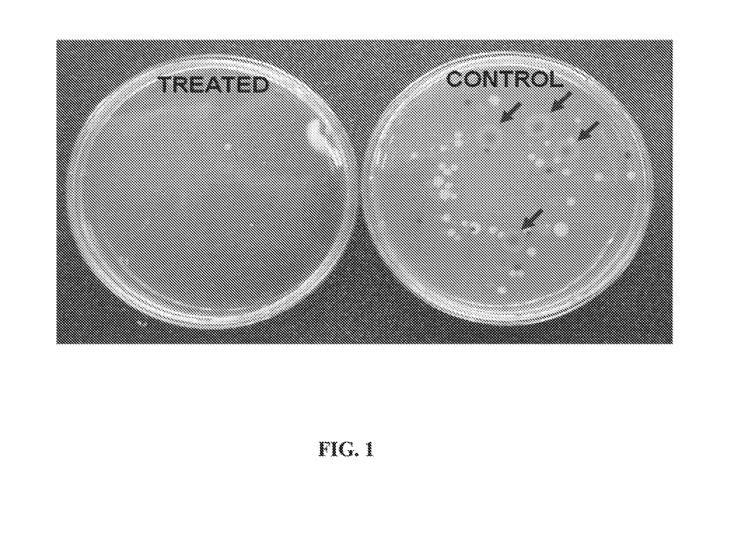

Table 2 indicates that the cell-free fermentation product of ABP-166 effectively reduced the native bacterial population by roughly 99.5% and the E. coli O157 population by approximately 95% in 15 minutes. This reduction can also be visualized in a side-by-side comparison of the 10⁻⁵ dilution plates in FIG. 1. E. coli O157 turns bright purple when grown on this media.

TABLE 2
Cell-free fermentation product of ABP-166 inhibits E. coli 0157.

| | Cell-free fermentation | | |
|---|---|---|---|
| | product of ABP-166 | Peptone | Percent Reduction |
| Native Microflora | 6.4 × 10⁴ | 2.43 × 10⁷ | 99.5% |
| E. coli O157 | 7.8 × 10⁵ | 1.87 × 10⁷ | 95% |

A cell-free fermentation product of ABP-166, even with minimal processing, can reduce the levels of bacteria that are associated with the outer cuticle surface of freshly bagged spinach. Cell-free fermentation product of ABP-166 inhibited both a human pathogen, E. coli O157 and the normal microbial-flora associated with these products with minimal physical disruption to the leaves themselves. No differences were visible between the spinach that was treated with cell-free fermentation product of ABP-166 when compared to the leaves treated with peptone.

### Example 2

### Use of Cell-Free Fermentation Product of ABP-166 to Control Alicyclobacillus Spp. Spoilage of Fruit Juices

Spoilage of fruit juices as a result of the Gram-positive, spore-forming bacteria Alicyclobacillus spp., is currently a major issue of economic importance to the fruit juice industry (see Table 3). The fruit juice industry has acknowledged Alicyclobacillus spp. as a major quality control target microorganism (Chang, S. S. and Kang, D. H., 2004). This experiment assessed efficacy of cell-free fermentation product of ABP-166 in controlling Alicyclobacillus spp. outgrowth.

TABLE 3
List of Alicyclobacillus spp. Isolates.
Alicyclobacillus spp. Isolates

| Isolate | Identity | Origin |
|---|---|---|
| 1101 | Alicyclobacillus | Nina Parkinson, National Food |
| | acidoterrestris | Processors Association, |
| | | Commercial apple juice |
| | | (ATCC 49025 |
| 1016 | Alicyclobacillus | Nina Parkinson, National Food |
| | acidoterrestris | Processors Association, |
| | | Commercial apple juice |
| 1035 | Alicyclobacillus | Nina Parkinson, National Food |
| | acidocaldorius | Processors Association |
| 14-1 | Alicyclobacillus spp. | Commercial apple juice |
| 16-1 | Alicyclobacillus spp. | Commercial apple juice |
| 18-1 | Alicyclobacillus spp. | Commercial apple juice |
| 2056 | Alicyclobacillus spp. | Commercial peach juice concentrate |
| 5301 | Alicyclobacillus spp. | Commercial apricot juice concentrate |
| 5700 | Alicyclobacillus spp. | Commercial peach juice concentrate |

### Experimental Design

Table 3 lists the isolates used to determine if cell-free fermentation product of ABP-166 could effectively inhibit Alicyclobacillus spp. growth. All isolates were obtained from the DongHyun Kang Lab at the Department of Food Science and Nutrition, Washington State University (WSU). A 10% (v/v) cell-free fermentation product of ABP-166 using TSB media was used for all of the assays. A bacteriocin spot plate assay was used to assess efficacy among the isolates for a preliminary visual representation. Briefly, the isolates (target) were seeded (1/100) into YSG agar, cell-free fermentation product of ABP-166 was serially diluted, spotted onto the target agar, and plates were incubated at 45° C. for 48 hours.

To determine specifically, the reduction capacity of cell-free fermentation product of ABP-166, Alicyclobacillus spp. were grown in YSG broth and bacterial counts were compared to Alicyclobacillus spp. grown in YSG broth with 10% cell-free fermentation product of ABP-166 after incubation at 45° C. for 48 hours. This experiment aimed at identifying cell-free fermentation product of ABP-166 activity at early stages (0, 2, 4, 6, & 24 hours after treatment with cell-free fermentation product of ABP-166).

### Results

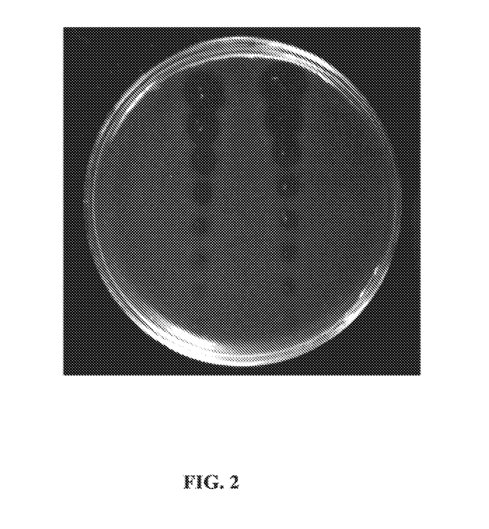

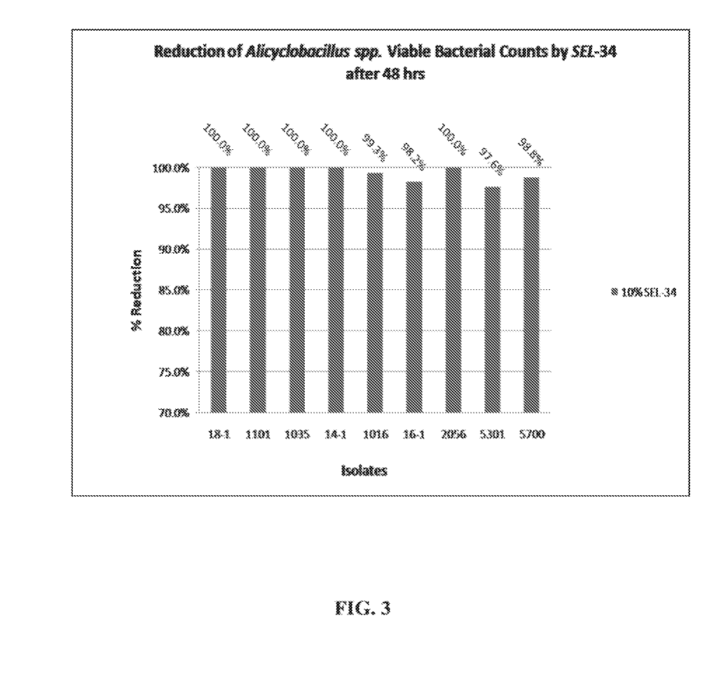

FIG. 2 shows dilutions of cell-free fermentation product of ABP-166 spotted onto a YSG agar plate containing the Alicyclobacillus spp. isolate 1101. Only seven dilutions were performed and as is shown, cell-free fermentation product of ABP-166 had activity against Alicyclobacillus spp. at a very low concentration (1:128, lowest dilution tested). Similar efficacy results were seen for the remaining isolates obtained from the Kang Lab (data not shown). Cell-free fermentation product of ABP-166 had activity against Alicyclobacillus spp.

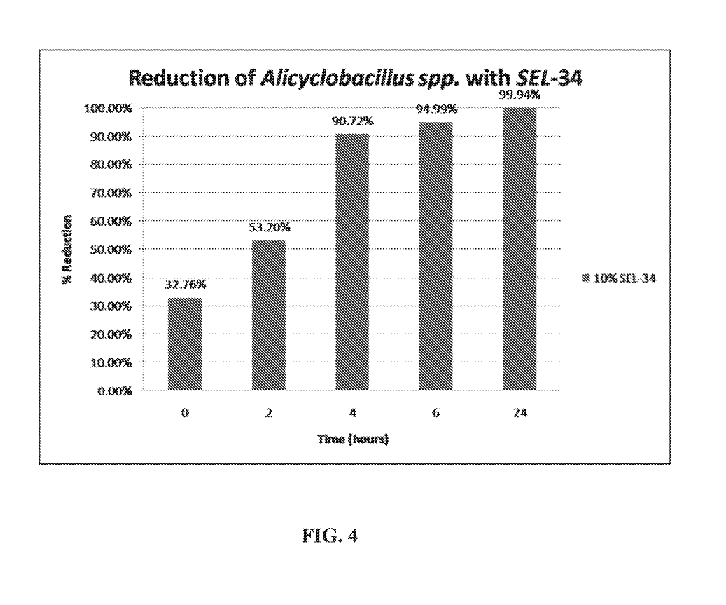

Forty-eight hour viable plate counts comparing Alicyclobacillus spp. grown in YSG broth to Alicyclobacillus spp. grown in YSG broth with 10% cell-free fermentation product of ABP-166 were determined. FIG. 4 shows the reduction of Alicyclobacillus spp. (all 9 isolates) in the presence of cell-free fermentation product of ABP-166 antimicrobial after 48 hours of incubation.

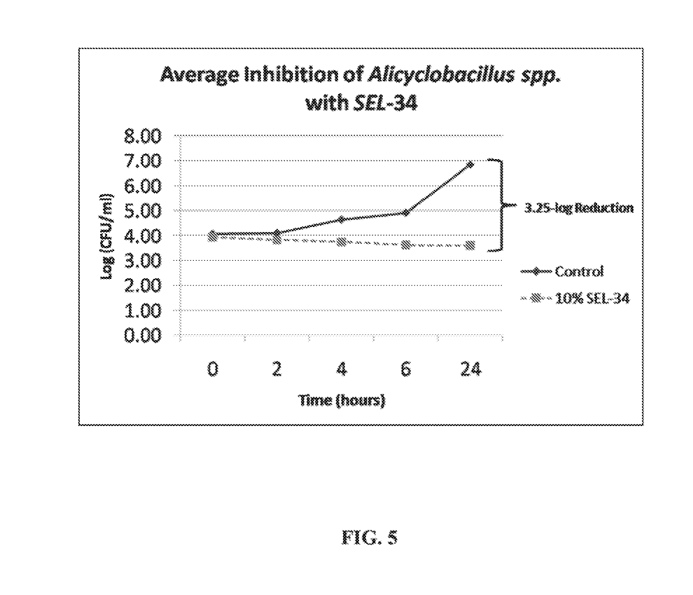

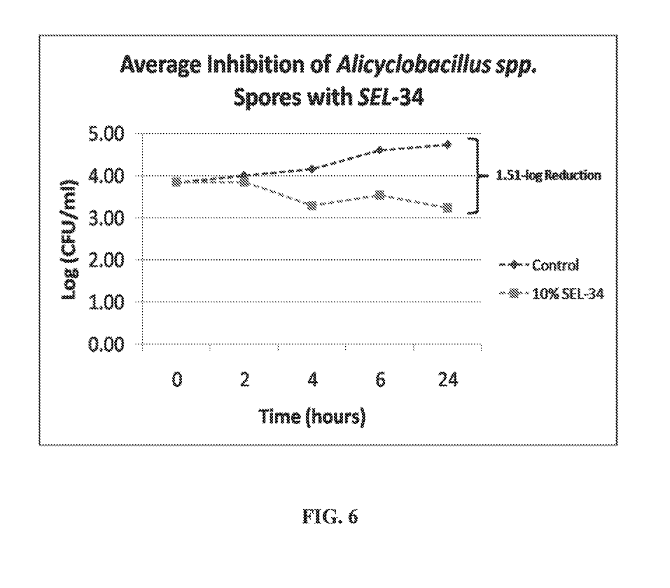

A growth inhibition assay was performed to look at cell-free fermentation product of ABP-166 activity over time (0, 2, 4, 6, and 24 hours). Vegetative and spore counts were determined from Alicyclobacillus spp. grown in YSG with 10% cell-free fermentation product of ABP-166 versus the control grown in YSG. Two isolates were chosen for this experiment, 16-1 and 1101. The data represented in FIGS. 4, 5, and 6 are the results from the averages of both isolates. FIG. 4 shows the total plate count reduction over time. Significant reductions were seen early on and by 24 hours of incubation with cell-free fermentation product of ABP-166. Alicyclobacillus spp. plate counts were reduced to 99.94%. As seen in FIG. 5 and FIG. 6, average reductions after 24 hours resulted in a 3.25 log reduction of vegetative cells and a 1.51 log reduction of spores, respectively.

As the above data indicates, cell-free fermentation product of ABP-166 is an effective antimicrobial to reduce out-growth of the fruit juice spoilage microorganism, Alicyclobacillus spp. Cell-free fermentation product of ABP-166 may be combined with the pasteurization process since the cell-free fermentation product can withstand high temperatures while retaining activity. A cell-free fermentation product of ABP-166 can serve as an important processing addition that can contribute to the control of Alicyclobacillus spp. out-growth and maintenance of fresh, unspoiled fruit juices.

### Example 3

### Use of Cell-Free Fermentation Product of ABP-166 to Control Aerobic Chicken Spoilage

Freshly packaged chicken is produced and distributed regionally since the shelf-life is short at ˜17 days (processor ˜1 day; distribution warehouse ˜9-12 days; supermarket ˜4-7 days; consumer). By gaining a better understanding of spoilage progression and of the dominant microorganisms present, applications and methodologies can be developed to improve quality and extend shelf-life. Extending the shelf-life could prove to be very beneficial for the poultry industry and ultimately to the end consumer.

### Experimental Design

The isolates chosen for further analysis, which represent the succession of microbial spoilage, consisted of isolates from the skin and skin-less samples as well as isolates from all of the sampling days. Of the 68 isolates, only 50 grew from the original stock. DNA was isolated from the 50 isolates that grew and 16S rDNA sequencing was used to determine the dominant microorganisms from the clusters. Only 37 of the 50 isolates yielded good quality sequence data. The Ribosomal Database was used to determine the identity of the microorganisms. A bacteriocin assay with cell-free fermentation product of ABP-166 was tested for activity against the 50 representative spoilage isolates.

Table 4 outlines the progression of microorganisms present over the sampling days. During the earlier days, prior to the expiration date, the dominant microorganisms were Gram-positive. The majority of microorganisms that comprise the initial flora of the carcass are generally Gram-positive (Micrococcus, Staphylococcus, and Bacillus) mesophiles, which are derived mainly from soil and fecal organisms as well as contaminants during processing (Pearson, A. M. and Dutson, T. R., 1986).

### Results

The results of the CK3 sequence data were similar to the results of the earlier trials (CK1 & CK2), in that the overall trend was for the Gram-negative microorganisms to become the more predominant microorganisms as spoilage progressed. A trend seen in the CK1 and CK2 trials, but not seen in the CK3 trial was that Serratia spp. and Pseudomonas spp. could be isolated throughout the progression of spoilage. Pseudomonas spp. were unable to be recovered from any of the current trial time points, as was seen from the sequence data. Not all of the isolates that represented major clusters could be grown or sequenced. Even though Pseudomonas spp. could not be recovered, other spoilage microorganisms were able to be identified, such as Serratia spp., Enterobacter spp., Aeromonas spp., and H. alvei.

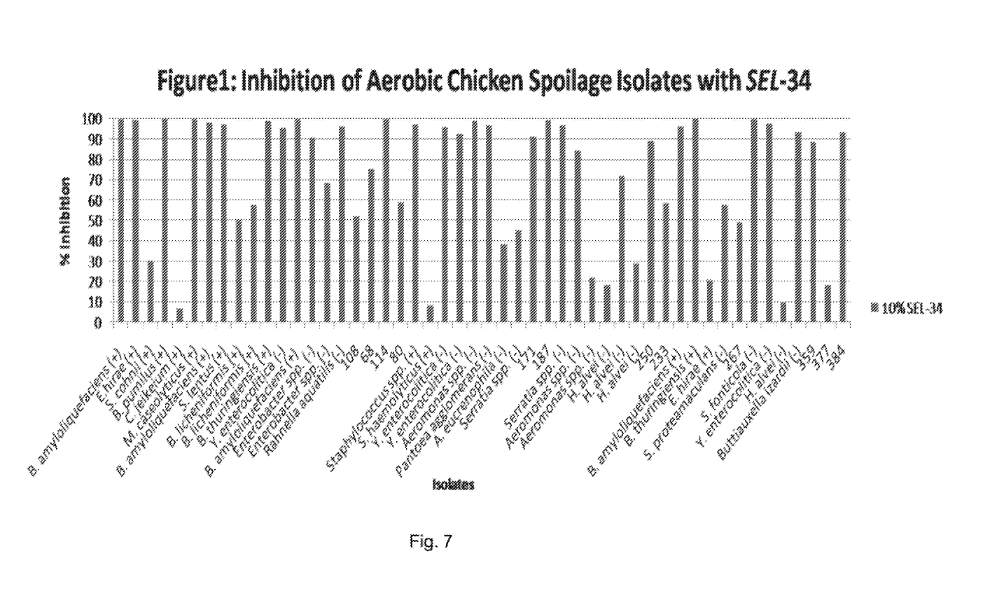

FIG. 7 is a bar graph that depicts the activity of a 10% solution of cell-free fermentation product of ABP-166 activity against the 50 isolates from the major clusters. The cell-free fermentation product of ABP-166 has a broad range of activity against both Gram-positive and Gram-negative microorganisms present in this study.

TABLE 4
Progression of microorganisms present over the sampling days.

| Sampling | | |
|---|---|---|
| Day | Cluster | Target Organism |
| 4 | 16 | B. amyloliquefaciens (+) |
| | 33 | *E. hirae (+) |
| | 36 | *S. cohnii (+) |
| | 38 | B. pumilus (+) |
| 7 | 3 | *C. jelkeium (+) |
| | 8 | M. caseolyticus (+) |
| | 16 | B. amyloliquefaciens (+) |
| | 34 | *S. lentus (+) |
| 11 | 21 | *B. licheniformis (+) |
| | 27 | *B. licheniformis (+) |
| | 45 | *B. thuringiensis (+) |
| | 29 | Y. enterocolitica (−) |
| 15 | 20 | B. amyloliquefaciens (+) |
| | 22 | Enterobacter spp. (−) |
| | 25 | Enterobacter spp. (−) |
| | 48 | *Rahnella aquatilis (−) |
| 18 | 18 | Staphylcoccus |
| | | spp. (+) |
| | 23 | S. haemolyticus (+) |
| | 29 | *Y. enterocolitica (−) |
| | 29 | Y. enterocolitica (−) |
| | 34 | *Aeromonas spp. (−) |
| | 36 | Pantoea |
| | | agglomerans (−) |
| | 43 | *A. eucrenophila (−) |
| | 54 | Serratia spp. (−) |
| 21 | 2 | *Serratia spp. (−) |
| | 32 | *Aeromonas spp. (−) |
| | 34 | *Aeromonas spp. (−) |
| | 38 | H. alvei (−) |
| | 43 | H. alvei (−) |
| | 45 | H. alvei (−) |
| 23 | 7 | *B. amyloliquefaciens (+) |
| | 37 | *B. thuringiensis (+) |
| | 46 | E. hirae (+) |
| | 4 | S. proteamaculans (−) |
| 25 | 24 | *S. fonticola (−) |
| | 29 | *Y. enterocolitica (−) |
| | 38 | *H. alvei (−) |
| | 47 | *Buttiauxellaizardii (−) |

*isolates from skin-on samples Gram Reaction (+) or (−) Day 19-20 Expiration

### Example 4

### Use of Cell-Free Fermentation Product of ABP-166 to Extend Quality of Shelf-Life of Fresh Chicken

A coating of cell-free fermentation product of ABP-166 can be used with a high-barrier film. The combination of these two technologies forms a film that provides multiple hurdles, an antimicrobial and a reduced oxygen environment, designed to prolong the outgrowth of bacterial organisms. The cell-free fermentation product of ABP-166 can be applied to the food contact surface of these films and still remains active.

Technologies with the potential to extend shelf-life by as little as 3 days would greatly impact the poultry industry. One packaging method utilizes high barrier materials, which are vacuum sealed to keep the package atmosphere low in oxygen and is optimal for shelf-life extension. However, under high barrier/low oxygen conditions, the outgrowth of hydrogen sulfide producing bacteria is prevalent, resulting in bloated packages and a characteristic “confinement odor.” Therefore, the current packaging is low barrier, allowing for the unwanted gas to diffuse out of the package, which slows down this type of spoilage. Since the current packaging allows for oxygen retention, aerobic spoilage microorganisms overwhelm the environment and spoilage occurs rapidly.

Thus, if hydrogen sulfide producing bacteria could be eliminated (reduction of “confinement odor”) while utilizing a high barrier film to reduce aerobic spoilage microorganisms, shelf-life could potentially be extended resulting in an improvement in poultry quality and consumer acceptability. A double hurdle approach will be assessed for efficacy in extending the shelf-life of fresh poultry.

### Experimental Design

This study utilized a cell-free fermentation product of ABP-166 applied as a spray in conjunction with the use of vacuum sealable high barrier pouches. Researchers have previously identified and isolated the dominant H₂S-producer associated with fresh chicken, Shewanella putrefaciens, and subsequent testing has demonstrated that these organisms are sensitive to relatively low levels of a cell-free fermentation product of ABP-166 (Data not shown).

A poultry spoilage challenge model was established for impartiality of the spoilage event and was conducted utilizing a “10⁴ CFU/ml cocktail” prepared from the dominant S. putrefaciens isolates collected in previous experiments and organized into 4 major clusters at 95% similarity using RAPD PCR technology and the bioinformatics package BioNumerics.

Fresh chicken thighs were purchased direct from the regional processor and used to conduct the experiment. The design was set up to capture sampling time points prior to and after the proposed expiration date of the chicken, which included six sample time points. Freshness was guaranteed ten days post-processing. Refer to Table 5 for the layout of sampling time points in relation to the processing and expiration date (days are referenced post-processing).

TABLE 5
Sampling time points in relation to processing and expiration dates.

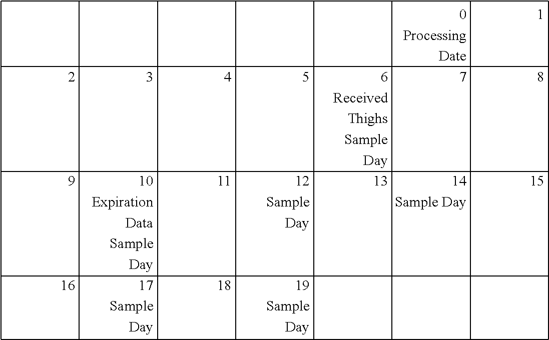

Chicken thighs were placed side-by-side on large trays and inoculated with 1 ml (10⁴ CFU/ml) of the Shewanella “cocktail” and spread onto the surface of each of the thighs. Three different spray treatments were used to assess efficacy in shelf-life extension and poultry quality. A H₂O spray treatment served as a “negative” control for the experiment and two separate spray treatments with cell-free fermentation product of ABP-166 (“test”) were used (1% and 5% (w/v) concentration). The cell-free fermentation product of ABP-166 form used for this experiment was spray-dried fermentate re-suspended in H₂O to the corresponding percentages. A clarification step conducted prior to spray-drying contributed to the elimination of some unwanted media components and nutrients. Additional clarifications procedures can be used.

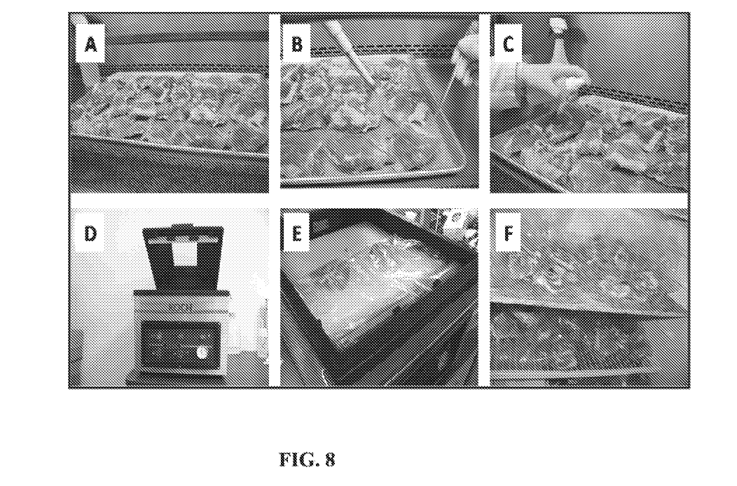

One tray per treatment of thighs was sprayed with 1 ml, using an atomizer spray bottle, onto the top surface of each thigh. Thighs were flipped and the process was repeated, yielding a total of 2 ml of treatment liquid applied to each thigh. Individual thighs were placed in a high barrier pouch and vacuum sealed using the industrial vacuum chamber. Thighs were placed at 4° C. and held at that temperature until sampling time points. FIG. 8 outlines the steps used to prepare the chicken for storage.

Microbial load, as well as consumer acceptability, which was based on odor, was tested to determine efficacy of the cell-free fermentation product of ABP-166/high barrier film approach for the reduction of poultry spoilage microorganisms as well as improved shelf-life and quality. Three different media/growth conditions were used to assess the types of microorganisms present during spoilage. The first media condition was BHI agar plates, a non-selective media, were incubated at 25° C. aerobically to determine the levels of aerobic psychrotrophic bacteria.

The second media condition was an indicator media containing BHI agar, as well as an iron and organic sulfur source, to determine hydrogen sulfide producing bacteria (black colonies, indicative of H₂S-producers due to the formation of iron sulfide) incubated at 25° C. in a candle jar to determine the levels of H₂S-producing bacteria during the experiment. Growth conditions for the indicator media were conducted at 25° C. in a candle jar since these conditions were favorable for the original isolation of Shewanella spp. isolates.

The third media condition was BHI agar plates also incubated at 25° C. in a candle jar to determine the levels of anaerobic bacteria.

An in-house sensory panel was arranged and participants were instructed to sniff poultry samples and record if there was an off-odor detected. The panel consisted of 4 participants trained in odor evaluation of chicken (trained) and participated at each sampling time point. Another group of 3 participants who had no other experience with odor evaluation of chicken (untrained) assessed for odor quality on Day 14. All participants were instructed to rate the odor quality as if they were the consumer and to keep in mind the question, “Would this chicken be acceptable for you to eat”? The odor rating was based on a 6-point scale as follows (Charles et al, 2006): 6=none detected; 5=barely detected; 4=slight odor; 3=moderate odor; 2=strong odor; and 1=extreme odor.

### Results

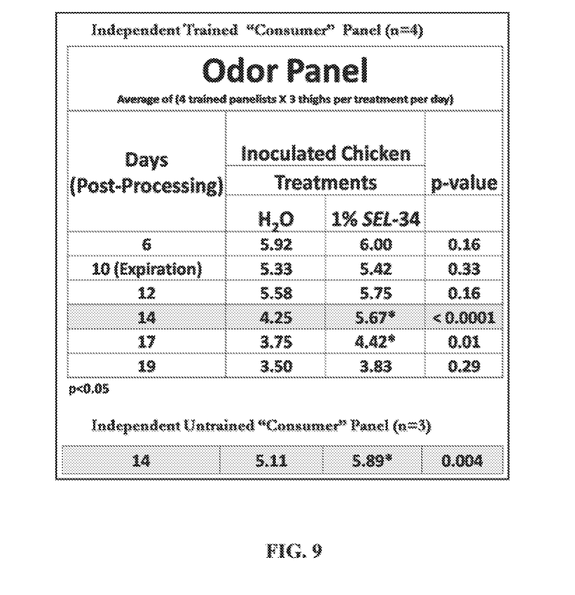

FIG. 9 represents the odor panel data observed over the 6 sampling time points for the trained “consumer” panelists as well as the day 14 data for the untrained “consumer” panelists. Overall, thighs treated with a 1% cell-free fermentation product of ABP-166 were scored with a higher odor quality rating in comparison to the H₂O treated thighs. Statistically significantly (p<0.05) higher ratings were scored for the 1% cell-free fermentation product of ABP-166 treated chicken thighs for days 4 and 7 past expiration date with scores suggesting a “barely to no detected odor” (5.67) and “slightly to barely detected odor” (4.42), respectively.

Four days past expiration date (day 14 in FIG. 9), an independent untrained group of three panelists rated both treatments. The 1% cell-free fermentation product of ABP-166 treated thighs were significantly higher with “barely to no odor detected” (5.89).

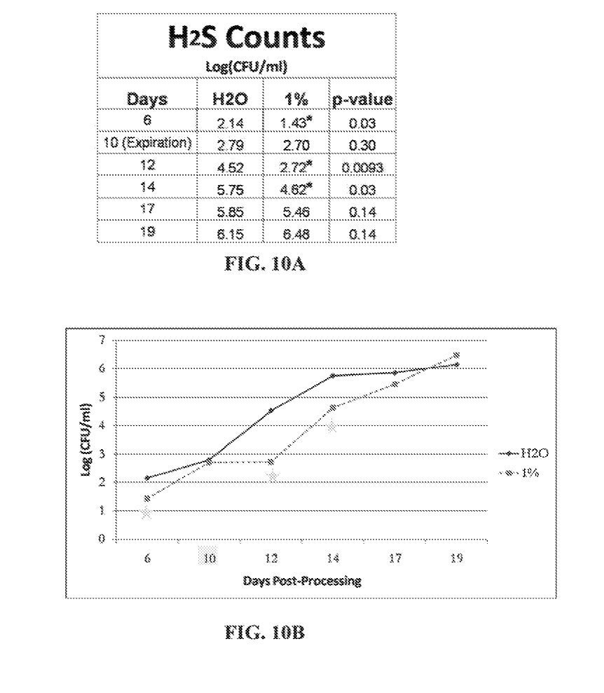

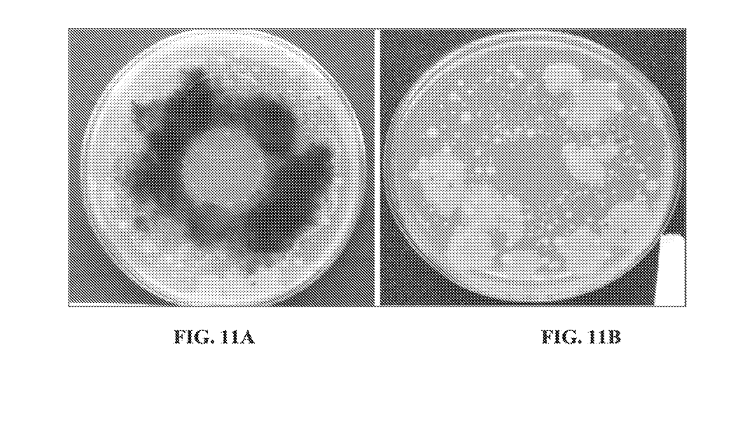

FIG. 10A is a chart and FIG. 10B is a line graph providing data from the overall reductions of H₂S-producing bacterial counts. Significant reductions were seen up to 4 days past expiration date (day 14 in FIGS. 10A and 10B). FIG. 11 is a visual representation of the reduction of H₂S-producing bacterial counts detected using H₂S media (black colonies). Both plates represent samples 2 days after expiration date. The H₂O treatment (FIG. 11A) shows a dense amount of H₂S-producing colonies, whereas the 1% cell-free fermentation product of ABP-166 treatment (FIG. 11B) shows minimal amounts of H₂S-producing colonies.

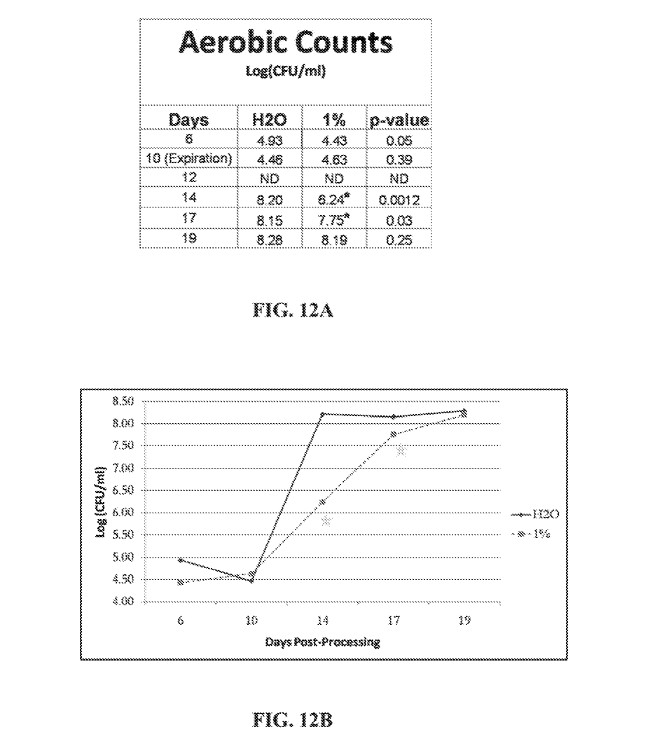

FIG. 12A is a chart and FIG. 12B is a line graph showing the reduction during progression of spoilage of total aerobic plate counts. Significant reductions were seen 7 days post-expiration date (day 17) for the 1% cell-free fermentation product of ABP-166 treated thighs.

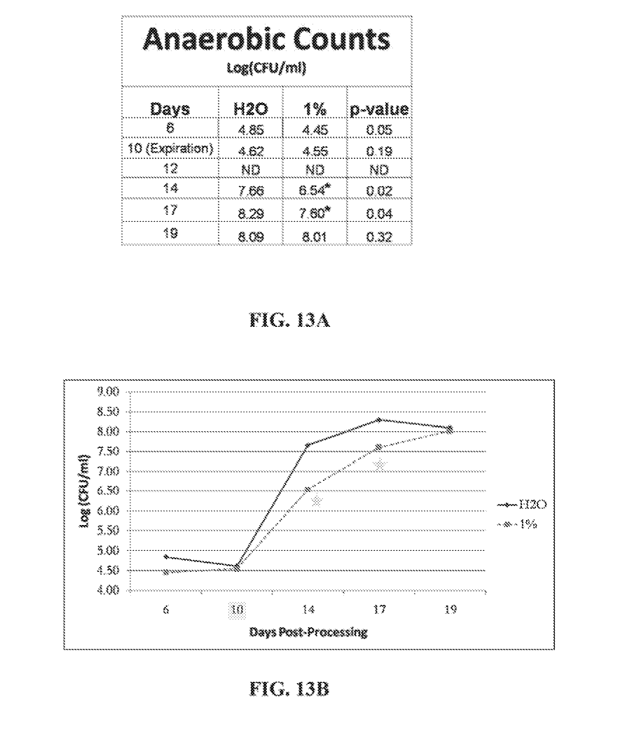

FIG. 13A is a chart and FIG. 13B is a line graph showing the reduction of total anaerobic bacteria. Total anaerobic plate counts were significantly reduced 4 and 7 days post-expiration date (days 14 and 17) for the 1% cell-free fermentation product of ABP-166 treatment in comparison to the H₂O treatment (FIG. 13A and FIG. 13B). Overall, reductions were significant for total bacterial counts (H₂S, aerobic and anaerobic), as well as significantly more favorable odor ratings for the 1% cell-free fermentation product of ABP-166 treatment.

The above data suggest that a cell-free fermentation product of ABP-166, combined with a high barrier packaging material, may reduce spoilage and extend the shelf-life of freshly packaged chicken thighs. Odor panel data was strongly correlated to plate count data, thus giving confidence for the reliability of a subjective data set. Information from the odor panel in conjunction with the plate count data demonstrated a significant improvement in quality as far out as seven days past expiration date for the 1% cell-free fermentation product of ABP-166 treated thighs in comparison to the H₂O treated thighs.

Data from the plate counts demonstrated that the spoilage event still occurs, but the rate of spoilage is delayed. The combination of data suggests that this technology can extend shelf-life and odor quality, rather than mask the spoilage event presenting a seemingly quality product to the consumer. Cell-free fermentation product of ABP-166/High Barrier Film Technology has proven to be an effective method for reducing “confinement odor” and overall bacteria counts, while maintaining a positive “consumer” acceptance of the final product.

### Example 5

### Use of a Cell-Free Fermentation Product of ABP-166 to Extend the Shelf-Life of Vacuum-Sealed Chicken

Extending the shelf-life by vacuum-sealing fresh chicken could prove to be very beneficial for the poultry industry and ultimately to the end consumer. By gaining a better understanding of the spoilage progression and of the dominant microorganisms present, Agtech can explore applications to improve quality and extend shelf-life.

### Experimental Design

Chicken legs, direct from the processor, were placed in high barrier/low oxygen packaging, vacuum-sealed, and stored at 4° C. Samples were taken according to days post-processing 1, 4, 7, 9, 11, 14, 17, and 21 and serially diluted and plated onto media to detect H₂S producing colonies, aerobic colonies at 25° C., and anaerobic colonies at 25° C. in a candle jar. Isolates were collected that had different colony morphologies within and across sampling days. Two plates (1 aerobic isolates and 1 anaerobic/H₂S isolates) were collected and stored. Genomic DNA was isolated from the collection and 16S sequencing was performed to determine the identities (Data not shown). Of the 192 isolates identified, it seems that Carnobacterium spp. represented the most dominant microorganisms of the vacuum-sealed fresh packaged chicken microbiota (15% of collected isolates). Tables 6 and 7 show the identities of the dominant microorganisms.

TABLE 6
Aerobic isolate identities and cell-free fermentation product
of ABP-166 inhibition data.

| Isolate # | Identity RDB | % Inhibition |
|---|---|---|
| A1-0-1 | Escherichia coli | 99.14 |
| A1-0-3 | Staphylococcus spp. | 93.71 |
| A4-0-6 | Pseudomonas spp. | 38.99 |
| A7-0-14 | Pantoea agglomerans | 94.76 |
| A7-0-15 | Buttiauxella agrestis | 99.16 |
| A9-0-28 | Carnobacterium spp. | 95.81 |
| A9-0-29 | Shewanella spp. | 99.82 |
| A11-4-36 | Yersinia aldovae | 99.68 |
| A11-2-41 | Bacillus subtilis | 67.68 |
| A11-2-43 | Shewanella putrefaciens | 95.77 |
| A14-4-53 | Aeromonas salmonicida | 98.89 |
| A14-4-55 | Yersinia enterocolitica | 97.72 |
| A14-2-61 | Pseudomonas spp. | No |
| | | Inhibition |
| A17-4-67 | Serratia spp. | No |
| | | Inhibition |
| A17-4-70 | Enterobacter spp. | 99.22 |
| A17-4-79 | Carnobocterium divergens | 98.10 |
| A21-6-90 | Yersinia frederiksenii | 98.03 |
| A21-6-91 | Aeromonas veronii bv. Sobria | 98.97 |

TABLE 7
Anaerobic and H₂S-producing isolate identities and cell-free
fermentation product of ABP-166 inhibition data.

| Isolate # | Identity RDB | % Inhibition |
|---|---|---|
| An7-0-7 | Pantoea agglomerans | 99.31 |
| An1-0-9 | Enterobacter kobei | 90.09 |
| An1-0-11 | Streptococcus dysgalactiae | No |
| | | Inhibition |
| An1-0-15 | Staphylococcus pasteuri | 98.66 |
| An1-0-17 | Citrobacter freundii | 73.20 |
| An1-0-18 | Chryseobacterium scophthalmum | No |
| | | Inhibition |
| An4-0-31 | Brevundimonas spp. | No |
| | | Inhibition |
| An4-0-34 | Pseudomonas spp. | 99.42 |
| An9-2-37 | Carnobacterium spp. | 97.17 |
| An9-0-41 | Stenotrophomonas maltophila | 99.40 |
| An9-0-43 | Yersinia spp. | 97.35 |
| An9-0-45 | Pseudoalteromonas spp. | 99.52 |
| H11-2-50 | Shewanella putrefaciens | 91.82 |
| H11-2-53 | Shewanella putrefaciens | 99.76 |
| H11-2-56 | Yersinia spp. | 98.63 |
| An14-4-62 | Hafnia alvel | 92.80 |
| An14-2-67 | Carnobacterium divergens | 92.10 |
| H17-6-73 | Aeromonas spp. | 99.47 |
| An17-6-74 | Yersinia frederiksenii | 97.92 |
| An17-6-79 | Serratia proteamaculans | No |
| | | Inhibition |
| An21-6-84 | Buttiauxella agrestis | 99.60 |

A broth assay was performed to determine the reduction in bacterial growth of the isolates as a result of treatment with cell-free fermentation product of ABP-166. The isolates were grown in BHI and optical density (OD₅₉₅) was compared to isolates grown in BHI with 1% cell-free fermentation product of ABP-166 after incubation at 25° C. for 24 hours (candle jar or aerobically according to original isolation conditions). A 1% (w/v) cell-free fermentation product of ABP-166 was prepared using optimized industrial media and was used for the broth assays. The dominant microorganisms from each of the aerobic and anaerobic condition were tested with cell-free fermentation product of ABP-166 to determine efficacy in inhibiting these spoilage microorganisms.

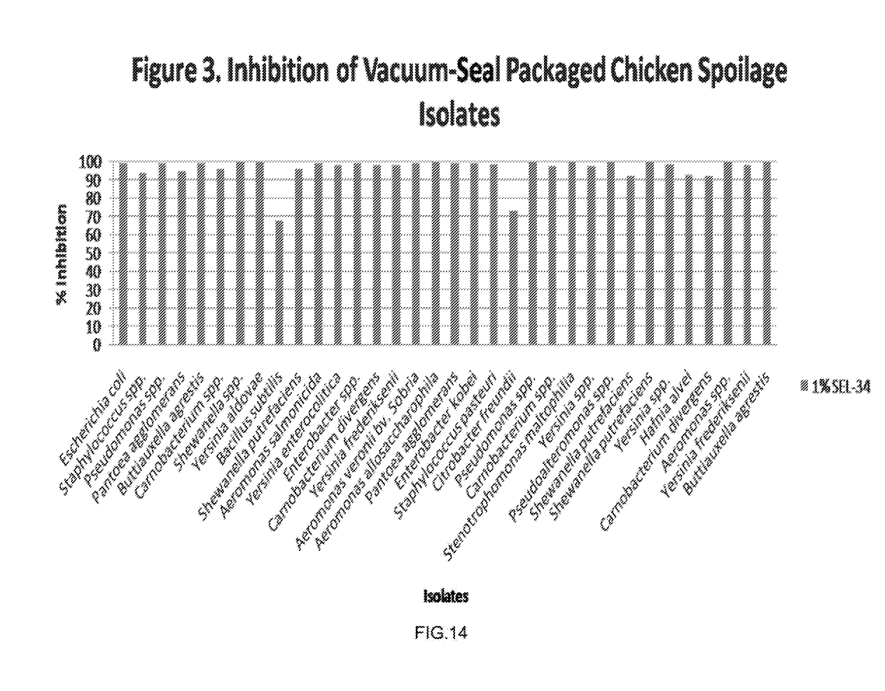

FIG. 14 represents the reduction of each isolate as a result of the addition of cell-free fermentation product of ABP-166 (Refer to Tables 6 and 7 for inhibition percentages). Overall, the average inhibition of the isolates that were inhibited by cell-free fermentation product of ABP-166 was 96.08%. Chryseobacterium scopthalmum, Streptococcus dysgalactiae, Pseudomonas spp., as well as Serratia spp. were not inhibitied by cell-free fermentation product of ABP-166. It is possible that experimental error caused the lack of inhibition or that inhibition occurred but could not be measured by the assay used. However, the data indicate that cell-free fermentation product of ABP-166 has good efficacy over a broad-range of spoilage microorganisms.

### Example 6

### Use of a Cell-Free Fermentation Product of ABP-166 to Control H₂S Producing Bacteria on Fresh Chicken

Conventionally wrapped fresh chicken is currently wrapped with plastic that has some permeability to allow for gas exchange. Fresh chicken cannot be anaerobically packed since some spoilage microorganisms produce gases (e.g. H₂S) that bloat the packaging and produce foul odors.

Anaerobically packaged chicken would reduce aerobic spoilage microbes, which would help to extend shelf-life, but the reduction of facultative and anaerobic microbes that are culprits of the production of gas still needs to be addressed. In this experiment, the dominant H₂S producers are identified and the antimicrobial activity of cell-free fermentation product of ABP-166 was tested. Thus, applications disclosed herein combining antimicrobial activity and anaerobic packaging of chicken can be assessed for efficacy in extending shelf-life.

### Experimental Design:

The anaerobic chicken survey of spoilage was conducted using fresh whole chicken carcasses from Gold N Plump. The chicken carcasses were cut in half and individual halves were packaged for each sampling time point. The chicken was stored in anaerobic boxes at 4° C. until time of sampling. The chicken was received about day 4 post-processing. Samples were taken at days 4, 6, 10, 13, 16, 18, 20, 22, and 24 (this is the expiration date). For each sampling day, 22 grams of meat and skin were combined separately with 198 ml 0.1% peptone, stomached, serially diluted, and plated onto H₂S agar.

Plates were overlaid with more H₂S agar and placed in anaerobic boxes at 37° C. Plates were assessed for black colonies (H₂S producing bacteria) and they were stored for later analysis. H₂S producing colonies were not observed until the samples from day 20 (expiration date) were plated. Anaerobically packaged chicken samples had only a slight odor at day 4 past the expiration date.

There were a total of 23 black colonies (H₂S producing bacteria) that could be isolated. 16S rDNA sequence data was used in conjunction with the Ribosomal Database to identify the H₂S producers. All isolates were identified as Shewanella putrefaciens (Gram-negative).

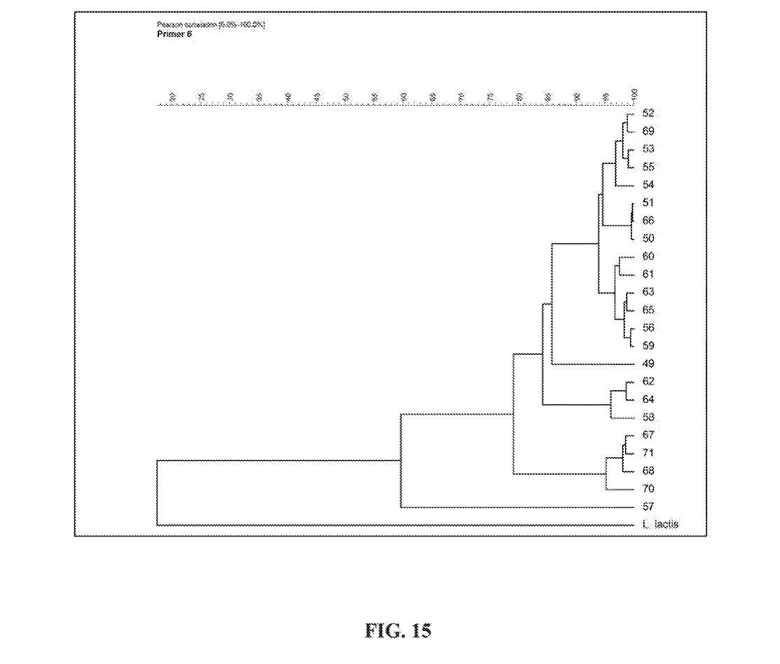

A RAPD PCR using a primer (5-CCCGTCAGCA-3′) was conducted to determine the diversity among the S. putrefaciens isolates. FIG. 15 is a dendogram that was constructed using the Pearson Correlation with 80% cut-off relatedness. Of the 23 isolates, 18 were with 80% relatedness, 4 were 79.2% relatedness, and 1 was at 59.7% relatedness (FIG. 15). Lactococcus lactis (Gram-positive) was used for a basis of comparison.

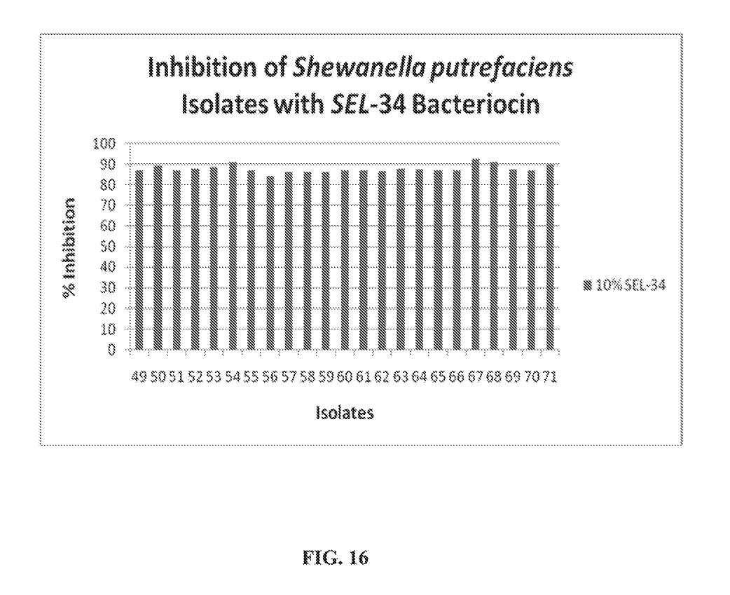

FIG. 16 is a graph that depicts the activity of 10% cell-free fermentation product of ABP-166 against the 23 S. putrefaciens isolates. The cell-free fermentation product of ABP-166 had activity against all of the isolates tested.

### Example 7

### Use of a Cell-Free Fermentation Product of ABP-166 on Food Grade Films to Inhibit Growth of Human Pathogens

A cell-free fermentation product of ABP-166 retains activity even when completely desiccated. This characteristic opens a myriad of application avenues, the most intriguing of which is its use in, or on, food grade films. Here, the use of a cell-free fermentation product of ABP-166 with food grade films was investigated.

### Experimental Design/Results

Food grade packaging films were procured from a local vendor. These films were of varying consistencies and used in many industry applications. To prepare these films for testing, a 2.2 cm² glass cover slip was attached to the non-food contact surface of these films with double sided tape. This design allowed the films to be easily manipulated. After assembly, the films were placed film side up in sterile petri dishes and placed under a UV light for 20 minutes to reduce the level of contaminating bacteria. Once finished, 1 ml of a crude version of a cell-free fermentation product of ABP-166, produced in Tryptic-Soy broth and not processed post-fermentation, was applied to the surface of each film and allowed to dry in a 30° C. incubator for 20-24 hours or until the surface appeared completely dry.

The dried films were then inverted onto the surface of nutrient agar plates that contained a suspension from an overnight culture of either E. coli O157, Salmonella, or Listeria monocytogenes. The films were gently pushed onto the agars surface using a pair of sterile forceps so that the films made consistent contact with the plate. An untreated film also was applied to these plates. These served as negative controls. The plates were placed into a 37° C. incubator for 24 hours.

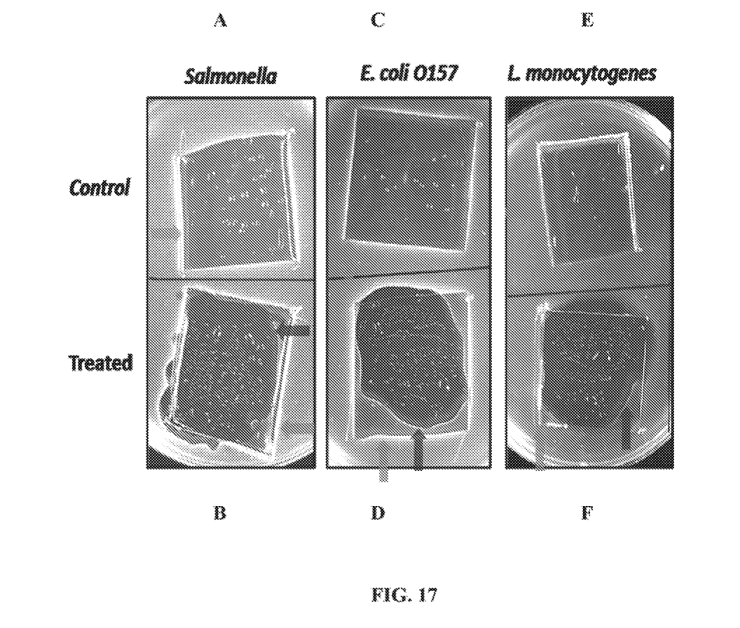

FIG. 17 shows the digital images of three human food pathogens, Salmonella (17A, and 17B), E. coli O157 (17C and 17D), and L. monocytogenes (17E and 17F) when treated with either a control film, top (FIGS. 17A, C and E), or one of the films that had been treated with cell-free fermentation product of ABP-166, bottom (FIGS. 17B, D, and F). The light colored arrows, seen in all six panels, are pointing to the outline that is left from the film being in contact with the agars surface. In the lower panels, the dark colored arrows are pointing to the irregular outline that can be seen where the application of cell-free fermentation product of ABP-166 inhibited the growth of the bacteria in the plate in contrast to the surrounding area where the bacteria was able to grow. All three pathogens (FIGS. 17B, D and F) were equally inhibited by the application of cell-free fermentation product of ABP-166.

The following test was conducted to determine if the application of cell-free fermentation product of ABP-166 to food grade films may work to reduce the levels of pathogens on typical food surfaces. Results from the previous experiment demonstrated that all four of the film types that were tested responded in a similar manner so only one film type was tested in this experiment.

To prepare the films, four 2.2 cm² glass slides were arranged in a larger square to produce a 4.4 cm² square and attached it to the non-food surface of the experimental film. Once assembled, the films were placed film side up in sterile petri dishes and placed under a UV light for 20 minutes to reduce the level of contaminating bacteria. Once finished, 3 ml of a crude version of cell-free fermentation product of ABP-166, produced in Tryptic-Soy broth and not processed post-fermentation, was applied to the surface of each film and allowed to dry in a 30° C. incubator for 20-24 hours or until the surface appeared completely dry.

Typical bologna lunch meat was bought at a local grocery store and cut into 4.4 cm² pieces and placed into sterile petri plates. The top surfaces of these pieces were inoculated with 100 μl from an overnight culture of Listeria monocytogenes and spread to cover with a sterile glass tool. The inoculated pieces of meat were then placed at 4° C. for 30 minutes to allow the bacteria to adhere to the meats surface. The plates were removed and the slices were either covered with a film that contain cell-free fermentation product of ABP-166 or an identical film that was not treated with cell-free fermentation product of ABP-166 and returned to the refrigerator. At 24 and 72 hours one treated and one untreated film/meat sample was removed from the refrigerator and the film was separated from the meat using sterile forceps. Both the meat and the film were placed into the same sterile bag and 100 ml of sterile peptone was added to the bags and they were mechanically masticated. The resulting material was serially diluted and plated.

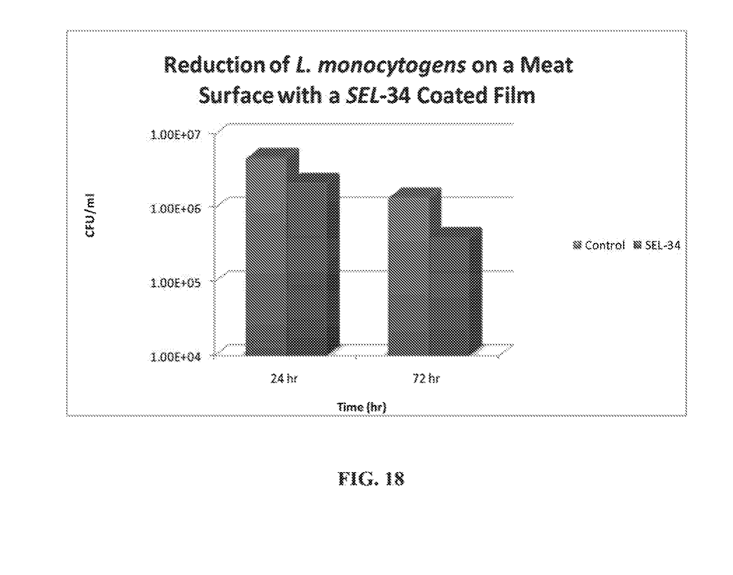

FIG. 18 shows that at 24 hours the cell-free fermentation product of ABP-166 treated sample was able to reduce the level of L. monocytogenes by ˜55% when compared to the control. Further, this reduction increased to over 70% by 72 hours when compared to the control.

The above data demonstrate that cell-free fermentation product of ABP-166 can be completely dehydrated and still remain active. The ability of cell-free fermentation product of ABP-166 to be dehydrated dramatically increases the number of applications for a cell-free fermentation product of ABP-166. This work also demonstrates that cell-free fermentation product of ABP-166 can be applied directly to the surface of food grade films through dehydration. The ability of a cell-free fermentation product of ABP-166 to be applied to various food systems through the use of an active packaging film is a key characteristic that will allow the use of cell-free fermentation product of ABP-166 in numerous methods and applications.

### Example 8

### Use of Paenibacillus polymyxa Strain ABP-166 to Inhibit Naturally Occurring Microflora Associated with Fresh-Cut Flowers

The vase-life of fresh-cut flowers is directly related to several factors. Two of the most important factors are the nutrients that are available to the plant for vascular uptake, often in the form of a plant food additive, as well as the degree of bacterial outgrowth that occurs in the vase environment. Plant food additives often consist of greater than 80% sucrose included to act as a nutrient source for the plant and to improve the vase-life of the cut flowers. However, the inclusion of sucrose also serves as a nutrient source for the bacteria that are found not only in the vase water but that are also associated with the plant material itself. If the outgrowth of these organisms is not controlled, they will form biofilms within the plants vascular structure, effectively stopping the flow of water, and thus nutrients, through the plant. This decrease in nutrients results in the premature death of the plant and shortened vase-life.

To counter this biofouling phenomenon, current plant foods include acidifying agents that help to lower the pH of the vase water, which will eliminate some bacteria while increasing the vascularization of the plants and chemical biocides like 8-hydroxyquinolone. These chemical based biocide can be harmful if administered incorrectly and do not reflect the consumer perception of fresh-flowers as “all-natural” or “environmental friendly.” In this experiment, bacteria that can be found in close association with fresh-cut flowers was collected and identified and their susceptibility to cell-free fermentation product of ABP-166 was determined.

### Experimental Design and Results

### Isolation and Identification of Dominant Flower Associated Isolates

A bunch of gladiolas and a bunch of carnations, as well as two bouquets of assorted flowers were purchased from a local grocery store. Approximately 6 inches of the stem material was removed from the end opposite the flower head, spilt longitudinally to open, and 22 g of this material was transferred to a sterile bag and 198 ml of sterile diluent was added. The samples were then masticated, serially diluted and plated onto Brain-Heart Infusion Agar plates. The plates were placed into a 32° C. incubator and allowed to grow for 2 days. The plates were removed and 50 isolates were aseptically transferred into fresh BHI broth in a 96-well plate. These isolates came from the highest dilution plates and thus represent the most prevalent bacteria in the sample that were capable of being cultured.

DNA was isolated from these samples and subjected to RAPD PCR and analyzed using a bioinformatics software package to orientate these isolated based on their genetic relatedness. DNA from representative isolates, from the most common genetic groups, were subjected to a 16s rDNA PCR and subsequent sequencing. The nucleotide sequences were then compared to a reference nucleotide library to try and categories the isolates by genus and species. A list of these key organisms as can be found in Table 8.

TABLE 8
The dominant flower-associated isolates, their suggested genus and
species and the flowers samples from which the isolates originated.

| Isolate Number | Genus | Species | Flower Type |
|---|---|---|---|
| 11 | Leuconostoc | garlicum | Gladiola |
| 49 | Lactococcous | lactis | Assorted Bouquet 1 |
| 53 | undetermined | undetermined | Assorted Bouquet 1 |
| 63 | Erwinia | crysanthemi | Assorted Bouquet 1 |
| 96 | Leuconosolc | lactis | Carnation |
| 105 | Erwinia | herbicola | Carnation |
| 106 | Leuconosloc | garlicum | Carnation |
| 107 | Bacillus | amyloliquifaciens | Carnation |
| 140 | Erwinia | herbicola | Assorted Bouquet 2 |
| 143 | Erwinia | herbicola | Assorted Bouquet 2 |
| 160 | Erwinia | herbicola | Assorted Bouquet 2 |

Assessing Microbial Sensitivity to cell-free fermentation product of ABP-166 and Two Commercial Plant Foods.

An inhibition assay was developed to assess how the addition of cell-free fermentation product of ABP-166 to simulated plant food would work in reducing the level of bacterial growth for these key isolates. This test also included the use of two common plant food products, each of which contains a chemical biocide. The conventional products were re-suspended in 475 ml sterile tap water as per labeled usage rate. The pH of these solutions was tested and determined to be 3.88 and 5.18, respectively. For controls, the pH of 2 sterile water samples was adjusted to either 3.88 or 5.18 and 0.8% sucrose was added to each sample. The addition of sucrose was between the estimated final concentrations of sucrose commonly used in these products.

For the cell-free fermentation product of ABP-166 treatment, a cell-free semi-optimized version was used, which had not been further purified. For a control, diluted growth media that contained no cell-free fermentation product of ABP-166 was used. This design resulted in 6 total treatments.

To run the inhibition assay, 1 of the 11 bacterial isolates was added to a sample of each of the 6 treatments (this was done in duplicate) and the samples were allowed to incubate at 37° C. for 24 hours. Included in these tests were samples of each treatment that contained no bacteria and served as negative controls. After the incubation, the optical density was determined for all of the samples and the background level of optical density was removed using the values from the samples that contained only growth media and no bacteria. The normalized optical density for the treated samples were then compared to the appropriate and normalized optical density in the control sample and an inhibition percentage was determined. This number reflects the percent inhibition of the treatment sample compared to the control.

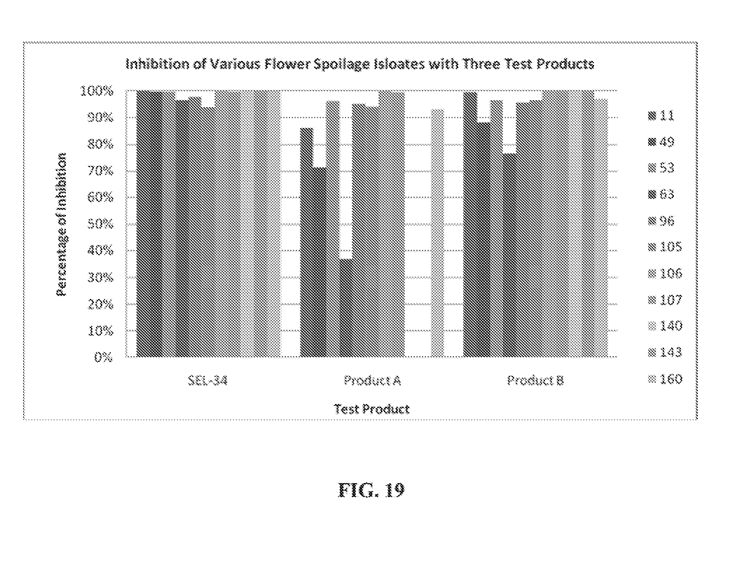

FIG. 19 shows the inhibition results for the 11 isolates when tested against full-strength cell-free fermentation product of ABP-166 and the two commercial products. This graph shows relatively high levels of inhibition for both cell-free fermentation product of ABP-166 and Product B, while Product A appeared to have limited inhibition against isolates 63, 140, and 143. This effect may be attributed to very poor growth in the control samples, which gives a false result in this type of test. The pH for Product A was only 3.88, which is very acidic, and this most likely resulted in the poor growth in the control sample. All of those isolates were Erwinia species.

This data indicated that cell-free fermentation product of ABP-166 was highly effective against all of the isolates that were tested in this study. It also suggests that when compared to the results from simulated plant food conditions, cell-free fermentation product of ABP-166 can effectively reduce the levels of bacteria at similar levels as two commercial products that contain both acidifying agents as well as chemical biocides.

### Example 9

### Use of Cell-Free Fermentation Product of ABP-166 to Control Salmonella enterica Contaminated Pet Kibble

Recent Salmonella outbreaks and pet food recalls have led to a more critical review of microbiological control programs in pet food facilities, which has brought to the forefront the need for effective control solutions (Buchanan et al.). Salmonella can enter pet food facilities via raw materials (plant and animal origin), and although extrusion often kills these heat-sensitive bacteria, common post-extrusion procedures leave potential for re-contamination (Behravesh et al. and Finn et al.). Once Salmonella enters a production environment, the identification and subsequent removal can prove challenging (Finn et al.). A safe and effective control solution can be useful as a hurdle technology and provide an additional step in controlling Salmonella enterica contamination of pet kibble (pet food).

### Experimental Design

This study utilized a cell-free fermentation product of ABP-166 that was spray dried to a free-flowing powder with a mannitol carrier. The objective was to test the efficacy of ABP-166 cell free powder to effectively inhibit differing doses of Salmonella (2×10², 10³, 10⁴, 10⁵, 10⁶ CFU/g of kibble) applied to kibble with the use of the BAX® Diagnostics equipment for detection (presence/absence) following sample enrichment. Direct plating techniques were also used at a high challenge dose (2×10⁶ CFU/g of kibble).

In brief, “naked” kibble was coated in a step-wise manner with 8% fat (v/w), 1% dry palatant (w/w), and then 1.5% (w/w) ABP-166 cell free powder. The only antimicrobial present was the ABP-166 material. Control kibble was coated in the same manner, but with 1.5% (w/w) mannitol carrier instead. Treatment and control kibble was measured to 22 g and placed into individual bags (triplicate samples) and inoculated with Salmonella enterica Newport at the appropriate doses (˜2×10², 10³, 10⁴, 10⁵, 10⁶ CFU/g of kibble).

Kibble was held at 25° C. for 24 hours and 1 week. At a given time point, all of the control and treatment samples were diluted (1:10) with sterile peptone and masticated.

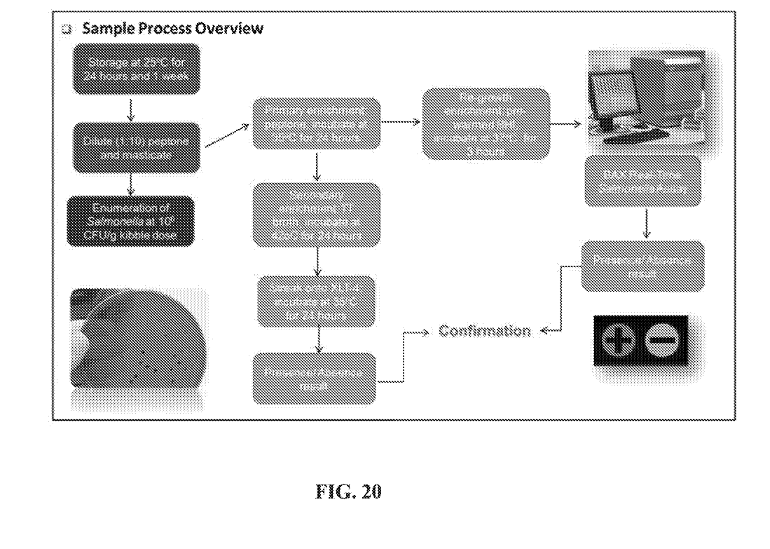

FIG. 20 provides an overview of the Salmonella detection process. The 2×10⁶ CFU/g inoculated samples were plated onto non-selective media to obtain direct plate counts.

All samples were analyzed by BAX® System diagnostics determination (presence/absence). A primary enrichment was conducted by incubating the diluted samples at 35° C. for 24 hours. As per manufacturer's instructions, a sample of the primary enrichment was transferred to pre-warmed BHI and incubated at 37° C. for 3 hours. This re-growth enrichment process is suggested for pet food samples. The BAX® System Real-Time PCR Assay for Salmonella protocol was followed and a presence or absence of Salmonella was noted.

Classical enrichment plating techniques were used to confirm the results obtained from the BAX® System. Samples were transferred from the primary enrichment to a secondary enrichment, TT broth (Hajna), and incubated at 42° C. for 24 hours. Samples from the secondary enrichment were plated onto XLT-4 plates and incubated at 35° C. for 24 hours. Plates were observed for the presence or absence of Salmonella colonies and were used as confirmation for the BAX® System result.

### Results:

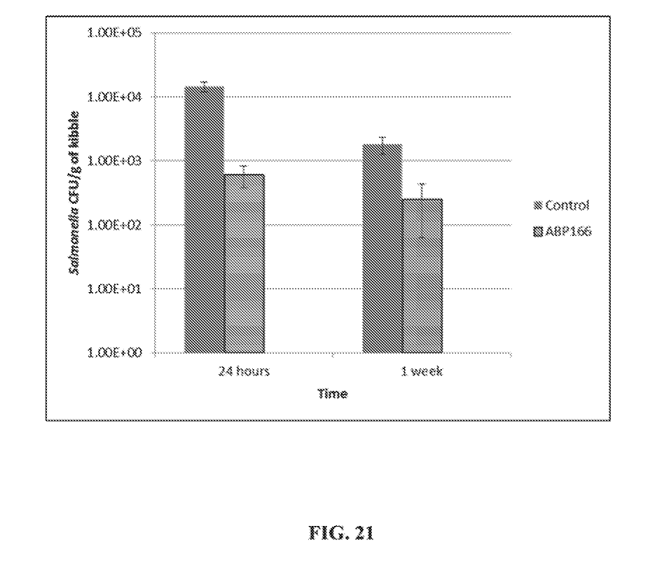

FIG. 21 and Tables 9 and 10 are the results of the direct plating methods for the high dose challenged kibble (2×10⁶ CFU/g of kibble). The ABP166 treated samples had lower amounts of Salmonella as compared to the control at 24 hours (Table 9) and 1 week (Table 10). There was a 1.38 Log reduction with the use of ABP166 in comparison to the control after 24 hours (Table 9) and a 0.87 Log reduction after 1 week (Table 10).

TABLE 9
Direct Plate Counts: The average Log (CFU/g of kibble after 24 hours)
value and the Log reduction of ABP166 versus the control.

| Sample | Average log (CFU/g) (24 hrs) | Log reduction vs control |
|---|---|---|
| Control | 4.16 | |
| ABP166 | 2.78 | 1.38 |
| Neg Control | 0.00 | |

TABLE 10
Direct Plate Counts: The average Log (CFU/g of kibble after 1 week)
value and the Log reduction of ABP166 versus the control.

| Sample | Average log (CFU/g) (1 week) | Log reduction vs control |
|---|---|---|
| Control | 3.26 | |
| ABP166 | 2.39 | 0.87 |
| Neg Control | 0.00 | |

Table 11 shows the results of the one week BAX® System Real-Time PCR Assay for the detection of Salmonella as well as the results of the classical enrichment plating techniques onto XLT-4. Both results are represented as a presence (+) or an absence (−) of Salmonella detection/recovery. ABP166 treatment of kibble challenged with 2×10² CFU/g Salmonella could not be detected by the BAX® System nor recovered on XLT-4 after 1 week, whereas Salmonella could be detected/recovered from the control sample.

TABLE 12
BAX ® System Results: The presence (+) or absence (−)
of Salmonella as was determined after 1 week by BAX ® System
detection/classical enrichment plating techniques (BAX
result/enrichment plating result).

| 1 Week | Replicate Samples | | |
|---|---|---|---|
| Treatment | 1 | 2 | 3 |
| 10² Control | +/+ | +/+ | +/+ |
| 10² ABP166 | −/− | −/− | −/− |
| 10³ Control | +/+ | +/+ | +/+ |
| 10³ ABP166 | +/+ | +/+ | +/+ |
| 10⁴ Control | +/+ | +/+ | +/+ |
| 10⁴ ABP166 | +/+ | +/+ | +/+ |
| 10⁵ Control | +/+ | +/+ | +/+ |
| 10⁵ ABP166 | +/+ | +/+ | +/+ |
| 10⁶ Control | +/+ | +/+ | +/+ |
| 10⁶ ABP166 | +/+ | +/+ | +/+ |
| Negative Control | −/− | −/− | −/− |

Overall, kibble coated with ABP166 was able to reduce the level of Salmonella at high challenge doses and eliminate the presence at low challenge doses.

It is understood that the various preferred embodiments are shown and described above to illustrate different possible features of the invention and the varying ways in which these features may be combined. Apart from combining the different features of the above embodiments in varying ways, other modifications are also considered to be within the scope of the invention. The invention is not intended to be limited to the preferred embodiments described above, but rather is intended to be limited only by the claims set out below. Thus, the invention encompasses all alternate embodiments that fall literally or equivalently within the scope of these claims. The disclosures of patents, references, and publications cited in the application are incorporated by reference in their entirety herein.

### BIBLIOGRAPHY

Buchanan, R. L., Baker, R. C., Charlton, A. J., Riviere, J. E., and Standaert. 2000. Pet food safety: a shared concern. British Journal of Nutrition. 106(S1): S78-S84.
Behravesh C B, Ferraro A, Deasy M III, Dato, V., Moll, M., Sandt, C., Rea, N. K., Rickert, R., Marrott, C., Warren, K., Urdaneta, V., Salehi, E., Villamil, E., Ayers, R., Hoekstra, R. M., Austin, J. L., Ostroff, S., Williams, I. T. 2010. Human Salmonella Infections Linked to Contaminated Dry Dog and Cat Food, 2006-2008. Pediatrics. 126(3):477-483.
Chang, S. S. and Kang, D. H. (2004) Alicyclobacillus spp. in the Fruit Juice Industry: History, Characteristics, and Current Isolation/Detection Procedures. Critical Reviews in Microbiology, 30: 55-74.
Charles, N., Williams, S. K., and Rodrick, G. E. (2006) Effects of Packaging Systems on the Natural Microflora and Acceptability of Chicken Breast Meat. Poultry Science 85:1798-1801.
Finn, S., Condell, O., McClure, P., Arnézquita A., Fanning, S. 2013. Mechanisms of survival, responses, and sources of Salmonella in low-moisture environments. Frontiers in Microbiology. 4(331) doi: 10.3389/fmicb.201300331.
Pearson, A. M. and Dutson, T. R. (1986) The Control of Microbial Spoilage in Fresh Meats. Advances in Meat Research: Volume 2 Meat and Poultry Microbiology: 58-59
Splittstoesser, D. F., Worobo, R. W., and Churry, J. J. (1998) Food Safety and You: Alicylcobacillus: An Emerging Problem for New York's Processors of Fruit Juices. Venture, Vol. 1 No. 3.

What is claimed is:

1. An isolated Paenibacillus polymyxa strain ABP-166, accession number B-50211.

2. An isolated strain having all of the identifying characteristics of the strain of claim 1.

3. A composition comprising a cell-free fermentation product of the strain of claim 1 in an effective amount to achieve a desired effect, wherein the desired effect is selected from the group consisting of: inhibit growth of microorganism, reduce the incidence or extent of plant disease, extend the longevity of a foodstuff, and extend the longevity of flowers.

4. The composition of claim 3 further comprising a foodstuff.

5. The composition of claim 4, wherein the foodstuff is selected from the group consisting of: human food, pet food, plant food, and feedstuff.

6. The composition of claim 2, further comprising an animal feed material, wherein said animal feed material is selected from the group consisting of: corn, dried grain, alfalfa, corn meal, and mixtures thereof.

7. A method of forming a direct-fed microbial comprising:
(a) growing in a liquid nutrient broth Paenibacillus polymyxa strain ABP-166, accession number B-50211; and
(b) separating the strain from the liquid nutrient broth.

8. A method of producing an anti-contaminant composition comprising:
(a) culturing Paenibacillus polymyxa strain ABP-166, on, or in any one or more substrates to produce a fermentate; and
(b) separating and/or inactivating at least some viable cells.

9. The method of claim 8, wherein bacterial spores are separated from the fermentate and/or inactivated.

10. The method of claim 8, wherein the method comprises one or more separation and/or isolation steps to produce a supernatant of the fermentate or a fraction or component thereof, prior to step (b).

11. An anti-contaminant composition produced by the method of any one of claims 8-10.

12. A method of preventing and/or reducing microbial contaminant of a product comprising: contacting a constituent of the product, the product itself, and/or packaging of the product with a composition comprising Paenibacillus polymyxa strain ABP-166, accession number B-50211.

13. A method according to claim 12, wherein the product is a foodstuff selected from the group consisting of: human foodstuff, a meat product, a fruit, a vegetable, a pet food, and an animal feed.

14. The method of claim 12, wherein the product is a flower or a fresh-cut flower.

15. A method of inhibiting growth of a microorganism comprising: applying a composition comprising Paenibacillus polymyxa strain ABP-166, accession number B-50211 to an environment potentially containing microorganisms in an effective amount to inhibit growth of a microorganism.

16. A method of inhibiting food spoilage comprising applying a composition comprising Paenibacillus polymyxa strain ABP-166, accession number B-50211 to a food grade film in an effective amount to inhibit food spoilage.

17. A method of extending the longevity or lifespan of a product comprising: contacting a constituent of the product, the product itself, and/or the packaging of the product with composition comprising Paenibacillus polymyxa strain ABP-166, accession number B-50211 in an effective amount to extend the longevity or lifespan of the product.

18. A product comprising a food grade film and Paenibacillus polymyxa strain ABP-166, accession number B-50211.

19. The product of claim 18, wherein the film is a high barrier film.

20. The product of claim 18, wherein the film is substantially unpermeable.
